(19) United States
(12) Patent Application Publication
(10) Pub. No.: US 2018/0179524 A1
(43) Pub. Date: Jun. 28, 2018

(54) COMPOSITIONS AND METHODS FOR INHIBITING CELL PROLIFERATION

(71) Applicant: President and Fellows of Harvard College, Cambridge, MA (US)

(72) Inventors: Norbert Perrimon, Arlington, MA (US); Benjamin Housden, Brighton, MA (US); Alexander Valvezan, Medford, MA (US); Brendan Manning, Needham, MA (US)

(21) Appl. No.: 15/555,331

(22) PCT Filed: Mar. 4, 2016
(86) PCT No.: PCT/US16/20906

§ 371 Date: Sep. 1, 2017

Related U.S. Application Data
(60) Provisional application No. 62/128,330, filed on Mar. 4, 2015.

(51) Int. Cl. C12N 15/113; G01N 33/574; C12Q 1/68

(52) U.S. Cl. CPC C12N 15/113; G01N 33/57492; C12Q 1/6886; C12Q 2600/156; C12N 2310/14; G01N 2333/912; C12N 15/1137

(57) ABSTRACT

The present disclosure provides, among other things, compounds that inhibit the expression or activity of gene products having a synthetic lethal interaction with loss of TSC1 and/or TSC2. Also provided are applications, such as therapeutic and diagnostic methods, in which the compounds are useful. For example, the compounds described herein can be used in methods for treating a proliferative disorder (e.g., a cancer) or an inflammatory disorder.

### RELATED APPLICATION

This application claims the benefit of priority to U.S. Provisional Patent Application Ser. No. 62/128,330, filed Mar. 4, 2015. This application is hereby incorporated herein by reference in its entirety.

### GOVERNMENT SUPPORT

This invention was made in part with government support under Grant No. W81XWH-12-1-0179 from the U.S. Department of Defense. The government may have certain rights in the invention.

### BACKGROUND

The tuberous sclerosis complex (TSC) protein complex is a point of convergence of multiple upstream signaling pathways that is vital for the control of growth and proliferation in response to extracellular signals. Genetic disruption of the TSC protein complex, through mutations in TSC1 or TSC2, gives rise to the TSC and lymphangioleiomyomatosis (LAM) diseases, which are systemic disorders associated with the development of widespread neoplastic lesions (Crino et al. (2006) N Engl J Med 355:1345). Current therapeutic strategies targeting the TSC complex and the surrounding network include the Tor inhibitor rapamycin and its derivatives. However, such treatments are limited to cytostatic effects and tumors rapidly regrow following cessation of treatment, underscoring the pressing need to identify new therapeutic targets for the treatment of TSC and other proliferative disorders. Bissler et al. (2008) N Engl J Med 358:140; McCormack et al. (2011) N Engl J Med 364:1595; Krueger et al. (2010) N Engl J Med 363:1801; Kaelin (2012) Science 337:421; and Mohr et al. (2014) Nat Rev Mol Cell Biol 15:591.

### SUMMARY

The disclosure is based, at least in part, on the discovery of several synthetic lethal interactions with tumor sclerosis complex (TSC) tumor suppressors TSC1 and TSC2. That is, TSC1 and TSC2 mutant cell lines were combined with RNAi screens against all kinases and phosphatases, to thereby identify genes whose loss of function or inhibition, in conjunction with the functional loss of TSC1 and TSC2, resulted in cytotoxicity. For example, knockdown of mRNA-cap/RNGTT, Pitslre/CDK11, or CycT/CCNT1 reduced the viability of Drosophila TSC1 or TSC2 mutant cells, but did not reduce the viability of wild-type cells. These observations indicate that inhibition of one or more of the identified genes, or constituents of the pathways (e.g., mRNA capping process) associated with these genes, is useful for the treatment of disorders associated with mutations in TSC1 or TSC2. Furthermore, as noted above, mutations in TSC1 and TSC2 are associated with upregulated mTOR activity; thus, inhibiting one or more of the subject genes is also useful for treating disorders associated with upregulated mTOR1 activity or expression.

Accordingly, in one aspect, the disclosure features a method for inhibiting the growth of a proliferating cell. The method comprises contacting a proliferating cell with a compound that inhibits IMPDH (inosine-5′-monophosphate dehydrogenase 1 or 2 (IMPDH1 or IMPDH2)), RNGTT (RNA guanylyltransferase and 5′-phosphatase), RNMT (RNA (guanine-7-) methyltransferase), Cdk11 (cyclin dependent kinase 11), Cdk9, CCNT1 (Cyclin T1), CCND3 (Cyclin D3), Cyclin L1, or Cyclin L2 in an amount effective to inhibit the growth of the cell.

In another aspect, the disclosure features a method for inhibiting cell proliferation. The method comprises contacting a proliferating cell with a compound that inhibits IMPDH (IMPDH1 or IMPDH2), RNGTT, RNMT, Cdk11, Cdk9, CCNT1, CCND3, Cyclin L1, or Cyclin L2 in an amount effective to inhibit the proliferation of the cell.

In another aspect, the disclosure features a method for reducing cell viability (e.g., inducing apoptosis). The method comprises contacting a cell (e.g., a proliferating cell) with a compound that inhibits IMPDH (IMPDH1 or IMPDH2), RNGTT, RNMT, Cdk11, Cdk9, CCNT1, CCND3, Cyclin L1, or Cyclin L2 in an amount effective to reduce the viability of the cell (e.g., inducing apoptosis).

In another aspect, the disclosure features a method for reducing cell mobility or motility (e.g., inhibiting metastasis of a cell). The method comprises contacting a cell (e.g., a proliferating cell) with a compound that inhibits IMPDH (IMPDH1 or IMPDH2), RNGTT, RNMT, Cdk11, Cdk9, CCNT1, CCND3, Cyclin L1, or Cyclin L2 in an amount effective to reduce the motility or mobility of the cell.

In another aspect, the disclosure features a method for inhibiting the growth of a proliferating cell. The method comprises contacting a proliferating cell with a compound that inhibits mRNA capping in an amount effective to inhibit the growth of the cell.

In another aspect, the disclosure features a method for inhibiting cell proliferation. The method comprises contacting a proliferating cell with a compound that inhibits mRNA capping in an amount effective to inhibit the proliferation of the cell.

In another aspect, the disclosure features a method for reducing cell viability (or, e.g., inducing apoptosis). The method comprises contacting a cell (e.g., a proliferating cell) with a compound that inhibits mRNA capping in an amount effective to reduce the viability of the cell (or inducing cell apoptosis).

As used herein, inhibiting mRNA capping includes inhibition of: (i) RNGTT and/or RNMT; and/or (ii) the synthesis of guanosine monophosphate (GMP), e.g., by inhibiting IMPDH1 and/or IMPDH2.

In some embodiments of any of the methods described herein, the cell is characterized by increased mTOR expression and/or increased mTOR activity, e.g., relative to a normal cell of the same histological type.

In some embodiments of any of the methods described herein, the cell is characterized as having one or more mutations in the TSC1 gene, the TSC2 gene, or both the TSC1 and TSC2 genes.

In some embodiments, any of the methods described herein can include determining whether a cell exhibits increased expression of mTOR. In some embodiments, any of the methods described herein can include determining whether a cell exhibits increased mTOR activity.

In some embodiments, any of the methods described herein can include determining whether a cell comprises a mutation in TSC1 or TSC2.

In some embodiments of any of the methods described herein, the one or more mutations in TSC1 or TSC2 result in reduced tumor suppressor activity of the Tumor Suppressor Complex (TSC). In some embodiments of any of the methods described herein, the one or more mutations in TSC1 or TSC2 are associated with increased mTOR expression or increased mTOR activity.

In some embodiments of any of the methods described herein, the cell is a mammalian cell (e.g., a rodent cell, a non-human primate cell, or a human cell). In some embodiments, the cell is one obtained from a subject having a proliferative disorder. In some embodiments, the cell is obtained from a subject having a cancer.

In some embodiments, the methods described herein are in vitro methods.

In some embodiments, the methods described herein are ex vivo methods.

In some embodiments, the methods described herein are in vivo methods, e.g., methods for inhibiting cell proliferation, methods for reducing cell viability, and/or methods for inhibiting cell growth in a subject by administering an effective amount of a compound described herein.

In yet another aspect, the disclosure features a method for treating a subject having a cell proliferative disorder characterized by proliferating cells that: (i) overexpress mTOR or (ii) have increased mTOR complex 1 (mTORC1) activity. The method comprises administering to the subject a compound that inhibits IMPDH (IMPDH1 or IMPDH2), RNGTT, RNMT, Cdk11, Cdk9, CCNT1, CCND3, Cyclin L1, or Cyclin L2 in an amount effective to treat the cell proliferative disorder.

In some embodiments of any of the methods described herein, the proliferating cells comprise at least one mutation in one or both of the TSC1 and TSC2 genes.

In another aspect, the disclosure features a method for treating a subject having a cell proliferative disorder, the method comprising administering to the subject a compound that inhibits IMPDH (IMPDH1 or IMPDH2), RNGTT, RNMT, Cdk11, Cdk9, CCNT1, CCND3, Cyclin L1, or Cyclin L2 in an amount effective to treat the cell proliferative disorder, wherein the subject has been identified as having a cell proliferative disorder characterized by proliferating cells that: (i) overexpress mTOR or (ii) have increased mTOR complex 1 (mTORC1) activity.

In another aspect, the disclosure features a method for treating a subject having a proliferative disorder characterized in that one or both of the TSC1 and TSC2 genes are mutated, which method comprises administering to the subject a compound that inhibits IMPDH (IMPDH1 or IMPDH2), RNGTT, RNMT, Cdk11, Cdk9, CCNT1, CCND3, Cyclin L1, or Cyclin L2, in an amount effective to treat the cell proliferative disorder.

In yet another aspect, the disclosure features a method for treating a subject having a proliferative disorder. The method comprises administering to the subject a compound that inhibits IMPDH (IMPDH1 or IMPDH2), RNGTT, RNMT, Cdk11, Cdk9, CCNT1, CCND3, Cyclin L1, or Cyclin L2 in an amount effective to treat the cell proliferative disorder, wherein the subject has been identified as having a cell proliferative disorder characterized in that one or both of the TSC1 and TSC2 genes are mutated.

In some embodiments of any of the methods described herein, the cell proliferative disorder is a cancer, such as, but not limited to, a lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer.

In some embodiments of any of the methods described herein, the cell proliferative disorder is tuberous sclerosis complex, lymphangioleiomyomatosis, a PTEN mutant hamartoma syndrome, Peutz Jeghers syndrome, Familial Adenomatous Polyposis, or neurofibromatosis type 1. The PTEN mutant hamartoma syndrome can be, e.g., Cowden disease, Proteus disease, Lhermitte-Duclos disease, or Bannayan-Riley-Ruvalcaba syndrome.

In yet another aspect, the disclosure features a method for treating a subject having an autoimmune or inflammatory disorder. The method comprises administering to the subject a compound that inhibits IMPDH (IMPDH1 or IMPDH2), RNGTT, RNMT, Cdk11, Cdk9, CCNT1, CCND3, Cyclin L1, or Cyclin L2 in an amount effective to treat the autoimmune or inflammatory disorder. In some embodiments of any of the methods described herein, the inflammatory or autoimmune disorder can be, e.g., osteoarthritis, Rheumatoid arthritis (RA), spondyloarhropathies, POEMS syndrome, Crohn's disease, multicentric Castleman's disease, systemic lupus erythematosus (SLE), multiple sclerosis (MS), muscular dystrophy (MD), insulin-dependent diabetes mellitus (IDDM), dermatomyositis, polymyositis, inflammatory neuropathies such as Guillain Barre syndrome, vasculitis such as Wegener's granulomatosus, polyarteritis nodosa, polymyalgia rheumatica, temporal arteritis, Sjogren's syndrome, Bechet's disease, Churg-Strauss syndrome, or Takayasu's arteritis.

In some embodiments of any of the methods described herein, the compound binds to IMPDH (IMPDH1 or IMPDH2), RNGTT, RNMT, Cdk11, Cdk9, CCNT1, CCND3, Cyclin L1, or Cyclin L2. In some embodiments of any of the methods described herein, the compound inhibits the activity of IMPDH (IMPDH1 or IMPDH2), RNGTT, RNMT, Cdk11, Cdk9, CCNT1, CCND3, Cyclin L1, or Cyclin L2. In some embodiments of any of the methods described herein, the compound binds to and inhibits the activity of RNGTT, RNMT, Cdk11, Cdk9, CCNT1, CCND3, Cyclin L1, or Cyclin L2.

In some embodiments of any of the methods described herein, the compound can be, e.g., a small molecule, a macrocycle compound, a polypeptide, a nucleic acid, or a nucleic acid analog.

In some embodiments of any of the methods described herein, the compound reduces the expression or stability of an mRNA encoding IMPDH (IMPDH1 or IMPDH2), RNGTT, RNMT, Cdk11, Cdk9, CCNT1, CCND3, Cyclin L1, or Cyclin L2 protein. In some embodiments of any of the methods described herein, the compound can be, e.g., an antisense oligonucleotide, an siRNA, an shRNA, or a ribozyme.

In some embodiments, any of the methods described herein can comprise determining whether the proliferating cells overexpress mTOR or have increased mTOR complex 1 (mTORC1) activity.

In some embodiments, any of the methods described herein comprise, prior to administering the compound to the subject, requesting the results of a test that determined whether the proliferating cells overexpress mTOR or have increased mTOR complex 1 (mTORC1) activity.

In some embodiments, any of the methods described herein comprise determining whether the proliferating cells comprise at least one mutation in one or both of the TSC1 and TSC2 genes.

In some embodiments, any of the methods described herein comprise, prior to administering the compound to the subject, requesting the results of a test that determined whether the proliferating cells comprise at least one mutation in one or both of the TSC1 and TSC2 genes.

In another aspect, the disclosure features a method for treating a subject having a proliferative disorder characterized in that one or both of TSC1 and TSC2 are mutated, the method comprising administering to the subject a compound that inhibits IMPDH (IMPDH1 or IMPDH2), RNGTT, RNMT, Cdk11, or CCNT1, in an amount effective to treat the cell proliferative disorder.

In some embodiments of any of the methods described herein, the compound binds to and inhibits the activity of IMPDH (IMPDH1 or IMPDH2), RNGTT, RNMT, Cdk11, or CCNT1. In some embodiments, the compound can be, e.g., a small molecule, a macrocycle compound, a polypeptide, a nucleic acid, or a nucleic acid analog.

In some embodiments of any of the methods described herein, the compound reduces the expression or stability of an mRNA encoding IMPDH (IMPDH1 or IMPDH2), RNGTT, RNMT, Cdk11, or CCNT1 protein. The compound ca be, e.g., an antisense oligonucleotide, an siRNA, an shRNA, or a ribozyme.

In some embodiments, any of the methods described herein can further comprise determining whether the proliferating cells comprise at least one mutation in one or both of the TSC1 and TSC2 genes.

In some embodiments, any of the methods described herein can comprise, prior to administering the compound to the subject, requesting the results of a test that determined whether the proliferating cells comprise at least one mutation in one or both of the TSC1 and TSC2 genes.

In another aspect, the disclosure features a method for treating a subject having a proliferative disorder. The method comprises administering to the subject a compound that inhibits mRNA capping in an amount effective to treat the cell proliferative disorder. In some embodiments, the compound inhibits the expression or activity of RNGTT or RNMT. In some embodiments, the compound binds to and inhibits the activity of RNGTT or RNMT. In some embodiments, the compound can be, e.g., a small molecule, a macrocycle compound, a polypeptide, a nucleic acid, or a nucleic acid analog. In some embodiments, the compound reduces the expression or stability of an mRNA encoding RNGTT protein or RNMT protein. In some embodiments, the compound can be, e.g., an antisense oligonucleotide, an siRNA, an shRNA, or a ribozyme.

In some embodiments of any of the methods described herein, the cell proliferative disorder is a cancer. The cancer can be, e.g., a lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer.

In some embodiments of any of the methods described herein, the cell proliferative disorder is tuberous sclerosis complex, lymphangioleiomyomatosis, a PTEN mutant hamartoma syndrome, Peutz Jeghers syndrome, Familial Adenomatous Polyposis, or neurofibromatosis type 1. The PTEN mutant hamartoma syndrome can be, e.g., Cowden disease, Proteus disease, Lhermitte-Duclos disease, or Bannayan-Riley-Ruvalcaba syndrome.

In some embodiments of any of the methods described herein, the subject is a human.

“Polypeptide,” “peptide,” and “protein” are used interchangeably and mean any peptide-linked chain of amino acids, regardless of length or post-translational modification. As noted below, the polypeptides described herein can be, e.g., wild-type proteins, functional fragments of the wild-type proteins, or variants of the wild-type proteins or fragments.

As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST software. Appropriate parameters for measuring alignment, including any algorithms needed to achieve maximal alignment over the full-length of the sequences being compared can be determined by known methods.

Unless otherwise defined, all technical and scientific terms used herein have the same meaning as commonly understood by one of ordinary skill in the art to which this disclosure pertains. In case of conflict, the present document, including definitions, will control. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

Other features and advantages of the present disclosure, e.g., methods for treating a proliferative disorder, will be apparent from the following description, the examples, and from the claims.

### BRIEF DESCRIPTION OF THE DRAWINGS

FIG. 1 comprises three panels: A, B, and C. Panel A is a bar graph showing relative mutation rates from 75 sgRNAs used to target a single sequence cloned into a luciferase reporter. Mutation rate is calculated as 1/Firefly luciferase activity normalized to Renilla luciferase activity to control for differential transfection efficiency. Bars show mean relative mutation rates from three biological replicates using sgRNAs with 0 mismatches (blue bar), 1 mismatch (grey bars), 2 mismatches (green bars), ≥3 mismatches (black bars) or in the absence of sgRNA (red bar). Dashes indicate nucleotides that are matched between sgRNA and the target sequence. Crosses indicate the position of mismatches. Panel B is a table showing enrichment p-values of each nucleotide in each position amongst high efficiency sgRNAs. Panel C is a pair of dot plots depicting the validation of efficiency scores generated using the matrix shown in B by correlating score (horizontal axis) with efficiency (vertical axis) from two independent publications.

FIG. 2 comprises six panels: A, B, C, D, E, and F. Panel A is a table showing survival rates of single S2R+ cells seeded into different media formulations. ‘Clones’ represents the number of seeded samples that produced viable populations of cells after three weeks. Schneider's media was supplemented with FBS at the concentrations indicated and was preconditioned using S2R+ cells where indicated (see Methods). Panel B is a line graph depicting the high resolution melt analysis (HRMA) results for single S2R+ cells from a population four days after treatment with CRISPR targeting the yellow gene. The graph shows the difference in fluorescence between each sample and a mean control curve against temperature. Panel C is a schematic for a workflow showing the major steps required to generate mutant cell lines. Panel D: provides schematics of the STAT92E and Ligase 4 (Lig4) genes. UTRs are represented by thin black boxes, coding exons by thick black boxes and introns by black lines. Arrows superimposed on introns indicate the direction of transcription. CRISPR target sites for each gene are shown the by grey arrows. Panel E is a bar graph showing relative Firefly luciferase activity normalized to Renilla luciferase activity for either wild-type or STAT92E mutant cells in the presence (red bars) or absence (blue bars) of JAK STAT pathway activation (upd ligand expression) and with activation in the presence of two different dsRNAs targeting STAT92E (green and purple bars). Bars show the mean from two biological replicates and error bars represent standard error of the mean. Panel F is a bar graph showing the percentage of cells expressing GFP following CRISPR induced recombination to insert GFP into the indicated genes. Results show a comparison between wild-type S2R+ cells (blue bars) and Lig4 mutant cells (red bars).

FIG. 3 comprises several panels, A to H. Panel A provides the schematics of the TSC1 and TSC2 genes. Details are as described for FIG. 2D, above. Panels B, C, and D are photographs of representative fields from wild-type (B), TSC1 mutant (C) or TSC2 mutant (D) cell lines. All images were taken at the same magnification and using the same settings. Scale bar represents 50 μm. Panel E is a graph showing frequency of cell sizes for the cell lines indicated, divided into low diameter′ (grey bars) or ‘high diameter’ (black bars) using a cutoff at which the majority of wild-type cells fall into the low diameter′ category. Panel F is a bar graph showing relative rates of population growth for the cell lines indicated in either complete media (10% FBS—blue bars), under partial starvation conditions (1% FBS red bars) or complete starvation conditions (no FBS—green bars). Note that these values represent a combination of cell growth and proliferation. Bars show the mean of at least 24 samples and error bars represent standard error of the mean. Panel G is a bar graph depicting the quantification of p-S6K levels for the cell lines indicated measured using in-cell westerns. Bars represent mean fold change in p-S6K levels normalized to Tubulin levels for 4 replicates in each case. Error bars represent standard error of the mean and asterisks indicate significant differences from control (p≤0.01) based on t-tests. Panel H is a bar graph indicating the fold enrichment of the indicated GO categories in phosphoproteomic data from TSC1 and TSC2 mutant cells compared to wild-type. All samples are enriched with p-values less than 0.05.

FIG. 4 comprises a series of panels, A to D. Panel A is a schematic of the synthetic screening approach. Panel B is a scatter plot showing results of screens in Drosophila TSC1 and TSC2 mutant cell lines. dsRNAs that showed significant changes in wild-type cells are not shown on the graph. Points indicate the Z-score from three replicate screens in TSC1 cells (horizontal axis) and TSC2 cells (Vertical axis). Dots represent non-hits (black circles), TSC1 specific hits (red circles), TSC2 specific hits (blue circles) and hits from TSC1 and TSC2 cells (purple crosses). The three genes showing synthetic lethal interactions with both TSC1 and TSC2 are labeled. In addition, results for eIF3 are plotted on the same graph for comparison (purple circle). Panel C is a box and whisker plot depicting population growth assays in TSC2 deficient or wild-type MEFs treated with the siRNAs indicated. All differences between TSC deficient and wild-type cells are significant (p<0.05). Boxplots represent median (thick black lines), interquartile range (boxes) and min/max (error bars) for the genes indicated in TSC2 deficient or wild-type backgrounds. The vertical axis represents change in ATP levels after 48 hours of culture relative to cells treated with control siRNA measured using CellTiter glo assays. Panel D is another box and whisker plot depicting population growth assays in TSC2 deficient AML cells. Boxplots are as described in D. All differences between TSC deficient and wild-type cells are significant (p<0.05).

FIG. 5 is a bar graph showing relative mutation rates from 75 sgRNAs used to target a single sequence in the yellow gene. Mutation rate is calculated as integrated area between each experimental HRM curve and a mean control curve. Each bar represents the mean relative mutation rate from three biological replicates using sgRNAs with 0 mismatches (blue bar), 1 mismatch (grey bars), 2 mismatches (green bars), ≥3 mismatches (black bars) or in the absence of sgRNA (red bar). Dashes indicate nucleotides that are matched between sgRNA and the target sequence. Crosses indicate the position of mismatches. Error bars indicate standard error of the mean.

FIG. 6 is a series of graphs as panels A, B, and C, comparing the sgRNA mutagenesis efficiency to GC content considering the final 4 nucleotides (Panel A), the final 6 nucleotides (Panel B) or the whole sgRNA sequence (Panel C).

FIG. 7 depicts the sequences from 8 individual cells transfected with CRISPR reagents targeting the yellow gene. Samples are numbered 1 to 8, with a minimum of 5 sequence reads shown for each. The top row shows wild-type sequence.

FIG. 8 depicts the sequencing results for at least 20 clones from TSC1 or TSC2 mutant cell lines as indicated. Asterisks indicate wild-type sequence.

FIG. 9 is a series of panels, panels A-E showing TSC2 loss confers sensitivity to CCNT1, RNGTT, or CDK11 loss of function. Panel A is a schematic of the methods disclosed herein. Panels B-E are a series of line graphs showing various compounds selectively reduce proliferation of Tsc2−/− MEFs relative to Tsc2+/+ MEFs. Panel B represents data corresponding to the CDK9/CDK11 inhibitor JWD07 (5 μM). Panel C represents data corresponding to the CDK2/CDK9/CDK11 inhibitor AT7915 (2 μM). Panel D represents data corresponding to the RNGTT/IMPDH inhibitor Mizoribine (2.5 μM). Panel E represents data corresponding to the mTORC1 inhibitor Rapamycin (20 nM). As the data indicates, these compounds are superior to rapamycin for selective effects on cell viability in Tsc2−/− cells and exert a cytotoxic effect on these cells.

### DETAILED DESCRIPTION

The present disclosure provides, among other things, compounds that inhibit the expression or activity of gene products having a synthetic lethal interaction with TSC1 and/or TSC2. Also provided are applications, such as therapeutic and diagnostic methods, in which the compounds are useful. While in no way intended to be limiting, exemplary agents, compositions (e.g., pharmaceutical compositions and formulations), and methods for preparing and using these agents and compositions are elaborated on below.

### Compounds

The disclosure features agents that inhibit one or more gene products having a synthetic lethal interaction with TSC1 and/or TSC2. Inhibition of a gene or gene product (e.g., IMPDH1, IMPDH2, RNGTT, RNMT, Cdk11, Cdk9, CCNT1, CCND3, Cyclin L1, or Cyclin L2) can be inhibition of: (i) the transcription of a coding sequence for one of the gene products, (ii) the translation of an mRNA encoding one of the gene products, (iii) the stability of an mRNA encoding one of the gene products, (iv) the intracellular trafficking of one of the gene products, (v) the stability of the gene products (i.e., protein stability or turnover), (vi) the interaction of the gene product with another protein (e.g., inhibition of the interaction between Cdk and cyclin), and/or (vii) the activity of one of the gene products (e.g., inhibition of the kinase activity of a cyclin dependent kinase). The compound can be, e.g., a small molecule, a nucleic acid or nucleic acid analog, a peptidomimetic, a polypeptide, a macrocycle compound, or a macromolecule that is not a nucleic acid or a protein. These compounds include, but are not limited to, small organic molecules, RNA aptamers, L-RNA aptamers, Spiegelmers, nucleobase, nucleoside, nucleotide, antisense compounds, double stranded RNA, small interfering RNA (siRNA), locked nucleic acid inhibitors, peptide nucleic acid inhibitors, and/or analogs of any of the foregoing. In some embodiments, a compound may be a protein or protein fragment.

As used herein, the term “inhibiting” and grammatical equivalents thereof refer to a decrease, limiting, and/or blocking of a particular action, function, or interaction. In one embodiment, the term refers to reducing the level of a given output or parameter to a quantity which is at least 10%, 15%, 20%, 25%, 30%, 35%, 40%, 45%, 50%, 55%, 60%, 65%, 70%, 75%, 80%, 85%, 90%, 95%, 99% or less than the quantity in a corresponding control. A reduced level of a given output or parameter need not, although it may, mean an absolute absence of the output or parameter. The disclosure does not require, and is not limited to, methods that wholly eliminate the output or parameter.

As used herein, the term “interaction”, when referring to an interaction between two molecules, refers to the physical contact (e.g., binding) of the molecules with one another. Generally, such an interaction results in an activity (which produces a biological effect) of one or both of said molecules. To inhibit such an interaction results in the disruption of the activity of one or more molecules involved in the interaction.

In some embodiments, the compound described herein has an IC₅₀ (e.g., against the gene product as measured in an in vitro assay) of less than 1 μM (e.g., less than 900, 800, 700, 600, 500, 400, 300, 200, 100, 50, 25, 10, 5, 1, 0.9, 0.8, 0.7, 0.6, 0.5, 0.4, 0.3, 0.2, or 0.1 nM). In some embodiments, the compound described herein has an EC₅₀ (e.g., in cell-based assays, e.g., proliferation assays) of less than 10 μM (e.g., less than 9, 8, 7, 6, 5, 4, 3, 2, or 1 μM, or less than 900, 800, 700, 600, 500, 400, 300, 200, 100, 50, 25, 10, 5, 1, 0.9, 0.8, 0.7, 0.6, 0.5, 0.4, 0.3, 0.2, or 0.1 nM). In some embodiments, an compound specifically binds to a gene product of interest. The terms “specific binding,” “specifically binds,” and like grammatical terms, as used herein, refer to two molecules forming a complex that is relatively stable under physiologic conditions. Typically, binding is considered specific when the association constant (kₐ) is higher than 10⁶ M⁻¹S⁻¹. Thus, a compound can specifically bind to a protein with a kₐ of at least (or greater than) 10⁶ (e.g., at least or greater than 10⁷, 10⁸, 10⁹, 10¹⁰, 10¹¹, 10¹², 10¹³, 10¹⁴, or 10¹⁵ or higher) In some embodiments, a compound described herein has a dissociation constant (k_{d}) of less than or equal to 10⁻³ (e.g., 8×10⁻⁴, 5×10⁻⁴, 2×10⁻⁴, 10⁻⁴, or 10⁻⁵) s⁻¹.

In some embodiments, a compound described herein has a K_{D} of less than 10⁻⁸, 10⁻⁹, 10¹⁰, 10¹¹, or 10¹² M. The equilibrium constant K_{D} is the ratio of the kinetic rate constants—k_{d}/kₐ. In some embodiments, a compound described herein has a K_{D} for its target protein of less than 1×10⁻⁹ M.

Small Molecules and Peptides

“Small molecule” as used herein, is meant to refer to an agent, which has a molecular weight of less than about 6 kDa and most preferably less than about 2.5 kDa. Many pharmaceutical companies have extensive libraries of chemical and/or biological mixtures comprising arrays of small molecules, often fungal, bacterial, or algal extracts, which can be screened with any of the assays of the application. This application contemplates using, among other things, small chemical libraries, peptide libraries, or collections of natural products. Tan et al. described a library with over two million synthetic compounds that is compatible with miniaturized cell-based assays (J Am Chem Soc (1998) 120:8565-8566), It is within the scope of this application that such a library may be used to screen for inhibitors (e.g., kinase inhibitors) of any one of the gene products described herein, e.g., cyclin dependent kinases. There are numerous commercially available compound libraries, such as the Chembridge DIVERSet. Libraries are also e from academic investigators, such as the Diversity set from the NCI developmental therapeutics program. Rational drug design may also be employed.

Compounds useful in the methods of the present invention may be obtained from any available source, including systematic libraries of natural and/or synthetic compounds. Compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994 J. Med. Chem. 37:2678-85, which is expressly incorporated by reference); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des, 12:145, which is expressly incorporated by reference).

Examples of methods for the synthesis of molecular libraries can be found in the art. for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994), J. Med. Chem, 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233, each of which is expressly incorporated by reference.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci, 87:6378-6382; Felici, 1991, J. Mol. Biol. 222:301-310; Ladner, supra., each of which is expressly incorporated by reference).

Peptidomimetics can be compounds in which at least a portion of a subject polypeptide is modified, and the three dimensional structure of the peptidomimetic remains substantially the same as that of the subject polypeptide. Peptidomimetics may be analogues of a subject polypeptide of the disclosure that are, themselves, polypeptides containing one or more substitutions or other modifications within the subject polypeptide sequence. Alternatively, at least a portion of the subject polypeptide sequence may be replaced with a non-peptide structure, such that the three-dimensional structure of the subject polypeptide is substantially retained. In other words, one, two or three amino acid residues within the subject polypeptide sequence may be replaced by a non-peptide structure. In addition, other peptide portions of the subject polypeptide may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of humans or animals. It should be noted that peptidomimetics may or may not have similar two-dimensional chemical structures, but share common three-dimensional structural features and geometry. Each peptidomimetic may further have one or more unique additional binding elements.

Nucleic Acids

Nucleic acid inhibitors can be used to decrease expression of an endogenous gene encoding one of the gene products described herein. The nucleic acid antagonist can be; e.g., an siRNA, a dsRNA, a ribozyme, a triple-helix former, an aptamer, or an antisense nucleic acid. siRNAs are small double stranded RNAs (dsRNAs) that optionally include overhangs, For example, the duplex region of an siRNA is about 18 to 25 nucleotides in length, e.g., about 19, 20, 21, 22, 23, or 24 nucleotides in length. The siRNA sequences can be, in some embodiments, exactly complementary to the target mRNA. dsRNAs and siRNAs in particular can be used to silence gene expression in mammalian cells (e.g., human cells). See, e.g.; Clemens et al. (2000) Proc Natl Acad Sci USA 97:6499-6503; Billy et al, (2001) Proc Natl Acad Sci USA 98:14428-14433; Elbashir et al. (2001) Nature 411:494-8; Yang et al. (2002) Proc Natl Acrid Sci USA 99:9942-9947, and U.S. Patent Application Publication Nos, 20030166282, 20030143204, 20040038278, and 20030224432. Antisense agents can include, for example, from about 8 to about 80 nucleobases (i.e. from about 8 to about 80 nucleotides), e.g., about 8 to about 50 nucleobases, or about 12 to about 30 nucleobases. Antisense compounds include ribozymes, external guide sequence (EGS) oligonucleotides (oligozytnes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically, hybridizable, An oligonucleotide is specifically hybridizable when binding of the oligonucleotide to the target interferes with the normal function of the target molecule to cause a loss of utility, and there is a sufficient degree of complementarily to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

siRNA molecules can be prepared by chemical synthesis, in vitro transcription, or digestion of long dsRNA by Rnase III or Dicer. These can be introduced into cells by transfection, electroporation, intracellular infection or other methods known in the art. See, for example; each of which is expressly incorporated by reference: Hannon, G J, 2002, RNA Interference, Nature 418: 244-251; Bernstein E et al., 2002, The rest is silence. RNA 7: 1509-1521; Hutvagner G et al., RNAi: Nature abhors a double-strand, Cur. Open. Genetics & Development 12: 225-232; Brummelkamp, 2002, A system for stable expression of short interfering RINAs in mammalian cells, Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002).1,16-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer 1, and Engelke D R. (2.002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells, Proc. Natl. Acad. Sci. USA 99(9):6047-6052, PCT publications WO2006/066048 and WO2009/029688, U.S. published application U.S. 2009/0123426, each of which is incorporated by reference in its entirety.

Hybridization of antisense oligonucleotides with mRNA can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA. Exemplary antisense compounds include DNA or RNA sequences that specifically hybridize to the target nucleic acid, e.g., the mRNA encoding one of the gene products described herein. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C₅-propynyl pyrimidines such as Cs-propynylcytosine and C₅-propynyluracil. Other suitable modified nucleobases include, e.g., 7-substituted-8-aza-7-deazapurines and 7-substituted-7-deazapurines such as, for example, 7-iodo-7-deazapurines, 7-cyano-7-deazapurines; 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. See, e.g., U.S. Pat. Nos. 4,987,071; 5,116,742; and U.S. Pat. No. 5,093,246; “Antisense RNA and DNA,” D. A. Melton, Ed., Cold Spring Harbor Laboratory, Cold Spring Harbor, N.Y. (1988); Haselhoff and Gerlach (1988) Nature 334:585-59; Helene, C. (1991) Anticancer Drug D 6:569-84; Helene (1992) Ann NY Acad Sci 660:27-36; and Maher (1992) Bioassays 14:807-1.5.

Aptamers are short oligonucleotide sequences that can be used to recognize and specifically bind almost any molecule, including cell surface proteins. The systematic evolution of ligands by exponential enrichment (SELEX) process is powerful and can be used to readily identify such aptamers. Aptamers can be made for a wide range of proteins of importance for therapy and diagnostics, such as growth factors and cell surface antigens. These oligonucleotides bind their targets with similar affinities and specificities as antibodies do (see, e.g., Ulrich (2006) Handb Exp Pharmacol 173:305-326).

Antisense or RNA interference molecules can be delivered in vitro to cells or in vivo. Typical delivery means known in the art can be used. Any mode of delivery can be used without limitation, including: intravenous, intramuscular, intraperitoneal, intraarterial, local delivery during surgery, endoscopic, or subcutaneous. Vectors can be selected for desirable properties for any particular application. Vectors can be viral, bacterial or plasmid. Adenoviral vectors are useful in this regard. Tissue-specific, cell-type specific, or otherwise regulatable promoters can be used to control the transcription of the inhibitory polynucleotide molecules. Non-viral carriers such as liposomes or nanospheres can also be used.

In the present methods, a RNA interference molecule or an RNA interference encoding oligonucleotide can be administered to the subject, for example, as naked RNA, in combination with a delivery reagent, and/or as a nucleic acid comprising sequences that express the siRNA or shRNA molecules. In some embodiments the nucleic acid comprising sequences that express the siRNA or shRNA molecules are delivered within vectors, e.g. plasmid, viral and bacterial vectors. Any nucleic acid delivery method known in the art can be used in the present invention. Suitable delivery reagents include, but are not limited to, e.g., the Minis Transit TKO lipophilic reagent; lipofectin; lipofectamine; cellfectin; polycations (e.g., polylysine), atelocollagen, nanoplexes and liposomes.

The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety.

In some embodiments of the invention, liposomes are used to deliver an inhibitory oligonucleotide to a subject. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In an embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example solid tumors, will efficiently accumulate these liposomes; see Gabizon, et al. (1988), Proc. Natl. Acad. Sci., USA, 18:6949-53, which is expressly incorporated by reference. In addition, the reduced uptake by the RES lowers the toxicity of stealth liposomes by preventing significant accumulation of the liposomes in the liver and spleen.

The nucleotide sequences encoding the gene products described herein (from multiple species, including human), from which exemplary nucleic acid inhibitors can be designed, are known in the art and are publicly available. For example, an exemplary nucleotide sequence encoding human Cdk9 is as follows:

| | |
|---|---|
| 1 | atggcaaagc agtacgactc ggtggagtgc cctttttgtg atgaagtttc caaatacgag |
| 61 | aagctcgcca agatcggcca aggcaccttc ggggaggtgt tcaaggccag gcaccgcaag |
| 121 | accggccaga aggtggctct gaagaaggtg ctgatggaaa acgagaagga ggggttcccc |
| 181 | attacagcct tgcgggagat caagatcctt cagcttctaa aacacgagaa tgtggtcaac |
| 241 | ttgattgaga tttgtcgaac caaagcttcc ccctataacc gctgcaaggg tagtatatac |
| 301 | ctggtgttcg acttctgcga gcatgacctt gctgggctgt tgagcaatgt tttggtcaag |
| 361 | ttcacgctgt ctgagatcaa gagggtgatg cagatgctgc ttaacggcct ctactacatc |
| 421 | cacagaaaca agatcctgca tagggacatg aaggctgcta atgtgcttat cactcgtgat |
| 481 | ggggtcctga agctggcaga ctttgggctg gcccgggcct tcagcctggc caagaacagc |
| 541 | cagcccaacc gctacaccaa ccgtgtggtg acactctggt accggccccc ggagctgttg |
| 601 | ctcggggagc gggactacgg cccccccatt gacctgtggg gtgctgggtg catcatggca |
| 661 | gagatgtgga cccgcagccc catcatgcag ggcaacacgg agcagcacca actcgccctc |
| 721 | atcagtcagc tctgcggctc catcacccct gaggtgtggc caaacgtgga caactatgag |
| 781 | ctgtacgaaa agctggagct ggtcaagggc cagaagcgga aggtgaagga caggctgaag |
| 841 | gcctatgtgc gtgacccata cgcactggac ctcatcgaca agctgctggt gctggaccct |
| 901 | gcccagcgca tcgacagcga tgacgccctc aaccacgact tcttctggtc cgaccccatg |
| 961 | ccctccgacc tcaagggcat gctctccacc cacctgacgt ccatgttcga gtacttggca |
| 1021 | ccaccgcgcc ggaagggcag ccagatcacc cagcagtcca ccaaccaga tcgcaatccc |
| 1081 | gccaccacca accagacgga gtttgagcgc gtcttctga |

(SEQ ID NO:1; NCBI Reference No. NM_001261.3). An exemplary nucleotide sequence encoding human Cdk11(B) is as follows:

| | |
|---|---|
| 1 | atgggtgatg aaaaggactc ttggaaagtg aaaactttag atgaaattct tcaggaaaag |
| 61 | aaacgaagga aggaacaaga ggagaaagca gagataaaac gcttaaaaaa ttctgatgac |
| 121 | cgggattcca agcgggattc ccttgaggag ggggagctga gagatcaccg catggagatc |
| 181 | acaataagga actccccgta tagaagagaa gactctatgg aagacagagg agaagaagat |
| 241 | gattctttgg ccatcaaacc accccagcaa atgtctcgga aagaaaaagc tcatcacaga |
| 301 | aaagatgaaa agagaaaaga gaaacgtagg catcgtagcc attcagcaga aggggggaag |
| 361 | catgctagag tgaaagaaaa agaaagagag cacgaacgtc ggaaacggca tcgagaagaa |
| 421 | caggataaag ctcgccggga atgggaaaga cagaagagaa gggagatggc aagggagcat |
| 481 | tccaggagag aaagggaccg cttggagcag ttagaaagga agcgggagcg ggagcgcaag |
| 541 | atgcgggagc agcagaagga gcagcgggag cagaaggagc gcgagcggcg ggcagaggag |
| 601 | cggcgcaagg agcgggaggc ccgcagggaa gtgtctgcac atcaccgaac gatgagagag |
| 661 | gactacagcg acaaagtgaa agccagccac tggagtcgca gcccgcctcg gccgccgcgg |
| 721 | gagcggttcg agttgggaga cggccggaag ccaggtgagg ccaggccggc gcctgcgcag |
| 781 | aagccagcac agttaaaaga agagaaaatg gaagaaaggg acctgctgtc cgacttacag |
| 841 | gacatcagcg acagcgagag gaagaccagc tcggccgagt cctcgtcagc ggaatcaggc |
| 901 | tcaggttctg aggaagaaga ggaggaggag gaagaggagg aggaggaagg gagcaccagt |
| 961 | gaagaatcag aggaggagga ggaggaagag gaagaggagg aggaggagac cggcagcaac |
| 1021 | tctgaggagg catcagagca gtctgccgaa gaagtaagtg aggaagaaat gagtgaagat |
| 1081 | gaagaacgag aaaatgaaaa ccacctcttg gttgttccag agtcacggtt cgaccgagat |
| 1141 | tccggggaga gtgaagaagc agaggaagaa gtgggtgagg gaacgccgca gagcagcgcc |
| 1201 | ctgacagagg gcgactatgt gcccgactcc cctgccctgt cgcccatcga gctcaagcag |
| 1261 | gagctgccca agtacctgcc ggccctgcag ggctgccgga gcgtcgagga gttccagtgc |
| 1321 | ctgaacagga tcgaggaggg cacctatgga gtggtctaca gagcaaaaga caagaaaaca |
| 1381 | gatgaaattg tggctctaaa gcggctgaag atggagaagg agaaggaggg cttcccgatc |
| 1441 | acgtcgctga gggagatcaa caccatcctc aaggcccagc atcccaacat cgtcaccgtt |
| 1501 | agagagattg tggtgggcag caacatggac aagatctaca tcgtgatgaa ctatgtggag |
| 1561 | cacgacctca agagcctgat ggagaccatg aaacagccct tcctgccagg ggaggtgaag |
| 1621 | accctgatga tccagctgct gcgtggggtg aaacacctgc acgacaactg gatcctgcac |
| 1681 | cgtgacctca agacgtccaa cctgctgctg agccacgccg gcatcctcaa ggtgggtgac |
| 1741 | ttcgggctgg cgcgggagta cggatcccct ctgaaggcct acaccccggt cgtggtgacc |
| 1801 | ctgtggtacc gcgccccaga gctgctgctt ggtgccaagg aatactccac ggccgtggac |
| 1861 | atgtggtcag tgggttgcat cttcggggag ctgctgactc agaagcctct gttccccggg |
| 1921 | aagtcagaaa tcgatcagat caacaaggtg ttcaaggatc tggggacccc tagtgagaaa |
| 1981 | atctggcccg gctacagcga gctcccagca gtcaagaaga tgaccttcag cgagcacccc |
| 2041 | tacaacaacc tccgcaagcg cttcggggct ctgctctcag accagggctt cgacctcatg |
| 2101 | aacaagttcc tgacctactt ccccgggagg aggatcagcg ctgaggacgg cctcaagcat |
| 2161 | gagtatttcc gcgagacccc cctccccatc gacccctcca tgttccccac gtggcccgcc |
| 2221 | aagagcgagc agcagcgtgt gaagcggggc accagcccga ggccccctga gggaggcctg |
| 2281 | ggctacagcc agctgggtga cgacgacctg aaggagacgg gcttccacct taccaccacg |
| 2341 | aaccaggggg cctctgccgc gggccccggc ttcagcctca agttctga |

(SEQ ID NO:2; NCBI Reference No. NM_001787.2). An exemplary nucleotide sequence encoding human Cdk11A is as follows:

| | |
|---|---|
| 1 | atgggtgatg aaaaggactc ttggaaagtg aaaactttag atgaaattct tcaggaaaag |
| 61 | aaacgaagga aggaacaaga ggagaaagca gagataaaac gcttaaaaaa ttctgatgac |
| 121 | cgggattcca agcgggattc ccttgaggag ggggagctga gagatcactg catggagatc |
| 181 | acaataagga actccccgta tagaagagaa gactcaatgg aagacagagg agaagaagat |
| 241 | gattctttgg ccatcaaacc accccagcaa atgtctcgga aagaaaaagt tcatcacaga |
| 301 | aaagatgaaa agagaaaaga aaaatgtagg catcatagcc attcagcaga aggggggaag |
| 361 | catgctagag tgaaagaaag agagcacgaa cgtcggaaac gacatcgaga agaacaggat |
| 421 | aaagctcgcc gggaatggga aagacagaag agaagggaaa tggcaaggga gcattccagg |
| 481 | agagaaaggg accgcttgga gcagttagaa aggaagcggg agcgggagcg caagatgcgg |
| 541 | gagcagcaga aggagcagcg ggagcagaag gagcgcgagc ggcgggcgga ggagcggcgc |
| 601 | aaggagcggg aggcccgcag ggaagtgtct gcacatcacc gaacgatgag agaggactac |
| 661 | agcgacaaag tgaaagccag ccactggagt cgcagcccgc ctcggccgcc gcgggagcgg |
| 721 | ttcgagttgg gagacggccg gaagccagta aaagaagaga aaatggaaga aagggacctg |
| 781 | ctgtccgact tacaggacat cagcgacagc gagaggaaga ccagctcggc cgagtcctcg |
| 841 | tcagcggaat caggctcagg ttctgaggaa gaagaggagg aggaggaaga ggaggaggag |
| 901 | gaagggagca ccagtgaaga atcagaggag gaggaggagg aagaggaaga ggaggaggag |
| 961 | gagaccggca gcaactctga ggaggcatca gagcagtctg ccgaagaagt aagtgaggaa |
| 1021 | gaaatgagtg aagatgaaga acgagaaaat gaaaaccacc tcttggttgt tccagagtca |
| 1081 | cggttcgacc gagattccgg ggagagtgaa gaagcagagg aagaagtggg tgagggaacg |
| 1141 | ccgcagagca gcgccctgac agagggcgac tatgtgcccg actcccctgc cctgttgccc |
| 1201 | atcgagctca agcaggagct gcccaagtac ctgccggccc tgcagggctg ccggagcgtc |
| 1261 | gaggagttcc agtgcctgaa caggatcgag gagggcacct atggagtggt ctacagagca |
| 1321 | aaagacaaga aaacagatga aattgtggct ctaaagcggc tgaagatgga gaaggagaag |
| 1381 | gagggcttcc cgatcacgtc cctgagggag atcaacacca tcctcaaggc ccagcatccc |
| 1441 | aacattgtca ccgttagaga gattgtggtg ggcagcaaca tggacaagat ctacatcgtg |
| 1501 | atgaactacg tggagcacga cctcaagagc ctgatggaga ccatgaaaca gcccttcctg |
| 1561 | ccaggggagg tgaagaccct gatgatccag ctgctgcggg gggtgaaaca cctgcacgac |
| 1621 | aactggatcc tgcaccgtga cctcaagacg tccaacctgc tgctgagcca cgccggcatc |
| 1681 | ctcaaggtgg gtgattttgg gctggcgcgg gagtacggat cccctctgaa ggcctacacc |
| 1741 | ccggtcgtgg tgacccagtg gtaccgcgcc ccagagctgc tgcttggtgc caaggaatac |
| 1801 | tccacggccg tggacatgtg gtcagtgggc tgcatcttcg gggagctgct gactcagaag |
| 1861 | cctctgttcc ccgggaattc ggaaatcgat cagatcaaca aagtgttcaa ggagctgggg |
| 1921 | acccccagtg agaaaatctg gcccggctac agtgagctcc cagtagtcaa aaagatgacc |
| 1981 | ttcagcgagc acccctacaa caacctccgc aagcgcttcg gggctctgct ctcagaccag |
| 2041 | ggcttcgacc tcatgaacaa gttcctgacc tacttccccg ggaggaggat cagcgctgag |
| 2101 | gacggcctca agcatgagta tttccgcgag acccccctcc ccatcgaccc ctccatgttc |
| 2161 | cccacgtggc ccgccaagag cgagcagcag cgtgtgaagc ggggcaccag cccgaggccc |
| 2221 | cctgagggag gcctgggcta cagccagctg ggtgacgacg acctgaagga gacgggcttc |
| 2281 | caccttacca ccacgaacca gggggcctct gccgcgggcc ccggcttcag cctcaagttc |
| 2341 | tga |

(SEQ ID NO:12; NCBI Reference No. NM_024011.2).

An exemplary nucleotide sequence encoding human RNGTT is as follows:

| | |
|---|---|
| 1 | atggctcaca acaagatccc gccgcggtgg ctgaactgtc cccggcgcgg ccagccggtg |
| 61 | gcaggaagat tcttacctct gaagacaatg ttaggaccaa gatatgatag tcaagttgct |
| 121 | gaagaaaatc ggttccatcc cagcatgctc tcaaattacc taaagagcct aaaggttaaa |
| 181 | atgggcttgt tggtggacct gacaaatact tcaaggttct atgaccgaaa tgacatagaa |
| 241 | aaagaaggaa tcaaatatat aaaacttcag tgtaaaggac atggtgagtg ccctaccact |
| 301 | gagaatactg agacctttat tcgtctgtgt gagcggttta atgaaagaaa tccacctgaa |
| 361 | cttataggtg ttcattgtac tcatggcttc aatcgcactg gtttcctcat atgtgccttt |
| 421 | ttggtggaga aaatggattg gagtatcgaa gcagcagttg ctacttttgc ccaagccaga |
| 481 | ccaccaggaa tctacaaggg tgattatttg aaggaacttt ttcgtcggta tggtgacata |
| 541 | gaggaagcac cacccccacc tctattgcca gattggtgtt ttgaggatga tgaagacgaa |
| 601 | gatgaggatg aggatggaaa gaaggaatca gaacccgggt caagtgcttc ttttggcaaa |
| 661 | aggagaaaag aacggttaaa actgggcgct attttcttgg aaggtgttac tgttaaaggt |
| 721 | gtaactcaag taacaacaca accaaagtta ggagaggtac agcagaagtg tcatcaattc |
| 781 | tgtggctggg aagggtctgg attccctgga gcacagcctg tttccatgga caagcaaaat |
| 841 | attaaacttt tagacctgaa gccatacaaa gtaagctgga aagcagatgg tactcggtac |
| 901 | atgatgttga ttgatggcac aaatgaagtt tttatgattg atagagacaa ttcagtattt |
| 961 | catgtttcaa atctggaatt tccatttcgt aaagatcttc gtatgcattt atcaaatact |
| 1021 | ctcttggatg gcgagatgat tattgacaga gtaaatggac aggctgttcc tagatatttg |
| 1081 | atatatgaca taattaaatt caattcacag cccgttggag attgtgattt taatgttcgt |
| 1141 | ctgcagtgta tagaacgaga aattataagt cctcgacacg aaaaaatgaa gactgggctc |
| 1201 | attgacaaaa cacaggaacc atttagcgtc agaaataagc cgttttttga catctgtact |
| 1261 | tcaagaaagc tacttgaagg aaattttgcc aaagaagtga gccatgaaat ggatggactt |
| 1321 | atttttcagc ctactggaaa atacaaacct ggtcgatgtg atgatatttt gaaatggaag |
| 1381 | cctcccagtc tgaattctgt ggattttcgt ctaaaaataa caagaatggg aggagaaggg |
| 1441 | ttacttcctc agaatgttgg cctcctgtat gttggaggtt atgaaagacc ctttgcacaa |
| 1501 | atcaaggtga caaaagagct gaaacagtat gacaacaaaa ttatagaatg caaatttgag |
| 1561 | aacaacagct gggtcttcat gagacagaga acagacaaaa gttttcctaa tgcctacaac |
| 1621 | actgccatgg ctgtgtgtaa cagcatctca aaccctgtca ccaaggagat gctgtttgag |
| 1681 | ttcatcgaca gatgtactgc agcttctcaa ggacagaagc gaaaacatca tctggaccct |
| 1741 | gacacggagc tcatgccacc accacctccc aaaagaccac gccctttaac ctaa |

(SEQ ID NO:3; NCBI Reference No. NM_003800.4). An exemplary nucleotide sequence encoding human RNMT is as follows:

| | |
|---|---|
| 1 | atggcaaatt ctgcaaaagc agaagaatat gaaaagatgt ctcttgaaca ggcaaaagcg |
| 61 | tcagtgaatt ctgaaacaga gtcttcattc aatattaatg aaaacacaac agcttctggg |
| 121 | actgggcttt ctgaaaagac ttctgtctgt aggcaagtag acatagcaag aaagagaaaa |
| 181 | gagtttgaag atgatcttgt aaaggaaagt tctagttgtg ggaaagacac tccatccaag |
| 241 | aagagaaaac ttgatcctga aattgtccca gaggaaaaag attgtggtga tgctgaaggc |
| 301 | aattcaaaga aaagaaaaag agaaactgag gatgttccaa aagataaatc ttctactgga |
| 361 | gatggcactc aaaataagag aaaaatagca cttgaggatg ttcctgaaaa gcagaaaaat |
| 421 | ctggaagaag gacacagctc aacagtggct gcccattaca atgaacttca ggaagttggt |
| 481 | ttggagaagc gtagtcaaag tcgtattttt tacctaagaa actttaataa ttggatgaaa |
| 541 | agtgttctca ttggagaatt tttggaaaag gtacgacaga agaaaaaacg tgatatcact |
| 601 | gttttggacc tgggatgtgg taaaggtgga gatttgctga aatggaaaaa aggaagaatt |
| 661 | aacaagctag tttgtactga tattgccgat gtttctgtca aacagtgtca gcagcggtat |
| 721 | gaggacatga aaaatcgtcg tgatagtgaa tatattttca gtgcagaatt tataactgct |
| 781 | gacagctcaa aggaacttct gattgacaaa tttcgtgacc cacaaatgtg ttttgacatc |
| 841 | tgcagttgtc agtttgtctg tcattactca tttgagtctt atgagcaggc tgacatgatg |
| 901 | ctgagaaatg cgtgtgagag acttagccct gggggctatt ttattggtac tactcccaat |
| 961 | agctttgaat tgataagacg ccttgaagct tcagaaacag aatcatttgg aaatgaaata |
| 1021 | tatactgtga aatttcagaa gaaaggagat tatcctttat ttggctgcaa atatgacttc |
| 1081 | aacttggaag gtgttgtgga tgttcctgaa ttcttggtct attttccatt gctaaatgaa |
| 1141 | atggcaaaga agtacaatat gaaactagtc tacaaaaaaa catttctgga attctacgaa |
| 1201 | gaaaagatta agaacaatga aaataaaatg ctcttaaaac gaatgcaggc cttggagcca |
| 1261 | tatcctgcaa atgagagttc taaacttgtc tctgagaagg tggatgacta tgaacatgca |
| 1321 | gcaaagtaca tgaagaacag tcaagtaagg ttacctttgg gaaccttaag taaatcagaa |
| 1381 | tgggaagcta caagtattta cttggtgttt gcctttgaga aacagcagtg a |

(SEQ ID NO:4; NCBI Reference No. NM_003799.1). An exemplary nucleotide sequence encoding human CCNT1 is as follows:

| | |
|---|---|
| 1 | atggagggag agaggaagaa caacaacaaa cggtggtatt tcactcgaga acagctggaa |
| 61 | aatagcccat cccgtcgttt tggcgtggac ccagataaag aactttctta tcgccagcag |
| 121 | gcggccaatc tgcttcagga catggggcag cgtcttaacg tctcacaatt gactatcaac |
| 181 | actgctatag tatacatgca tcgattctac atgattcagt ccttcacaca gttccctgga |
| 241 | aattctgtgg ctccagcagc cttgtttcta gcagctaaag tggaggagca gcccaaaaaa |
| 301 | ttggaacatg tcatcaaggt agcacatact tgtctccatc ctcaggaatc ccttcctgat |
| 361 | actagaagtg aggcttattt gcaacaagtt caagatctgg tcattttaga aagcataatt |
| 421 | ttgcagactt taggctttga actaacaatt gatcacccac atactcatgt agtaaagtgc |
| 481 | actcaacttg ttcgagcaag caaggactta gcacagactt cttacttcat ggcaaccaac |
| 541 | agcctgcatt tgaccacatt tagcctgcag tacacacctc ctgtggtggc ctgtgtctgc |
| 601 | attcacctgg cttgcaagtg gtccaattgg gagatcccag tctcaactga cgggaagcac |
| 661 | tggtgggagt atgttgacgc cactgtgacc ttggaacttt tagatgaact gacacatgag |
| 721 | tttctacaga ttttggagaa aactcccaac aggctcaaac gcatttggaa ttggagggca |
| 781 | tgcgaggctg ccaagaaaac aaaagcagat gaccgaggaa cagatgaaaa gacttcagag |
| 841 | cagacaatcc tcaatatgat ttcccagagc tcttcagaca caaccattgc aggtttaatg |
| 901 | agcatgtcaa cttctaccac aagtgcagtg ccttccctgc cagtctccga agagtcatcc |
| 961 | agcaacttaa ccagtgtgga gatgttgccg ggcaagcgtt ggctgtcctc ccaaccttct |
| 1021 | ttcaaactag aacctactca gggtcatcgg actagtgaga atttagcact tacaggagtt |
| 1081 | gatcattcct taccacagga tggttcaaat gcatttattt cccagaagca gaatagtaag |
| 1141 | agtgtgccat cagctaaagt gtcactgaaa gaataccgcg cgaagcatgc agaagaattg |
| 1201 | gctgcccaga agaggcaact ggagaacatg gaagccaatg tgaagtcaca atatgcatat |
| 1261 | gctgcccaga atctcctttc tcatcatgat agccattctt cagtcattct aaaaatgccc |
| 1321 | atagagggtt cagaaaaccc cgagcggcct tttctggaaa aggctgacaa aacagctctc |
| 1381 | aaaatgagaa tcccagtggc aggtggagat aaagctgcgt cttcaaaacc agaggagata |
| 1441 | aaaatgcgca taaaagtcca tgctgcagct gataagcaca attctgtaga ggacagtgtt |
| 1501 | acaaagagcc gagagcacaa agaaaagcac aagactcacc catctaatca tcatcatcat |
| 1561 | cataatcacc actcacacaa gcactctcat tcccaacttc cagttggtac tgggaacaaa |
| 1621 | cgtcctggtg atccaaaaca tagtagccag acaagcaact tagcacataa aacctatagc |
| 1681 | ttgtctagtt ctttttcctc ttccagttct actcgtaaaa ggggaccctc tgaagagact |
| 1741 | ggaggggctg tgtttgatca tccagccaag attgccaaga gtactaaatc ctcttcccta |
| 1801 | aatttctcct tcccttcact tcctacaatg ggtcagatgc ctgggcatag ctcagacaca |
| 1861 | agtggccttt ccttttcaca gcccagctgt aaaactcgtg tccctcattc gaaactggat |
| 1921 | aaagggccca ctggggccaa tggtcacaac acgacccaga caatagacta tcaagacact |
| 1981 | gtgaatatgc ttcactccct gctcagtgcc cagggtgttc agcccactca gcccactgca |
| 2041 | tttgaatttg ttcgtcctta tagtgactat ctgaatcctc ggtctggtgg aatctcctcg |
| 2101 | agatctggca atacagacaa accccggcca ccacctctgc catcagaacc tcctccacca |
| 2161 | cttccacccc ttcctaagta a |

(SEQ ID NO:5; NCBI Reference No. NM_001240.3). An exemplary nucleotide sequence encoding human CCND3 is as follows:

| | |
|---|---|
| 1 | atgaactacc tggatcgcta cctgtcttgc gtccccaccc gaaaggcgca gttgcagctc |
| 61 | ctgggtgcgg tctgcatgct gctggcctcc aagctgcgcg agaccacgcc cctgaccatc |
| 121 | gaaaaactgt gcatctacac cgaccacgct gtctctcccc gccagttgcg ggactgggag |
| 181 | gtgctggtcc tagggaagct caagtgggac ctggctgctg tgattgcaca tgatttcctg |
| 241 | gccttcattc tgcaccggct ctctctgccc cgtgaccgac aggccttggt caaaaagcat |
| 301 | gcccagacct ttttggccct ctgtgctaca gattatacct ttgccatgta cccgccatcc |
| 361 | atgatcgcca cgggcagcat tggggctgca gtgcaaggcc tgggtgcctg ctccatgtcc |
| 421 | ggggatgagc tcacagagct gctggcaggg atcactggca ctgaagtgga ctgcctgcgg |
| 481 | gcctgtcagg agcagatcga agctgcactc agggagagcc tcagggaagc ctctcagacc |
| 541 | agctccagcc cagcgcccaa agccccccgg ggctccagca gccaagggcc cagccagacc |
| 601 | agcactccta cagatgtcac agccatacac ctgtag |

(SEQ ID NO:6; NCBI Reference No. NM_001136017.3).

An exemplary nucleotide sequence encoding human Cyclin L1 (CCNL1) is as follows:

| | |
|---|---|
| 1 | atggcgtccg ggcctcattc gacagctact gctgccgcag ccgcctcatc ggccgcccca |
| 61 | agcgcgggcg gctccagctc cgggacgacg accacgacga cgaccacgac gggagggatc |
| 121 | ctgatcggcg atcgcctgta ctcggaagtt tcacttacca tcgaccactc tctgattccg |
| 181 | gaggagaggc tctcgcccac cccatccatg caggatgggc tcgacctgcc cagtgagacg |
| 241 | gacttacgca tcctgggctg cgagctcatc caggccgccg gcattctcct ccggctgccg |
| 301 | caggtggcga tggcaacggg gcaggtgttg tttcatcgtt ttttctactc caaatctttc |
| 361 | gtcaaacaca gtttcgagat tgttgctatg gcttgtatta atcttgcatc aaaaatcgaa |
| 421 | gaagcaccta gaagaataag agatgtgatt aatgtattcc accacctccg ccagttaaga |
| 481 | ggaaaaagga ctccaagccc cctgatcctt gatcagaact acattaacac caaaaatcaa |
| 541 | gttatcaaag cagagaggag ggtgctaaag gagttgggat tttgtgttca tgtcaagcat |
| 601 | cctcataaga tcattgttat gtatttacaa gtcttagaat gtgaacgtaa tcaaaccctg |
| 661 | gttcaaactg cctggaatta catgaatgac agtcttcgaa ccaatgtgtt tgttcgattt |
| 721 | caaccagaga ctatagcatg tgcttgcatc taccttgcag ctagagcact tcagattccg |
| 781 | ttgccaactc gtccccattg gtttcttctt tttggtacta cagaagagga aatccaggaa |
| 841 | atctgcatag aaacacttag gctttatacc agaaaaaagc caaactatga attactggaa |
| 901 | aaagaagtag aaaaaagaaa agtagcctta caagaagcca aattaaaagc aaagggattg |
| 961 | aatccggatg gaactccagc cctttcaacc ctgggtggat tttctccagc ctccaagcca |
| 1021 | tcatcaccaa gagaagtaaa agctgaagag aaatcaccaa tctccattaa tgtgaagaca |
| 1081 | gtcaaaaaag aacctgagga tagacaacag gcttccaaaa gcccttacaa tggtgtaaga |
| 1141 | aaagacagca agagaagtag aaatagcaga agtgcaagtc gatcgaggtc aagaacacga |
| 1201 | tcacgttcta gatcacatac tccaagaaga cactataata ataggcggag tcgatctgga |
| 1261 | acatacagct cgagatcaag aagcaggtcc cgcagtcaca gtgaaagccc tcgaagacat |
| 1321 | cataatcatg gttctcctca ccttaaggcc aagcatacca gagatgattt aaaaagttca |
| 1381 | aacagacatg gtcataaaag gaaaaaatct cgttctcgat ctcagagcaa gtctcgggat |
| 1441 | cactcagatg cagccaagaa acacaggcat gaaaggggac atcataggga caggcgtgaa |
| 1501 | cgatctcgct cctttgagag gtcccataaa agcaagcacc atggtggcag tcgctcagga |
| 1561 | catggcaggc acaggcgctg a |

(SEQ ID NO:7; NCBI Reference No. NM_020307.2). An exemplary nucleotide sequence encoding human Cyclin L2 is as follows:

| | |
|---|---|
| 1 | atggcggcgg cggcggcggc ggctggtgct gcagggtcgg cagctcccgc ggcagcggcc |
| 61 | ggcgccccgg gatctggggg cgcaccctca gggtcgcagg gggtgctgat cggggacagg |
| 121 | ctgtactccg gggtgctcat caccttggag aactgcctcc tgcctgacga caagctccgt |
| 181 | ttcacgccgt ccatgtcgag cggcctcgac accgacacag agaccgacct ccgcgtggtg |
| 241 | ggctgcgagc tcatccaggc ggccggtatc ctgctccgcc tgccgcaggt ggccatggct |
| 301 | accgggcagg tgttgttcca gcggttcttt tataccaagt ccttcgtgaa gcactccatg |
| 361 | gagcatgtgt caatggcctg tgtccacctg gcttccaaga tagaagaggc cccaagacgc |
| 421 | atacgggacg tcatcaatgt gtttcaccgc cttcgacagc tgagagacaa aaagaagccc |
| 481 | gtgcctctac tactggatca agattatgtt aatttaaaga accaaattat aaaggcggaa |
| 541 | agacgagttc tcaaagagtt gggtttctgc gtccatgtga agcatcctca taagataatc |
| 601 | gttatgtacc ttcaggtgtt agagtgtgag cgtaaccaac acctggtcca gacctcatgg |
| 661 | aattacatga acgacagcct tcgcaccgac gtcttcgtgc ggttccagcc agagagcatc |
| 721 | gcctgtgcct gcatttatct tgctgcccgg acgctggaga tccctttgcc caatcgtccc |
| 781 | cattggtttc ttttgtttgg agcaactgaa gaagaaattc aggaaatctg cttaaagatc |
| 841 | ttgcagcttt atgctcggaa aaaggttgat ctcacacacc tggagggtga agtggaaaaa |
| 901 | agaaagcacg ctatcgaaga ggcaaaggcc caagcccggg gcctgttgcc tgggggcaca |
| 961 | caggtgctgg atggtacctc ggggttctct cctgccccca agctggtgga atcccccaaa |
| 1021 | gaaggtaaag ggagcaagcc ttccccactg tctgtgaaga acaccaagag gaggctggag |
| 1081 | ggcgccaaga aagccaaggc ggacagcccc gtgaacggct tgccaaaggg gcgagagagt |
| 1141 | cggagtcgga gccggagccg tgagcagagc tactcgaggt ccccatcccg atcagcgtct |
| 1201 | cctaagagga ggaaaagtga cagcggctcc acatctggtg ggtccaagtc gcagagccgc |
| 1261 | tcccggagca ggagtgactc cccaccgaga caggcccccc gcagcgctcc ctacaaaggc |
| 1321 | tctgagattc ggggctcccg gaagtccaag gactgcaagt acccccagaa gccacacaag |
| 1381 | tctcggagcc ggagttcttc ccgttctcga agcaggtcac gggagcgggc ggataatccg |
| 1441 | ggaaaataca agaagaaaag tcattactac agagatcagc gacgagagcg ctcgaggtcg |
| 1501 | tatgaacgca caggccgtcg ctatgagcgg gaccaccctg ggcacagcag gcatcggagg |
| 1561 | tga |

(SEQ ID NO:8; NCBI Reference No. NM_030937.4).

An exemplary nucleotide sequence encoding human IMPDH1 is as follows:

| | |
|---|---|
| 1 | atggaggggc cactcactcc accaccgctg cagggaggcg gagccgccgc tgttccggag |
| 61 | cccggagccc ggcaacaccc gggacacgag acggcggcgc agcggtacag cgcccgactg |
| 121 | ctgcaggccg gctacgagcc cgagagccct agattggacc tcgctacaca cccgacgaca |
| 181 | ccccgttcag aactatcttc agtggtctta ctggcaggtg ttggtgtcca gatggatcgc |
| 241 | cttcgcaggg ctagcatggc ggactacctg atcagcggcg gcaccggcta cgtgcccgag |
| 301 | gatgggctca ccgcgcagca gctcttcgcc agcgccgacg gcctcaccta caacgacttc |
| 361 | ctgattctcc caggattcat agacttcata gctgatgagg tggacctgac ctcagccctg |
| 421 | acccggaaga tcacgctgaa gacgccactg atctcctccc ccatggacac tgtgacagag |
| 481 | gctgacatgg ccattgccat ggctctgatg ggaggtattg gtttcattca ccacaactgc |
| 541 | accccagagt tccaggccaa cgaggtgcgg aaggtcaaga agtttgaaca gggcttcatc |
| 601 | acggaccctg tggtgctgag cccctcgcac actgtgggcg atgtgctgga ggccaagatg |
| 661 | cggcatggct tctctggcat ccccatcact gagacgggca ccatgggcag caagctggtg |
| 721 | ggcatcgtca cctcccgaga catcgacttt cttgctgaga aggaccacac caccctcctc |
| 781 | agtgaggtga tgacgccaag gattgaactg gtggtggctc cagcaggtgt gacgttgaaa |
| 841 | gaggcaaatg agatcctgca gcgtagcaag aaagggaagc tgcctatcgt caatgattgc |
| 901 | gatgagctgg tggccatcat cgcccgcacc gacctgaaga agaaccgaga ctaccctctg |
| 961 | gcctccaagg attcccagaa gcagctgctc tgtggggcag ctgtgggcac ccgtgaggat |
| 1021 | gacaaatacc gtctggacct gctcacccag gcgggcgtcg acgtcatagt cttggactcg |
| 1081 | tcccaaggga attcggtgta tcagatcgcc atggtgcatt acatcaaaca gaagtacccc |
| 1141 | cacctccagg tgattggggg gaacgtggtg acagcagccc aggccaagaa cctgattgat |
| 1201 | gctggtgtgg acgggctgcg cgtgggcatg ggctgcggct ccatctgcat cacccaggaa |
| 1261 | gtgatggcct gtggtcggcc ccagggcact gctgtgtaca aggtggctga gtatgcccgg |
| 1321 | cgctttggtg tgcccatcat agccgatggc ggcatccaga ccgtgggaca cgtggtcaag |
| 1381 | gccctggccc ttggagcctc cacagtgatg atgggctccc tgctggccgc cactacggag |
| 1441 | gcccctggcg agtacttctt ctcagacggg gtgcggctca agaagtaccg gggcatgggc |
| 1501 | tcactggatg ccatggagaa gagcagcagc agccagaaac gatacttcag cgagggggat |
| 1561 | aaagtgaaga tcgcgcaggg tgtctcgggc tccatccagg acaaaggatc cattcagaag |
| 1621 | ttcgtgccct acctcatagc aggcatccaa cacggctgcc aggatatcgg ggcccgcagc |
| 1681 | ctgtctgtcc ttcggtccat gatgtactca ggagagctca agtttgagaa gcggaccatg |
| 1741 | tcggcccaga ttgagggtgg tgtccatggc ctgcactctt acgaaaagcg gctgtactga |

(SEQ ID NO:19; NCBI Reference No. NM_000883.3). An exemplary nucleotide sequence encoding human IMPDH2 is as follows:

| | |
|---|---|
| 1 | atggccgact acctgattag tgggggcacg tcctacgtgc cagacgacgg actcacagca |
| 61 | cagcagctct tcaactgcgg agacggcctc acctacaatg actttctcat tctccctggg |
| 121 | tacatcgact tcactgcaga ccaggtggac ctgacttctg ctctgaccaa gaaaatcact |
| 181 | cttaagaccc cactggtttc ctctcccatg gacacagtca cagaggctgg gatggccata |
| 241 | gcaatggcgc ttacaggcgg tattggcttc atccaccaca actgtacacc tgaattccag |
| 301 | gccaatgaag ttcggaaagt gaagaaatat gaacagggat tcatcacaga ccctgtggtc |
| 361 | ctcagcccca aggatcgcgt gcgggatgtt tttgaggcca aggcccggca tggtttctgc |
| 421 | ggtatcccaa tcacagacac aggccggatg gggagccgct tggtgggcat catctcctcc |
| 481 | agggacattg attttctcaa agaggaggaa catgactgtt tcttggaaga gataatgaca |
| 541 | aagagggaag acttggtggt agcccctgca ggcatcacac tgaaggaggc aaatgaaatt |
| 601 | ctgcagcgca gcaagaaggg aaagttgccc attgtaaatg aagatgatga gcttgtggcc |
| 661 | atcattgccc ggacagacct gaagaagaat cgggactacc cactagcctc caaagatgcc |
| 721 | aagaaacagc tgctgtgtgg ggcagccatt ggcactcatg aggatgacaa gtataggctg |
| 781 | gacttgctcg cccaggctgg tgtggatgta gtggttttgg actcttccca gggaaattcc |
| 841 | atcttccaga tcaatatgat caagtacatc aaagacaaat accctaatct ccaagtcatt |
| 901 | ggaggcaatg tggtcactgc tgcccaggcc aagaacctca ttgatgcagg tgtggatgcc |
| 961 | ctgcgggtgg gcatgggaag tggctccatc tgcattacgc aggaagtgct ggcctgtggg |
| 1021 | cggccccaag caacagcagt gtacaaggtg tcagagtatg cacggcgctt tggtgttccg |
| 1081 | gtcattgctg atggaggaat ccaaaatgtg ggtcatattg cgaaagcctt ggcccttggg |
| 1141 | gcctccacag tcatgatggg ctctctcctg gctgccacca ctgaggcccc tggtgaatac |
| 1201 | ttcttttccg atgggatccg gctaaagaaa tatcgcggta tgggttctct cgatgccatg |
| 1261 | gacaagcacc tcagcagcca gaacagatat ttcagtgaag ctgacaaaat caaagtggcc |
| 1321 | cagggagtgt ctggtgctgt gcaggacaaa gggtcaatcc acaaatttgt cccttacctg |
| 1381 | attgctggca tccaacactc atgccaggac attggtgcca agagcttgac ccaagtccga |
| 1441 | gccatgatgt actctgggga gcttaagttt gagaagagaa cgtcctcagc ccaggtggaa |
| 1501 | ggtggcgtcc atagcctcca ttcgtatgag aagcggcttt tctga |

(SEQ ID NO:21; NCBI Reference No. NM_000884.2). It is understood that the sequences provided herein are provided as examples, but are in no way limiting. For example, nucleic acid inhibitors can be generated, and/or antibodies may be raised, e.g., to isoforms of any of the gene products described herein.

Antibodies

Although antibodies most often used to inhibit the activity extracellular proteins (e.g., receptors and/or ligands), the use of intracellular antibodies to inhibit protein function in a cell is also known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Lett. 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Biotechnology (NY) 12:396-399; Chen, S-Y. et al. (1994) Hum. Gene Ther. 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al., each of which is expressly incorporated by reference). Therefore, antibodies specific for any of the gene products described herein are useful as biological agents for the methods of the present invention.

Antibodies can be produced using a variety of known techniques, such as the standard somatic cell hybridization technique described by Kohler and Milstein, Nature 256: 495 (1975), which is expressly incorporated by reference. Additionally, other techniques for producing monoclonal antibodies known in the art can also be employed, e.g., viral or oncogenic transformation of B lymphocytes, phage display technique using libraries of human antibody genes.

Polyclonal antibodies can be prepared by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well known techniques, such as protein A chromatography to obtain the IgG fraction. At an appropriate time after immunization, e.g., when the antibody titers are highest, antibody-producing cells can be obtained from the subject and used to prepare monoclonal antibodies.

Any of the many well-known protocols used for fusing lymphocytes and immortalized cell lines can be applied for the purpose of generating monoclonal antibodies specific against TDP-43 (see, e.g., Galfre, G. et al. (1977) Nature 266:55052; Gefter et al. (1977) supra; Lerner (1981) supra; Kenneth (1980) supra), each of which is expressly incorporated by reference. Moreover, the ordinary skilled worker will appreciate that there are many variations of such methods which also would be useful. Typically, an immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. An example of an appropriate mouse cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a given polypeptide, e.g., using a standard ELISA assay.

Monoclonal antibodies can be identified and isolated by screening a recombinant combinatorial immunoglobulin library (e.g., an antibody phage or yeast display library) with the appropriate gene product (e.g., CCNT1) or antigenic fragment thereof to thereby isolate immunoglobulin library members that bind to the gene product. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612), and methods for screening phage and yeast display libraries are known in the art. Examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554, each of which is expressly incorporated by reference.

In addition, chimeric and humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743, each of which is expressly incorporated by reference.

In another embodiment, human monoclonal antibodies can be generated using transgenic or transchromosomal mice carrying parts of the human immune system rather than the mouse system. In one embodiment, transgenic mice, referred to herein as “humanized mice,” which contain a human immunoglobulin gene miniloci that encodes unrearranged human heavy and light chain variable region immunoglobulin sequences, together with targeted mutations that inactivate or delete the endogenous μ and κ chain loci (Lonberg, N. et al. (1994) Nature 368(6474): 856 859, which is expressly incorporated by reference). The mice may also contain human heavy chain constant region immunoglobulin sequences. Accordingly, the mice express little or no mouse IgM or κ, and in response to immunization, the introduced human heavy and light chain variable region transgenes undergo class switching and somatic mutation to generate high affinity human variable region antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N. Y Acad. Sci 764:536 546, each of which is expressly incorporated by reference). These mice can be used to generate fully human monoclonal antibodies using the techniques described above or any other technique known in the art. The preparation of humanized mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci USA 90:3720 3724; Choi et al. (1993) Nature Genetics 4:117 123; Chen, J. et al. (1993) EMBO J. 12: 821 830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay, and GenPharm International; U.S. Pat. No. 5,545,807 to Surani et al., each of which is expressly incorporated by reference.

Exemplary amino acid sequences for the gene products described herein, from multiple species including human, are known in the art and are publicly available. For example, an exemplary amino acid sequence for human RNGTT (isoform A) is as follows:

| | |
|---|---|
| 1 | mahnkipprw lncprrgqpv agrflplktm lgprydsqva eenrfhpsml snylkslkvk |
| 61 | mgllvdltnt srfydrndie kegikyiklq ckghgecptt entetfirlc erfnernppe |
| 121 | ligvhcthgf nrtgflicaf lvekmdwsie aavatfaqar ppgiykgdyl kelfrrygdi |
| 181 | eeappppllp dwcfeddede dededgkkes epgssasfgk rrkerlklga iflegvtvkg |
| 241 | vtqvttqpkl gevqqkchqf cgwegsgfpg aqpvsmdkqn iklldlkpyk vswkadgtry |
| 301 | mmlidgtnev fmidrdnsvf hvsnlefpfr kdlrmhlsnt lldgemiidr vngqavpryl |
| 361 | iydiikfnsq pvgdcdfnvr lqciereiis prhekmktgl idktqepfsv rnkpffdict |
| 421 | srkllegnfa kevshemdgl ifqptgkykp grcddilkwk ppslnsvdfr lkitrmggeg |
| 481 | llpqnvglly vggyerpfaq ikvtkelkqy dnkiieckfe nnswvfmrqr tdksfpnayn |
| 541 | tamavcnsis npvtkemlfe fidrctaasq gqkrkhhldp dtelmppppp krprplt |

(SEQ ID NO:9; NCBI Reference No. NP_003791.3). An exemplary amino acid sequence for human RNMT is set forth below:

| | |
|---|---|
| 1 | mansakaeey ekmsleqaka svnsetessf ninenttasg tglsektsvc rqvdiarkrk |
| 61 | efeddlvkes sscgkdtpsk krkldpeivp eekdcgdaeg nskkrkrete dvpkdksstg |
| 121 | dgtqnkrkia ledvpekqkn leeghsstva ahynelqevg lekrsqsrif ylrnfnnwmk |
| 181 | svligeflek vrqkkkrdit vldlgcgkgg dllkwkkgri nklvctdiad vsvkqcqqry |
| 241 | edmknrrdse yifsaefita dsskellidk frdpqmcfdi cscqfvchys fesyeqadmm |
| 301 | lrnacerlsp ggyfigttpn sfelirrlea setesfgnei ytvkfqkkgd yplfgckydf |
| 361 | nlegvvdvpe flvyfpllne makkynmklv ykktflefye ekiknnenkm llkrmqalep |
| 421 | ypanessklv sekvddyeha akymknsqvr lplgtlskse weatsiylvf afekqq |

(SEQ ID NO:10; NCBI Reference No. NP_003790.1).

An exemplary amino acid sequence for human Cdk11(A) is as follows:

| | |
|---|---|
| 1 | mgdekdswkv ktldeilqek krrkeqeeka eikrlknsdd rdskrdslee gelrdhcmei |
| 61 | tirnspyrre dsmedrgeed dslaikppqq msrkekvhhr kdekrkekcr hhshsaeggk |
| 121 | harvkerehe rrkrhreeqd karrewerqk rremarehsr rerdrleqle rkrererkmr |
| 181 | eqqkeqreqk ererraeerr kerearrevs ahhrtmredy sdkvkashws rspprpprer |
| 241 | felgdgrkpv keekmeerdl lsdlqdisds erktssaess saesgsgsee eeeeeeeeee |
| 301 | egstseesee eeeeeeeeee etgsnseeas eqsaeevsee emsedeeren enhllvvpes |
| 361 | rfdrdsgese eaeeevgegt pqssaltegd yvpdspallp ielkqelpky lpalqgcrsv |
| 421 | eefqclnrie egtygvvyra kdkktdeiva lkrlkmekek egfpitslre intilkaqhp |
| 481 | nivtvreivv gsnmdkiyiv mnyvehdlks lmetmkqpfl pgevktlmiq llrgvkhlhd |
| 541 | nwilhrdlkt snlllshagi lkvgdfglar eygsplkayt pvvvtqwyra pelllgakey |
| 601 | stavdmwsvg cifgelltqk plfpgnseid qinkvfkelg tpsekiwpgy selpvvkkmt |
| 661 | fsehpynnlr krfgallsdq gfdlmnkflt yfpgrrisae dglkheyfre tplpidpsmf |
| 721 | ptwpakseqq rvkrgtsprp pegglgysql gdddlketgf hltttnqgas aagpgfslkf |

(SEQ ID NO:11; NCBI Reference No. NP_076916.2).

An exemplary amino acid sequence for human Cdk11B is as follows:

| | |
|---|---|
| 1 | mgdekdswkv ktldeilqek krrkeqeeka eikrlknsdd rdskrdslee gelrdhrmei |
| 61 | tirnspyrre dsmedrgeed dslaikppqq msrkekahhr kdekrkekrr hrshsaeggk |
| 121 | harvkekere herrkrhree qdkarrewer qkrremareh srrerdrleq lerkrererk |
| 181 | mreqqkeqre qkererraee rrkerearre vsahhrtmre dysdkvkash wsrspprppr |
| 241 | erfelgdgrk pgearpapaq kpaqlkeekm eerdllsdlq disdserkts saesssaesg |
| 301 | sgseeeeeee eeeeeegsts eeseeeeeee eeeeeetgsn seeaseqsae evseeemsed |
| 361 | eerenenhll vvpesrfdrd sgeseeaeee vgegtpqssa ltegdyvpds palspielkq |
| 421 | elpkylpalq gcrsveefqc lnrieegtyg vvyrakdkkt deivalkrlk mekekegfpi |
| 481 | tslreintil kaqhpnivtv reivvgsnmd kiyivmnyve hdlkslmetm kqpflpgevk |
| 541 | tlmiqllrgv khlhdnwilh rdlktsnlll shagilkvgd fglareygsp lkaytpvvvt |
| 601 | lwyrapelll gakeystavd mwsvgcifge lltqkplfpg kseidqinkv fkdlgtpsek |
| 661 | iwpgyselpa vkkmtfsehp ynnlrkrfga llsdqgfdlm nkfltyfpgr risaedglkh |
| 721 | eyfretplpi dpsmfptwpa kseqqrvkrg tsprppeggl gysqlgdddl ketgfhlttt |
| 781 | nqgasaagpg fslkf |

(SEQ ID NO:13; NCBI Reference No. NP_001778.2). An exemplary amino acid sequence for human Cdk9 is as follows:

| | |
|---|---|
| 1 | makqydsvec pfcdevskye klakigqgtf gevfkarhrk tgqkvalkkv lmenekegfp |
| 61 | italreikil qllkhenvvn lieicrtkas pynrckgsiy lvfdfcehdl agllsnvlvk |
| 121 | ftlseikrvm qmllnglyyi hrnkilhrdm kaanvlitrd gvlkladfgl arafslakns |
| 181 | qpnrytnrvv tlwyrppell lgerdygppi dlwgagcima emwtrspimq gnteqhqlal |
| 241 | isqlcgsitp evwpnvdnye lyeklelvkg qkrkvkdrlk ayvrdpyald lidkllvldp |
| 301 | aqridsddal nhdffwsdpm psdlkgmlst hltsmfeyla pprrkgsqit qqstnqsrnp |
| 361 | attnqtefer vf |

(SEQ ID NO:14; NCBI Reference No. NP_001252.1). An exemplary amino acid sequence for human CCNT1 is as follows:

| | |
|---|---|
| 1 | megerknnnk rwyftreqle nspsrrfgvd pdkelsyrqq aanllqdmgq rlnvsqltin |
| 61 | taivymhrfy miqsftqfpg nsvapaalfl aakveeqpkk lehvikvaht clhpqeslpd |
| 121 | trseaylqqv qdlvilesii lqtlgfelti dhphthvvkc tqlvraskdl aqtsyfmatn |
| 181 | slhlttfslq ytppvvacvc ihlackwsnw eipvstdgkh wweyvdatvt lelldelthe |
| 241 | flqilektpn rlkriwnwra ceaakktkad drgtdektse qtilnmisqs ssdttiaglm |
| 301 | smststtsav pslpvseess snltsvemlp gkrwlssqps fkleptqghr tsenlaltgv |
| 361 | dhslpqdgsn afisqkqnsk svpsakvslk eyrakhaeel aaqkrqlenm eanvksqyay |
| 421 | aaqnllshhd shssvilkmp iegsenperp flekadktal kmripvaggd kaasskpeei |
| 481 | kmrikvhaaa dkhnsvedsv tksrehkekh kthpsnhhhh hnhhshkhsh sqlpvgtgnk |
| 541 | rpgdpkhssq tsnlahktys lsssfsssss trkrgpseet ggavfdhpak iakstksssl |
| 601 | nfsfpslptm gqmpghssdt sglsfsqpsc ktrvphskld kgptganghn ttqtidyqdt |
| 661 | vnmlhsllsa qgvqptqpta fefvrpysdy lnprsggiss rsgntdkprp pplpsepppp |
| 721 | lpplpk |

(SEQ ID NO:15; NCBI Reference No. NP_001231.2).

An exemplary amino acid sequence for human Cyclin L1 is as follows:

| | |
|---|---|
| 1 | masgphstat aaaaassaap saggsssgtt tttttttggi ligdrlysev sltidhslip |
| 61 | eerlsptpsm qdgldlpset dlrilgceli qaagillrlp qvamatgqvl fhrffysksf |
| 121 | vkhsfeivam acinlaskie eaprrirdvi nvfhhlrqlr gkrtpsplil dqnyintknq |
| 181 | vikaerrvlk elgfcvhvkh phkiivmylq vlecernqtl vqtawnymnd slrtnvfvrf |
| 241 | qpetiacaci ylaaralqip lptrphwfll fgtteeeiqe icietlrlyt rkkpnyelle |
| 301 | kevekrkval qeaklkakgl npdgtpalst lggfspaskp ssprevkaee kspisinvkt |
| 361 | vkkepedrqq askspyngvr kdskrsrnsr sasrsrsrtr srsrshtprr hynnrrsrsg |
| 421 | tyssrsrsrs rshsesprrh hnhgsphlka khtrddlkss nrhghkrkks rsrsqsksrd |
| 481 | hsdaakkhrh erghhrdrre rsrsfershk skhhggsrsg hgrhrr |

(SEQ ID NO:16; NCBI Reference No. NP_064703.1). An exemplary amino acid sequence for human Cyclin L2 is as follows:

| | |
|---|---|
| 1 | maaaaaaaga agsaapaaaa gapgsggaps gsqgvligdr lysgvlitle ncllpddklr |
| 61 | ftpsmssgld tdtetdlrvv gceliqaagi llrlpqvama tgqvlfqrff ytksfvkhsm |
| 121 | ehvsmacvhl askieeaprr irdvinvfhr lrqlrdkkkp vpllldqdyv nlknqiikae |
| 181 | rrvlkelgfc vhvkhphkii vmylqvlece rnqhlvqtsw nymndslrtd vfvrfqpesi |
| 241 | acaciylaar tleiplpnrp hwfllfgate eeiqeiclki lqlyarkkvd lthlegevek |
| 301 | rkhaieeaka qargllpggt qvldgtsgfs papklvespk egkgskpspl svkntkrrle |
| 361 | gakkakadsp vnglpkgres rsrsrsreqs ysrspsrsas pkrrksdsgs tsggsksqsr |
| 421 | srsrsdsppr qaprsapykg seirgsrksk dckypqkphk srsrsssrsr srsreradnp |
| 481 | gkykkkshyy rdqrrersrs yertgrryer dhpghsrhrr |

(SEQ ID NO:17; NCBI Reference No. NP_112199.2). An exemplary amino acid sequence for human CCND3 is as follows:

| | |
|---|---|
| 1 | mnyldrylsc vptrkaqlql lgavcmllas klrettplti eklciytdha vsprqlrdwe |
| 61 | vlvlgklkwd laaviahdfl afilhrlslp rdrqalvkkh aqtflalcat dytfamypps |
| 121 | miatgsigaa vqglgacsms gdeltellag itgtevdclr acqeqieaal reslreasqt |
| 181 | ssspapkapr gsssqgpsqt stptdvtaih l |

(SEQ ID NO:18; NCBI Reference No. NP_001129489.1). An exemplary amino acid sequence for human IMPDH1 is as follows:

| | |
|---|---|
| 1 | megpltpppl qgggaaavpe pgarqhpghe taaqrysarl lqagyepesp rldlathptt |
| 61 | prselssvvl lagvgvqmdr lrrasmadyl isggtgyvpe dgltaqqlfa sadgltyndf |
| 121 | lilpgfidfi adevdltsal trkitlktpl isspmdtvte admaiamalm ggigfihhnc |
| 181 | tpefqanevr kvkkfeqgfi tdpvvlspsh tvgdvleakm rhgfsgipit etgtmgsklv |
| 241 | givtsrdidf laekdhttll sevmtpriel vvapagvtlk eaneilqrsk kgklpivndc |
| 301 | delvaiiart dlkknrdypl askdsqkqll cgaavgtred dkyrldlltq agvdvivlds |
| 361 | sqgnsvyqia mvhyikqkyp hlqviggnvv taaqaknlid agvdglrvgm gcgsicitqe |
| 421 | vmacgrpqgt avykvaeyar rfgvpiiadg giqtvghvvk alalgastvm mgsllaatte |
| 481 | apgeyffsdg vrlkkyrgmg sldameksss sqkryfsegd kvkiaqgvsg siqdkgsiqk |
| 541 | fvpyliagiq hgcqdigars lsvlrsmmys gelkfekrtm saqieggvhg lhsyekrly |

(SEQ ID NO:20; NCBI Reference No. NP_000874.2). An exemplary amino acid sequence for human IMPDH2 is as follows:

| | |
|---|---|
| 1 | madylisggt syvpddglta qqlfncgdgl tyndflilpg yidftadqvd ltsaltkkit |
| 61 | lktplvsspm dtvteagmai amaltggigf ihhnctpefq anevrkvkky eqgfitdpvv |
| 121 | lspkdrvrdv feakarhgfc gipitdtgrm gsrlvgiiss rdidflkeee hdcfleeimt |
| 181 | kredlvvapa gitlkeanei lqrskkgklp ivneddelva iiartdlkkn rdyplaskda |
| 241 | kkqllcgaai gtheddkyrl dllaqagvdv vvldssqgns ifqinmikyi kdkypnlqvi |
| 301 | ggnvvtaaqa knlidagvda lrvgmgsgsi citqevlacg rpqatavykv seyarrfgvp |
| 361 | viadggiqnv ghiakalalg astvmmgsll aatteapgey ffsdgirlkk yrgmgsldam |
| 421 | dkhlssqnry fseadkikva qgvsgavqdk gsihkfvpyl iagiqhscqd igaksltqvr |
| 481 | ammysgelkf ekrtssaqve ggvhslhsye krlf |

(SEQ ID NO:22; NCBI Reference No. NP_000875.2).

Exemplary Compounds

Non-limiting, exemplary compounds for use in the methods and compositions described herein include, e.g., small molecule inhibitors of Cdk11 kinase activity, e.g., as described in U.S. Pat. Nos. 8,598,344 and 8,309,550 and International Patent Application Publication Nos. WO 2003/099811 and WO 2003/099796. siRNA inhibitors of Cdk11 are described in, e.g., U.S. Pat. No. 7,745,610 and Duan et al. (2012) Clin Cancer Res 18(17):4580-4588. Furthermore, screening methods useful for identifying additional inhibitors of Cdk11 are described in, e.g., Duan et al. (supra) and U.S. Patent Application Publication No. 20080050734.

Exemplary small molecule inhibitors of Cdk9 kinase activity are described in, e.g., Walsby et al. (2014) Oncotarget 5(2):375-385; International Patent Application Publication Nos. WO 2013/026874, WO 2013/059634, WO 2014/160028, and WO 2012/101065, European Patent Publication Nos. EP2562265 and EP 2668162; and U.S. Patent Application Publication No. 20140275153. Nucleic acid inhibitors of Cdk9 are known in the art and described in, e.g., David et al. (2009) EMBO Rep 11(11):876-882; Garriga and Grana (2014) BMC Research Notes 7:301; and U.S. Patent Application Publication No. 20040204377.

Inhibitors of RNGTT guanylyltransferase activity include, e.g., mizoribine (Picard-Jean et al. (2013) PLoS ONE 8(1):e54621), mycophenolic acid/mycophenolate sodium (Tremblay-Letourneau et al. (2011) PLoS ONE 6(9):e24806), mycophenolate mofetil (Kalluri et al. (2012) World J Transplant 2(4):51-68), and ribavirin (Bougie and Bisaillon (2004) J Biol Chem 279(21):22124-30). Examplary siRNA molecules useful for inhibiting expression of RNGTT are known in the art and available from Santa Cruz Biotechnology, Inc. (product no. sc-95119) and Origene (Rockville, Md.; product no. TR706470).

Likewise, exemplary inhibitors of IMPDH are known in the art. For example, small molecule inhibitors of IMPDH include, but are not limited to, mizoribine, mycophenolic acid/mycophenolate sodium, mycophenolate mofetil, azathioprine, and ribavirin (Kalluri et al. (2012) World J Transplant 2(4):51-68) and those described in U.S. Patent Application Publication Nos. 20020052513 and 20120220619, International Patent Application Publication Nos. WO 2003/035066 and WO 1997040028, and European Patent No. EP 1575579 B 1. Nucleic acid inhibitors of IMPDH are described in, e.g., International Patent Application Publication No. WO 2013/077228 and Domhan et al. (2008) Mol Cancer Ther 7:1656.

### Pharmaceutical Compositions and Formulations

The compositions described herein can be formulated as a pharmaceutical solution, e.g., for administration to a subject for treating a proliferative disorder. The pharmaceutical compositions will generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see e.g., Berge et al. (1977) J Pharm Sci 66:1-19).

The compositions can be formulated according to standard methods. Pharmaceutical formulation is a well-established art, and is further described in, e.g., Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X). In some embodiments, a composition can be formulated, for example, as a buffered solution at a suitable concentration and suitable for storage at 2-8° C. (e.g., 4° C.). In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. For example, compositions containing a composition intended for systemic or local delivery can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion (see below).

The compositions can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filtered sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

The compositions described herein can also be formulated in immunoliposome compositions. Such formulations can be prepared by methods known in the art such as, e.g., the methods described in Epstein et al. (1985) Proc Natl Acad Sci USA 82:3688; Hwang et al. (1980) Proc Natl Acad Sci USA 77:4030; and U.S. Pat. Nos. 4,485,045 and 4,544,545. Liposomes with enhanced circulation time are disclosed in, e.g., U.S. Pat. No. 5,013,556.

In certain embodiments, compositions can be formulated with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

In some embodiments, compositions described herein are administered in an aqueous solution by parenteral injection. The disclosure features pharmaceutical compositions comprising an effective amount of the agent (or more than one agent, and optionally include pharmaceutically acceptable diluents, preservatives, solubilizers, emulsifiers, adjuvants and/or carriers. Such compositions include sterile water, buffered saline (e.g., Tris-HCl, acetate, phosphate), pH and ionic strength; and optionally, additives such as detergents and solubilizing agents (e.g., TWEEN® 20, TWEEN 80, Polysorbate 80), anti-oxidants (e.g., ascorbic acid, sodium metabisulfite), and preservatives (e.g., thimersol, benzyl alcohol) and bulking substances (e.g., lactose, mannitol). The formulations may be sterilized, e.g., using filtration, incorporating sterilizing agents into the compositions, by irradiating the compositions, or by heating the compositions.

As described above, relatively high concentration compositions can be made. For example, the compositions can be formulated at a concentration of between about 10 mg/mL to 100 mg/mL (e.g., between about 9 mg/mL and 90 mg/mL; between about 9 mg/mL and 50 mg/mL; between about 10 mg/mL and 50 mg/mL; between about 15 mg/mL and 50 mg/mL; between about 15 mg/mL and 110 mg/mL; between about 15 mg/mL and 100 mg/mL; between about 20 mg/mL and 100 mg/mL; between about 20 mg/mL and 80 mg/mL; between about 25 mg/mL and 100 mg/mL; between about 25 mg/mL and 85 mg/mL; between about 20 mg/mL and 50 mg/mL; between about 25 mg/mL and 50 mg/mL; between about 30 mg/mL and 100 mg/mL; between about 30 mg/mL and 50 mg/mL; between about 40 mg/mL and 100 mg/mL; between about 50 mg/mL and 100 mg/mL; or between about 20 mg/mL and 50 mg/mL). In some embodiments, compositions can be formulated at a concentration of greater than 5 mg/mL and less than 50 mg/mL. Methods for formulating a protein in an aqueous solution are known in the art and are described in, e.g., U.S. Pat. No. 7,390,786; McNally and Hastedt (2007), “Protein Formulation and Delivery,” Second Edition, Drugs and the Pharmaceutical Sciences, Volume 175, CRC Press; and Banga (1995), “Therapeutic peptides and proteins: formulation, processing, and delivery systems,” CRC Press. In some embodiments, the aqueous solution has a neutral pH, e.g., a pH between, e.g., 6.5 and 8 (e.g., between and inclusive of 7 and 8). In some embodiments, the aqueous solution has a pH of about 6.6, 6.7, 6.8, 6.9, 7, 7.1, 7.2, 7.3, 7.4, 7.5, 7.6, 7.7, 7.8, 7.9, or 8.0. In some embodiments, the aqueous solution has a pH of greater than (or equal to) 6 (e.g., greater than or equal to 6.1, 6.2, 6.3, 6.4, 6.5, 6.6, 6.7, 6.8, 6.9, 7, 7.1, 7.2, 7.3, 7.4, 7.5, 7.6, 7.7, 7.8, or 7.9), but less than pH 8.

As used herein, “about” and like grammatical terms refers to an acceptable degree of error for the quantity measured given the nature or precision of the measurements. Exemplary degrees of error include up to 20% (e.g., no more than 19, 18, 17, 16, 15, 14, 12, 11, 10, 9, 8, 7, 6, 5, 4, 3, 2, 1, or less than 1%). In some embodiments, e.g., in biological systems, about includes values that are within an order of magnitude, e.g., within 4-fold, 3-fold, or 2-fold. In some embodiments, “about” refers to a value no more than 100% of the stated reference value.

Nucleic acids encoding a therapeutic polypeptide (e.g., a therapeutic antibody or a peptide inhibitor of one of the gene products described herein) can be incorporated into a gene construct to be used as a part of a gene therapy protocol to deliver nucleic acids that can be used to express and produce agents within cells. Expression constructs of such components may be administered in any therapeutically effective carrier, e.g. any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1 (HSV-1), or recombinant bacterial or eukaryotic plasmids. Viral vectors can transfect cells directly; plasmid DNA can be delivered with the help of, for example, cationic liposomes (lipofectin) or derivatized, polylysine conjugates, gramicidin S, artificial viral envelopes or other such intracellular carriers, as well as direct injection of the gene construct or CaPO₄ precipitation (see, e.g., WO04/060407) carried out in vivo. Examples of suitable retroviruses include pLJ, pZIP, pWE and pEM which are known to those skilled in the art (see, e.g., Eglitis et al. (1985) Science 230:1395-1398; Danos and Mulligan (1988) Proc Natl Acad Sci USA 85:6460-6464; Wilson et al. (1988) Proc Natl Acad Sci USA 85:3014-3018; Armentano et al. (1990) Proc. Natl. Acad. Sci. USA 87:6141-6145; Huber et al. (1991) Proc Natl Acad Sci USA 88:8039-8043; Ferry et al. (1991) Proc Natl Acad Sci USA 88:8377-8381; Chowdhury et al. (1991) Science 254:1802-1805; van Beusechem et al. (1992) Proc Natl Acad Sci USA 89:7640-7644; Kay et al. (1992) Human Gene Therapy 3:641-647; Dai et al. (1992) Proc Natl Acad Sci USA 89:10892-10895; Hwu et al. (1993) J Immunol 150:4104-4115; U.S. Pat. Nos. 4,868,116 and 4,980,286; PCT Publication Nos. WO89/07136, WO89/02468, WO89/05345, and WO92/07573). Another viral gene delivery system utilizes adenovirus-derived vectors (see, e.g., Berkner et al. (1988) BioTechniques 6:616; Rosenfeld et al. (1991) Science 252:431-434; and Rosenfeld et al. (1992) Cell 68:143-155). Suitable adenoviral vectors derived from the adenovirus strain Ad type 5 d1324 or other strains of adenovirus (e.g., Ad2, Ad3, Ad7, etc.) are known to those skilled in the art. Yet another viral vector system useful for delivery of the subject gene is the adeno-associated virus (AAV). See, e.g., Flotte et al. (1992) Am J Respir Cell Mot Biol 7:349-356; Samulski et al. (1989) J Virol 63:3822-3828; and McLaughlin et al. (1989) J Virol 62:1963-1973.

When compositions are to be used in combination with a second active agent, the compositions can be coformulated with the second agent or the compositions can be formulated separately from the second agent formulation. For example, the respective pharmaceutical compositions can be mixed, e.g., just prior to administration, and administered together or can be administered separately, e.g., at the same or different times (see below).

### Applications

The compounds described herein can be used in a number of in vitro, ex vivo, and in vivo applications. For example, the compounds described herein can be contacted to cultured cells in vitro or in vivo, or administered to a subject (e.g., a mammal, such as a human) to modulate the growth, activity, proliferation, metabolism, motility/mobility, or viability of a proliferating cell. For example, cultured cancer cells (e.g., cancer cell lines, such as those with increased mTORC1 activity and/or those bearing a mutation in one or both of the TSC1 and TSC2 genes) can be contacted with one or more of the compounds described herein in an amount effective to inhibit the proliferation of the cells.

In some embodiments, the methods described herein can involve detecting or measuring the expression of mTOR (e.g., overexpression) and/or mTOR complex 1 (mTORC1) activity (e.g., increased activity). Gene expression can be detected as, e.g., protein or mRNA expression of a target protein. That is, the presence or expression level (amount) of a protein can be determined by detecting and/or measuring the level of mRNA or protein expression of the protein.

mTOR (mammalian target of rapamycin) is a major regulator of cell growth and proliferation in response to both growth factors and cellular nutrients. It is a key regulator of the rate limiting step for translation of mRNA into protein, the binding of the ribosome to mRNA. mTOR exists in at least 2 distinct multiprotein complexes described as raptor-mTOR complex (mTORC1) and rictor-mTOR complex (mTORC2) in mammalian cells (sometimes referred to as just TORC1 and TORC2). The term “mTOR1” or “mTOR Complex 1 (mTORC1),” as used herein, means a complex composed of mTOR, regulatory-associated protein of mTOR (Raptor), mammalian LST8/G-protein (3-subunit like protein (mLST8/GβL), and, optionally, the recently identified partners PRAS40 and DEPTOR. mTORC1 is a rapamycin-sensitive complex as its kinase activity is inhibited by FKB12-rapamycin in vitro. The drug rapamycin does not displace GβL or raptor from mTOR but does strongly destabilize the raptor-mTOR interaction. Extensive work with rapamycin indicates that mTORC1 complex positively regulates cell growth. The raptor branch of the mTOR pathway modulates number of processes, including mRNA translation, ribosome biogenesis, nutrient metabolism and autophagy. The two mammalian proteins, S6 Kinase 1 (S6K1) and 4E-BP1, which are linked to protein synthesis, are downstream targets of mTORC1. mTORC1 has been shown to phosphorylates S6K1 at T389 and is inhibited by FKBP12-rapamycin in vitro and by rapamycin in vivo. mTORC1 can also phosphorylate 4E-BP1 at T37/46 in vitro and in vivo.

A variety of suitable methods can be employed to detect and/or measure the level of mRNA expression of a protein. For example, mRNA expression can be determined using

Northern blot or dot blot analysis, reverse transcriptase-PCR (RT-PCR; e.g., quantitative RT-PCR), in situ hybridization (e.g., quantitative in situ hybridization) or nucleic acid array (e.g., oligonucleotide arrays or gene chips) analysis. Details of such methods are described below and in, e.g., Sambrook et al., Molecular Cloning: A Laboratory Manual Second Edition vol. 1, 2 and 3. Cold Spring Harbor Laboratory Press: Cold Spring Harbor, N.Y., USA, November 1989; Gibson et al. (1999) Genome Res 6(10):995-1001; and Zhang et al. (2005) Environ Sci Technol 39(8):2777-2785; U.S. Patent Application Publication No. 2004086915; European Patent No. 0543942; and U.S. Pat. No. 7,101,663; the disclosures of each of which are incorporated herein by reference in their entirety.

In one example, the presence or amount of one or more discrete mRNA populations in a biological sample can be determined by isolating total mRNA from the biological sample (see, e.g., Sambrook et al. (supra) and U.S. Pat. No. 6,812,341) and subjecting the isolated mRNA to agarose gel electrophoresis to separate the mRNA by size. The size-separated mRNAs are then transferred (e.g., by diffusion) to a solid support such as a nitrocellulose membrane. The presence or amount of one or more mRNA populations in the biological sample can then be determined using one or more detectably-labeled polynucleotide probes, complementary to the mRNA sequence of interest, which bind to and thus render detectable their corresponding mRNA populations. Detectable labels include, e.g., fluorescent (e.g., fluorescein, fluorescein isothiocyanate, rhodamine, dichlorotriazinylamine fluorescein, dansyl chloride, allophycocyanin (APC), or phycoerythrin), luminescent (e.g., europium, terbium, Qdot™ nanoparticles supplied by the Quantum Dot Corporation, Palo Alto, Calif.), radiological (e.g., ¹²⁵I, ¹³¹I, ³⁵S, ³²P, ³³P, or ³H), and enzymatic (horseradish peroxidase, alkaline phosphatase, beta-galactosidase, or acetylcholinesterase) labels.

In another example, the presence or amount of discrete populations of mRNA in a biological sample can be determined using nucleic acid (or oligonucleotide) arrays (e.g., an array described below under “Arrays and Kits”). For example, isolated mRNA from a biological sample can be amplified using RT-PCR with random hexamer or oligo(dT)-primer mediated first strand synthesis. The RT-PCR step can be used to detectably-label the amplicons, or, optionally, the amplicons can be detectably labeled subsequent to the RT-PCR step. For example, the detectable label can be enzymatically (e.g., by nick translation or a kinase such as T4 polynucleotide kinase) or chemically conjugated to the amplicons using any of a variety of suitable techniques (see, e.g., Sambrook et al., supra). The detectably-labeled amplicons are then contacted to a plurality of polynucleotide probe sets, each set containing one or more of a polynucleotide (e.g., an oligonucleotide) probe specific for (and capable of binding to) a corresponding amplicon, and where the plurality contains many probe sets each corresponding to a different amplicon. Generally, the probe sets are bound to a solid support and the position of each probe set is predetermined on the solid support. The binding of a detectably-labeled amplicon to a corresponding probe of a probe set indicates the presence or amount of a target mRNA in the biological sample. Additional methods for detecting mRNA expression using nucleic acid arrays are described in, e.g., U.S. Pat. Nos. 5,445,934; 6,027,880; 6,057,100; 6,156,501; 6,261,776; and 6,576,424; the disclosures of each of which are incorporated herein by reference in their entirety.

Methods of detecting and/or for quantifying a detectable label depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

RNA can be extracted from the tissue sample by a variety of methods, e.g., the guanidium thiocyanate lysis followed by CsCl centrifugation (Chirgwin et al. 1979, Biochemistry 18:5294-5299). RNA from single cells can be obtained as described in methods for preparing cDNA libraries from single cells, such as those described in Dulac (1998) Curr Top Dev Biol 36:245 and Jena et al. (1996) J Immunol Methods 190:199. Care to avoid RNA degradation must be taken, e.g., by inclusion of RNAsin.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc Natl Acad Sci USA 86:9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by Marshall et al., (1994) PCR Methods and Applications 4: 80-84. Real time PCR may also be used.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace (1989) Genomics 4: 560; Landegren et al. (1988) Science 241:1077); self-sustained sequence replication (SSR) (see, e.g., Guatelli et al. (1990) Proc Nat Acad Sci USA 87:1874); and transcription amplification (see, e.g., Kwoh et al. (1989) Proc Natl Acad Sci USA 86:1173).

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In certain embodiments, the biological sample contains polypeptide molecules from the test subject. Alternatively, the biological sample can contain mRNA molecules from the test subject or genomic DNA molecules from the test subject.

In other embodiments, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

The expression of a protein can also be determined by detecting and/or measuring expression of a protein. Methods of determining protein expression generally involve the use of antibodies specific for the target protein of interest. For example, methods of determining protein expression include, but are not limited to, western blot or dot blot analysis, immunohistochemistry (e.g., quantitative immunohistochemistry), immunocytochemistry, enzyme-linked immunosorbent assay (ELISA), enzyme-linked immunosorbent spot (ELISPOT; Coligan et al., eds. (1995) Current Protocols in Immunology. Wiley, New York), or antibody array analysis (see, e.g., U.S. Patent Application Publication Nos. 20030013208 and 2004171068, the disclosures of each of which are incorporated herein by reference in their entirety). Further description of many of the methods above and additional methods for detecting protein expression can be found in, e.g., Sambrook et al. (supra).

In one example, the presence or amount of protein expression of a fusion can be determined using a western blotting technique. For example, a lysate can be prepared from a biological sample, or the biological sample itself, can be contacted with Laemmli buffer and subjected to sodium-dodecyl sulfate polyacrylamide gel electrophoresis (SDS-PAGE). SDS-PAGE-resolved proteins, separated by size, can then be transferred to a filter membrane (e.g., nitrocellulose) and subjected to immunoblotting techniques using a detectably-labeled antibody specific to the protein of interest. The presence or amount of bound detectably-labeled antibody indicates the presence or amount of protein in the biological sample.

In another example, an immunoassay can be used for detecting and/or measuring the protein expression of a protein. As above, for the purposes of detection, an immunoassay can be performed with an antibody that bears a detection moiety (e.g., a fluorescent agent or enzyme). Proteins from a biological sample can be conjugated directly to a solid-phase matrix (e.g., a multi-well assay plate, nitrocellulose, agarose, sepharose, encoded particles, or magnetic beads) or it can be conjugated to a first member of a specific binding pair (e.g., biotin or streptavidin) that attaches to a solid-phase matrix upon binding to a second member of the specific binding pair (e.g., streptavidin or biotin). Such attachment to a solid-phase matrix allows the proteins to be purified away from other interfering or irrelevant components of the biological sample prior to contact with the detection antibody and also allows for subsequent washing of unbound antibody. Here as above, the presence or amount of bound detectably-labeled antibody indicates the presence or amount of protein in the biological sample.

Methods for generating antibodies or antibody fragments specific for a protein can be generated by immunization, e.g., using an animal, or by in vitro methods such as phage display. A polypeptide that includes all or part of a target protein can be used to generate an antibody or antibody fragment. The antibody can be a monoclonal antibody or a preparation of polyclonal antibodies.

Methods for detecting or measuring gene expression can optionally be performed in formats that allow for rapid preparation, processing, and analysis of multiple samples. This can be, for example, in multi-welled assay plates (e.g., 96 wells or 386 wells) or arrays (e.g., nucleic acid chips or protein chips). Stock solutions for various reagents can be provided manually or robotically, and subsequent sample preparation (e.g., RT-PCR, labeling, or cell fixation), pipetting, diluting, mixing, distribution, washing, incubating (e.g., hybridization), sample readout, data collection (optical data) and/or analysis (computer aided image analysis) can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay. Exemplary high-throughput cell-based assays (e.g., detecting the presence or level of a target protein in a cell) can utilize ArrayScan® VTI HCS Reader or KineticScan® HCS Reader technology (Cellomics Inc., Pittsburgh, Pa.).

The term “overexpression” as used herein means an increase in the expression level of protein or nucleic acid molecule, relative to a control level. For example, a putative cancer cell may overexpress a protein (e.g., mTOR) relative to a normal cell of the same histological type from which the cancer cell evolved. Overexpression includes an increased expression of a given gene, relative to a control level, of at least 5 (e.g., at least 10, 15, 20, 25, 30, 35, 40, 45, 50, 55, 60, 65, 70, 75, 80, 85, 90, 95, 100, 110, 120, 130 140 150, 160 170, 180, 190, 200, or more) %. Overexpression includes an increased expression, relative to a control level, of at least 1.5 (e.g., at least 2, 2.5, 3, 4, 5, 6, 7, 8, 9, 10, 20, 30, 40, 50, 100, 1000 or more) fold.

Methods for detecting mTOR complex 1 (mTORC1) activity are also known in the art. For example, Ventkatesha et al. describes an assay for detecting the activity of mTOR in a cell sample, as well as assessing mTOR1 kinase activity in cells in the presence or absence of a test compound, using Western blotting techniques. (2014) Mol Cancer 13(1):259. See also, e.g., Bajer et al. (2014) Biochem Pharmacol 88(3):313-321; U.S. Pat. No. 8,658,668; and Ikenoue et al. (2009) Methods Enzymol 452:165-180, which sets forth detailed protocols for monitoring mTOR activity in tissues. Increased activity, relative to a control level, includes at least 5 (e.g., at least 10, 15, 20, 25, 30, 35, 40, 45, 50, 55, 60, 65, 70, 75, 80, 85, 90, 95, 100, 110, 120, 130 140 150, 160 170, 180, 190, 200, or more) %. Increased activity, relative to a control level, can be at least 1.5 (e.g., at least 2, 2.5, 3, 4, 5, 6, 7, 8, 9, 10, 20, 30, 40, 50, 100, 1000 or more) fold. For example, a putative cancer cell may exhibit an increased mTORC1 kinase activity relative to a normal cell of the same histological type from which the cancer cell evolved.

In some embodiments, the methods can include identifying cells (e.g., cells from a subject suspected of being, or at risk od becoming, malignant or transformed) that bear mutations in one or both of TSC1 and TSC2. Non-limiting examples of mutations (e.g., deletions, substitutions, or addition mutations) in TSC1 associated with proliferative disorders, such as tuberous sclerosis, are known in the art and described in, e.g., van Slegtenhorst et al. (1997) Science 277:805-808; Kwiatkowska et al. (1998) Ann Hum Genet 62:277-285; and Ali et al. (1998) J Med Genet 35:969-972. Non-limiting examples of mutations in TSC2 associated with proliferative disorders are described in, e.g., Green et al. (1996) Hum Genet 97:240-243; Verhoef et al. (1999) Europ J Pediat 158:284-287; Cheadle et al. (2000) Hum Genet 107:97-114; Au et al. (1999) Am J Hum Genet 65:1790-1795; van Bakel et al. (1997) Hum Molec Genet 6:1409-1414; Maheshwar et al. (1997) Hum Molec Genet 6:1991-1996; Au et al. (1998) Am J Hum Genet 62:286-294; and Green et al. (1994) Nature Genet 6:192-196.

Methods for detecting the presence of a mutation in a gene of interest are known in the art. Suitable methods for determining whether or not a particular mutation in a gene exists include, e.g., Southern blot (see, e.g., Sambrook et al. (supra)), real-time PCR analysis (see, e.g., Oliver et al. (2000) J Mol Diagnostics 2(4):202-208), nucleic acid array analysis, allele-specific PCR (e.g., quantitative allele-specific PCR), pyrosequencing, DNA sequencing (e.g., Sanger chemistry sequencing), or through the use of molecular beacons (e.g., Tyagi et al. (1998) Nat Biotechnol 16:49-53; Abravaya et al. (2003) Clin Chem Lab Med 41:468-474; and Mullah et al. (1999) Nucleos Nucleot 18:1311-1312, the disclosures of each of which are incorporated herein by reference in their entirety).

To determine a genotype using Southern blot analysis, first, genomic DNA is isolated from a biological sample from a subject (e.g., a human patient), e.g., using a detergent (e.g., NP40 and/or sodium dodecyl sulfate), and proteinase K digestion, followed by sodium chloride extraction, and ethanol wash of the extracted DNA. Regions of DNA containing the mutation of interest can be amplified using PCR. The amplicons can be subjected to gel-electrophoresis to separate the nucleic acids by size, and then transferred to a solid support such as a nitrocellulose membrane. To detect the presence of a gene mutation in the biological sample, the solid support containing the amplicons can be contacted with a detectably-labeled, complementary oligonucleotide probe that specifically hybridizes to a nucleic acid containing a mutation under appropriate stringency conditions. The binding of the probe to an amplicon indicates the presence of the corresponding nucleic acid containing the mutation in the biological sample.

In another example, a particular genotype can also be detected using nucleic acid arrays. For example, genomic DNA isolated from a biological sample can be amplified using PCR as described above. The amplicons can be detectably-labeled during the PCR amplification process (e.g., using one or more detectably labeled deoxynucleotides (dNTPs)) or subsequent to the amplification process using a variety of chemical or enzymatic techniques such as nick-translation. Following amplification and labeling, the detectably-labeled-amplicons are then contacted to a plurality of polynucleotide probe sets, each set containing one or more of a polynucleotide (e.g., an oligonucleotide) probe specific for (and capable of binding to) a corresponding amplicon, and where the plurality contains many probe sets each corresponding to a different amplicon. Generally, the probe sets are bound to a solid support and the position of each probe set is predetermined on the solid support. The binding of a detectably-labeled amplicon to a corresponding probe of a probe set indicates the presence of the gene mutation so amplified in the biological sample. Suitable conditions and methods for detecting gene mutations using nucleic acid arrays are further described in, e.g., Lamy et al. (2006) Nucleic Acids Research 34(14): e100; European Patent Application Publication No. 1234058; U.S. Patent Application Publication Nos. 20060008823 and 20030059813; and U.S. Pat. No. 6,410,231; the disclosures of each of which are incorporated herein by reference in their entirety.

Any of the methods of detecting a gene mutation can, optionally, be performed in formats that allow for rapid preparation, processing, and analysis of multiple samples (see above).

The detection of one or more of the gene mutations can use the nucleic acid sequences of the mutations themselves, and surrounding sequence, e.g., as hybridization polynucleotide probes or primers (e.g., for amplification or reverse transcription). Nucleic acid probes should contain a sequence of sufficient length and complementarity to a corresponding mutated region to specifically hybridize with that region under suitable hybridization conditions. For example, the probe can include at least one (e.g., at least two, at least three, at least four, at least five, at least six, at least seven, at least eight, at least nine, at least 10, at least 11, at least 12, at least 15, at least 20, at least 25, at least 30, at least 35, at least 40, at least 45, at least 50, or 55 or more) nucleotides 5′ or 3′ to the mutation of interest. The polymorphic site of each probe (i.e., the mutated region) is generally flanked on one or both sides by sequence that is common between the mutant and wild-type form of a gene.

Therapeutic Methods

The disclosure also features in vitro and in vivo methods for inhibiting proliferating cells, e.g., inhibiting the growth, activity, proliferation, metabolism, or viability of a proliferating cell. Methods for assessing inhibition of proliferating cells are known in the art. Yet the disclosure also features methods for treating a subject with a cell proliferative disorder, such as cancer. For example, methods disclosed herein include the use of purine synthesis inhibitors (such as those targeting IMPDH) to treat tumors with high mTOR signaling such as those in TSC and certain cancers. See e.g., Issam Ben-Sahara et al., Science 351, 728-733 (2016).

As used herein, the phrase “cell proliferative disorder” refers to a neoplasm. That is, a new, abnormal growth of cells or a growth of abnormal cells which reproduce faster than normal. A neoplasm creates an unstructured mass (a tumor) which can be either benign or malignant. The term “benign” refers to a tumor that is noncancerous, e.g., its cells do not invade surrounding tissues or metastasize to distant sites. The term “malignant” refers to a tumor that is cancerous, and/or metastastic, i.e., invades contiguous tissue or is no longer under normal cellular growth control.

In some embodiments, the cell proliferative disorder is tuberous sclerosis complex, lymphangioleiomyomatosis, a PTEN mutant hamartoma syndrome, Peutz Jeghers syndrome, Familial Adenomatous Polyposis, or neurofibromatosis type 1. The PTEN mutant hamartoma syndrome can be, e.g., Cowden disease, Proteus disease, Lhermitte-Duclos disease, or Bannayan-Riley-Ruvalcaba syndrome.

In some embodiments, the cell proliferative disorder is a cancer. After it is determined that a subject (e.g., a human) has a cancer (e.g., bearing a mutation in one or both of TSC1 and TSC2 or one having an increased level of mTORC1 activity), a medical practitioner may elect to administer to the human an anti-cancer therapy (e.g., one or more chemotherapies and/or immunotherapies). In some embodiments, the methods include diagnosing the subject as having a cancer (e.g., using any of the methods described herein) and selecting an anti-cancer therapy for the subject, e.g., based at least in part on the information provided by the diagnostic method. In some embodiments, the methods include diagnosing the subject as having a cancer (e.g., using any of the methods described herein) and administering the anti-cancer therapy to the subject, e.g., based at least in part on the information provided by the diagnostic method.

Cancer is a class of diseases or disorders characterized by uncontrolled division of cells and the ability of these to spread, either by direct growth into adjacent tissue through invasion, or by implantation into distant sites by metastasis (where cancer cells are transported through the bloodstream or lymphatic system). Cancer can affect people at all ages, but risk tends to increase with age. Types of cancers can include, e.g., lung cancer, breast cancer, colon cancer, pancreatic cancer, renal cancer, stomach cancer, liver cancer, bone cancer, hematological cancer, neural tissue cancer (e.g., neuroblastoma), melanoma, thyroid cancer, ovarian cancer, testicular cancer, prostate cancer, cervical cancer, vaginal cancer, or bladder cancer. Hematological cancers (liquid tumors) include, e.g., leukemias (e.g., chronic lymphocytic leukemia such as B cell or T cell type chronic lymphocytic leukemia) and multiple myeloma. Bone cancers include, without limitation, osteosarcoma and osteocarcinomas. Exemplary types of cancer are also set forth in Table 1.

As used herein, a “subject” is a mammal (e.g., mouse, rat, primate, non-human mammal, domestic animal such as dog, cat, cow, horse), preferably a human. As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment.

The term “preventing” is art-recognized, and when used in relation to a condition, is well understood in the art, and includes administration of a composition which reduces the frequency of, or delays the onset of, symptoms of a medical condition in a subject mammal relative to a subject which does not receive the composition.

As used herein, a subject “at risk of developing a cancer” is a subject that has a predisposition to develop a cancer, i.e., a genetic predisposition to develop cancer such as a mutation in a tumor suppressor gene (e.g., mutation in BRCA1, p53, RB, or APC) or has been exposed to conditions that can result in cancer. Thus, a human can also be one “at risk of developing a cancer” when the human has been exposed to mutagenic or carcinogenic levels of certain compounds (e.g., carcinogenic compounds in cigarette smoke such as acrolein, arsenic, benzene, benz{a}anthracene, benzo{a}pyrene, polonium-210 (radon), urethane, or vinyl chloride). Moreover, the human can be “at risk of developing a cancer” when the human has been exposed to, e.g., large doses of ultraviolet light or X-irradiation, or infected by a tumor-causing/associated virus such as a papillomavirus, Epstein-Barr virus, hepatitis B virus, or human T-cell leukemia-lymphoma virus. From the above it will be clear that humans “at risk of developing a cancer” are not all the humans within a species of interest.

A human “suspected of having a cancer” is one having one or more symptoms of a cancer. Symptoms of cancer are well-known to those of skill in the art and include, without limitation, breast lumps, pain, weight loss, weakness, excessive fatigue, difficulty eating, loss of appetite, chronic cough, worsening breathlessness, coughing up blood, blood in the urine, blood in stool, nausea, vomiting, liver metastases, lung metastases, bone metastases, abdominal fullness, bloating, fluid in peritoneal cavity, vaginal bleeding, constipation, abdominal distension, perforation of colon, acute peritonitis (infection, fever, pain), pain, vomiting blood, heavy sweating, fever, high blood pressure, anemia, diarrhea, jaundice, dizziness, chills, muscle spasms, and difficulty swallowing. Symptoms of a primary cancer (e.g., a large primary cancer) can include, e.g., any one of colon metastases, lung metastases, bladder metastases, liver metastases, bone metastases, kidney metastases, and pancreas metastases. Similarly, a skilled artisan would appreciate that a sample (e.g., a biopsy) obtained from such a subject could contain cells suspected of being cancer cells.

The compositions described herein can be administered to a subject, e.g., a human subject, using a variety of methods that depend, in part, on the route of administration. The route can be, e.g., intravenous injection or infusion (IV), subcutaneous injection (SC), intraperitoneal (IP) injection, or intramuscular injection (IM).

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; EP488401; and EP 430539, the disclosures of each of which are incorporated herein by reference in their entirety. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

As used herein the term “effective amount” or “therapeutically effective amount”′ in an in vivo setting, means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

Suitable human doses of any of the compositions described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

Toxicity and therapeutic efficacy of such compositions can be determined by known pharmaceutical procedures in cell cultures or experimental animals (e.g., animal models of cancer, vaccination, or infection). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. The dosage of such antibodies or antigen-binding fragments thereof lies generally within a range of circulating concentrations of the antibodies or fragments that include the ED₅₀ with little or no toxicity. The dosage may vary within this range depending upon the dosage form employed and the route of administration utilized. A therapeutically effective dose can be estimated initially from cell culture assays. A dose can be formulated in animal models to achieve a circulating plasma concentration range that includes the IC₅₀ (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal modeling can be used to determine a dose required to achieve a therapeutically effective concentration within the local site.

In some embodiments of any of the methods described herein, an agent can be administered to a mammal in conjunction with one or more additional therapeutic agents.

Suitable additional anti-cancer therapies include, e.g., chemotherapeutic agents, ionizing radiation, immunotherapy agents, or hyperthermotherapy. Chemotherapeutic agents include, but are not limited to, aminoglutethimide, amsacrine, anastrozole, asparaginase, bcg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, taxol, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic anti-tumor compounds may be categorized by their mechanism of action into groups, including, for example, the following: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, mechlorethamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); immunomodulatory agents (thalidomide and analogs thereof such as lenalidomide (Revlimid, CC-5013) and CC-4047 (Actimid)), cyclophosphamide; anti-angiogenic compounds (TNP-470, genistein) and growth factor inhibitors (vascular endothelial growth factor (VEGF)-inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

The term “immunotherapeutic agent” can include any molecule, peptide, antibody or other agent which can stimulate a host immune system to generate an immune response to a tumor or cancer in the subject. Various immunotherapeutic agents are useful in the compositions are known in the art and include, e.g., PD-1 and/or PD-1L inhibitors, CD200 inhibitors, CTLA4 inhibitors, and the like. Exemplary PD-1/PD-L1 inhibitors (e.g., anti-PD-1 and/or anti-PD-L1 antibodies) are known in the art and described in, e.g., International Patent Application Publication Nos. WO 2010036959 and WO 2013/079174, as well as U.S. Pat. Nos. 8,552,154 and 7,521,051, the disclosures of each of which as they relate to the antibody descriptions are incorporated herein by reference in their entirety. Exemplary CD200 inhibitors are also known in the art and described in, e.g., International Patent Application Publication No. WO 2007084321. Suitable anti-CTLA4 antagonist agents are described in International Patent Application Publication Nos. WO 2001/014424 and WO 2004/035607; U.S. Patent Application Publication No. 2005/0201994; and European Patent No. EP 1212422. Additional CTLA-4 antibodies are described in U.S. Pat. Nos. 5,811,097, 5,855,887, 6,051,227, and 6,984,720;

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In some embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In some embodiments, hyperthermia, a procedure in which body tissue is exposed to high temperatures (up to 106° F.) is used to treat the cancer or is selected as a therapy for the subject. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In some embodiments, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about three centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early nonsmall cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In some embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser: This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser: light from this laser can penetrate deeper into tissue than light from the other types of lasers, and it can cause blood to clot quickly. It can be carried through optical fibers to less accessible parts of the body. This type of laser is sometimes used to treat throat cancers. Argon laser: this laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

In some embodiments, the compounds described herein can be used for treating a subject having an autoimmune or inflammatory disorder (e.g., an acute or chronic condition). In some embodiments, the inflammatory disorder can be, e.g., acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barre syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjogren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behcet's syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease, or combinations of any of the foregoing. In some embodiments, the autoimmune or inflammatory disorder can be, e.g., colitis, multiple sclerosis, arthritis, rheumatoid arthritis, osteoarthritis, juvenile arthritis, psoriatic arthritis, acute pancreatitis, chronic pancreatitis, diabetes, insulin-dependent diabetes mellitus (IDDM or type I diabetes), insulitis, inflammatory bowel disease, Crohn's disease, ulcerative colitis, autoimmune hemolytic syndromes, autoimmune hepatitis, autoimmune neuropathy, autoimmune ovarian failure, autoimmune orchitis, autoimmune thrombocytopenia, reactive arthritis, ankylosing spondylitis, silicone implant associated autoimmune disease, Sjogren's syndrome, systemic lupus erythematosus (SLE), vasculitis syndromes (e.g., giant cell arteritis, Behcet's disease, and Wegener's granulomatosis), vitiligo, secondary hematologic manifestation of autoimmune diseases (e.g., anemias), drug-induced autoimmunity, Hashimoto's thyroiditis, hypophysitis, idiopathic thrombocytic pupura, metal-induced autoimmunity, myasthenia gravis, pemphigus, autoimmune deafness (e.g., Meniere's disease), Goodpasture's syndrome, Graves' disease, HIV-related autoimmune syndromes and Gullain-Barre disease.

In some embodiments, autoimmune or inflammatory disorder is a hypersensitivity reaction. As used herein, “hypersensitivity” refers to an undesirable immune system response. Hypersensitivity is divided into four categories. Type I hypersensitivity includes allergies (e.g., Atopy, Anaphylaxis, or Asthma). Type II hypersensitivity is cytotoxic/antibody mediated (e.g., Autoimmune hemolytic anemia, Thrombocytopenia, Erythroblastosis fetalis, or Goodpasture's syndrome). Type III is immune complex diseases (e.g., Serum sickness, Arthus reaction, or SLE). Type IV is delayed-type hypersensitivity (DTH), Cell-mediated immune memory response, and antibody-independent (e.g., Contact dermatitis, Tuberculin skin test, or Chronic transplant rejection). As used herein, “allergy” means a disorder characterized by excessive activation of mast cells and basophils by IgE. In certain instances, the excessive activation of mast cells and basophils by IgE results (either partially or fully) in an inflammatory response. In certain instances, the inflammatory response is local. In certain instances, the inflammatory response results in the narrowing of airways (i.e., bronchoconstriction). In certain instances, the inflammatory response results in inflammation of the nose (i.e., rhinitis). In certain instances, the inflammatory response is systemic (i.e., anaphylaxis).

In some embodiments, the compounds described herein can be used to treat a neurological or neurodegenerative condition, such as an autism spectrum disorder (e.g., autism) or epilepsy.

The following examples are meant to exemplify, not to limit, the disclosure.

### EXAMPLES

### Example 1. Materials and Methods

### Generation of CRISPR Expression Vector

A Drosophila codon optimized Cas9 with 3× Flag tag and NLS elements at both 5′ and 3′ was synthesized by GenScript and the Drosophila U6 promoter and act5c promoter were PCR amplified from fly genomic DNA (Table 1). These were used to replace the human codon optimized Cas9, human U6 and CGh promoters respectively of the px330 (13) plasmid to yield the pl018 plasmid. sgRNA homology sequences were cloned into pl018 using pairs of DNA oligonucleotides, which were annealed and ligated into BbsI sites according to a previously described protocol (13) (Table 1).

TABLE 1
Primers
Primers for HRM assays

| | |
|---|---|
| y_invivo_L | CACGGTCCACAGAAGAGGAT (SEQ ID NO: 23) |
| y_invivo_R | TTAGTCGGGTATTCGGGAAA (SEQ ID NO: 24) |
| y_invivo_HRM_L | TGCCAACACACTTTGGCTTA (SEQ ID NO: 25) |
| y_invivo_HRM_R | AGGTGATCAGGGTCACAAGG (SEQ ID NO: 26) |
| y_cells_L | CACGGTCCACAGAAGAGGAT (SEQ ID NO: 27) |
| y_cells_R | TTAGTCGGGTATTCGGGAAA (SEQ ID NO: 28) |
| y_cells_HRM_L | CCTTGTGACCCTGATCACCT (SEQ ID NO: 29) |
| y_cells_HRM_R | TTAGTCGGGTATTCGGGAAA (SEQ ID NO: 30) |

Primers for vector construction

| | |
|---|---|
| actin5c_L | ctagtctagaggtaccCAGCATGCAATTCTATATTC (SEQ ID NO: 31) |
| actin5c_R | ctagaccggtATCTGGATCCGGGGTCTCTG (SEQ ID NO: 32) |
| U6b_L | acgtGGTCTCacatgtGTTCGACTTGCAGCCTGAAATA (SEQ ID NO: 33) |
| U6b_R | acgtGGTCTCgaaacagGTCTTCtcGAAGACccGAACgaagtattgaggaaaacatac (SEQ ID NO: 34) |
| MT_L | tagtGGTCTCaGACCgttgcaggacaggatgtg (SEQ ID NO: 35) |
| MT_R | tagtGGTCTCaCTGACATtttagttgcactgagatg (SEQ ID NO: 36) |
| luc_L | tagtGGTCTCaTTGGaagacgccaaaaacat (SEQ ID NO: 37) |
| luc_R | tagtGGTCTCaTATAttttacggttcctggccttt (SEQ ID NO: 38) |

Primers for long homology vector cloning

| | |
|---|---|
| ex_L_5 | gcatGGTCTCtGACCagcatccgtctctcggtcta (SEQ ID NO: 39) |
| ex_R_5 | gcatGGTCTCtATCCgctgcatcattttccgtataca (SEQ ID NO: 40) |
| ex_L_3 | gcatGGTCTCtCGAAgagcattttgcaccgtcag (SEQ ID NO: 41) |
| ex_R_3 | gcatGGTCTCtTATAtcctctctcctttcttgtttgc (SEQ ID NO: 42) |
| Myo_L_5 | gcatGGTCTCtGACCtcccaaatcgaaagcttgtt (SEQ ID NO: 43) |
| Myo_R_5 | gcatGGTCTCtATCCggtgtccttttgatcctgc (SEQ ID NO: 44) |
| Myo_L_3 | gcatGGTCTCtCGAActggagtgcaggacttcgtc (SEQ ID NO: 45) |
| Myo_R_3 | gcatGGTCTCtTATAttgtggctgcgtggttgt (SEQ ID NO: 46) |

### Luciferase-Based Mutation Reporter Assays

The luciferase reporter vector was constructed by PCR amplifying the metallothionein promoter from pMK33 and luciferase gene from pGL3 (Table 1) and combining these with annealed oligos containing a gRNA target site (Table 1 and Table 2) and a custom made cloning vector using Golden Gate assembly.

TABLE 2
gRNA Efficiency Analysis Data

| | GC % | GC % | GC % | Mean | |
|---|---|---|---|---|---|
| gRNA sequence | total | 6PAMP | 4PAMP | efficiency | SEM |
| CTCACAGACGGCGCCCGGCG | 80 | 100 | 100 | 0.474112001 | 0.02116738 |
| (SEQ ID NO: 47) | | | | | |
| GCCCAGGGGGAATCGGCCGC | 80 | 100 | 100 | 0.407225405 | 0.01158044 |
| (SEQ ID NO: 48) | | | | | |
| CCGCGCCGAGGACTACCGCC | 80 | 83.3333333 | 100 | 0.456826656 | 0.01400356 |
| (SEQ ID NO: 49) | | | | | |
| AAAGAGCTGCGGGCCGGGCG | 75 | 100 | 100 | 0.069536738 | 0.00334651 |
| (SEQ ID NO: 50) | | | | | |
| CGTCGTTCTCGCCACCGCGC | 75 | 100 | 100 | 0.146099847 | 0.00631515 |
| (SEQ ID NO: 51) | | | | | |
| ACGCGCACGGGTCTCACGCC | 75 | 83.3333333 | 100 | 0.328751416 | 0.01433175 |
| (SEQ ID NO: 52) | | | | | |
| GGCACCGGGTAGCCTTGCGC | 75 | 66.6666667 | 100 | 0.349715624 | 0.03393357 |
| (SEQ ID NO: 53) | | | | | |
| CTGGGCCGGCGTGTCCGTTC | 75 | 66.6666667 | 50 | 0.432837943 | 0.00425029 |
| (SEQ ID NO: 54) | | | | | |
| GGGCTCCAGGCTGCGCGCAA | 75 | 66.6666667 | 50 | 0.230824794 | 0.00978178 |
| (SEQ ID NO: 55) | | | | | |
| CGCCGGCACCGCGTCCCTAT | 75 | 50 | 25 | 0.254206107 | 0.0070367 |
| (SEQ ID NO: 56) | | | | | |
| CAACCAACAGGTCCGGCCCC | 70 | 100 | 100 | 0.383313341 | 0.02921678 |
| (SEQ ID NO: 57) | | | | | |
| AGGGAGTCCGCATAGGCGGC | 70 | 100 | 100 | 0.181907213 | 0.00435846 |
| (SEQ ID NO: 58) | | | | | |
| TCGCGGGCTTGCATGACGCG | 70 | 83.3333333 | 100 | 0.329481952 | 0.00753257 |
| (SEQ ID NO: 59) | | | | | |
| TGACGCCTCAGGGTGCCGTC | 70 | 83.3333333 | 75 | 0.65942659 | 0.0077931 |
| (SEQ ID NO: 60) | | | | | |
| ACCGAAAGTCGCGCCGGACG | 70 | 83.3333333 | 75 | 0.249561279 | 0.01729587 |
| (SEQ ID NO: 61) | | | | | |
| AGTCCTCACCGTGGGGCACC | 70 | 83.3333333 | 75 | 0.090691763 | 0.00332196 |
| (SEQ ID NO: 62) | | | | | |
| CAGCCTCTCCCTGCGGATGC | 70 | 66.6666667 | 50 | 0.250913216 | 0.0261434 |
| (SEQ ID NO: 63) | | | | | |
| CCTGGATGATACGCTGGCGC | 65 | 83.3333333 | 100 | 0.180709738 | 0.00460631 |
| (SEQ ID NO: 64) | | | | | |
| GCATGCCCACCTTATCGGCG | 65 | 83.3333333 | 100 | 0.116921695 | 0.00677685 |
| (SEQ ID NO: 65) | | | | | |
| CAGGCATTGTGACGACGCGC | 65 | 83.3333333 | 100 | 0.17438184 | 0.01173932 |
| (SEQ ID NO: 66) | | | | | |
| TTGACATCAGCGCGCGAGGC | 65 | 83.3333333 | 75 | 0.389342941 | 0.02064832 |
| (SEQ ID NO: 67) | | | | | |
| ACCAGCATATGCCGGCCTGC | 65 | 83.3333333 | 75 | 0.76698655 | 0.02200554 |
| (SEQ ID NO: 68) | | | | | |
| TGTTCCCTACACGGCGCCAG | 65 | 83.3333333 | 75 | 0.253098468 | 0.01387223 |
| (SEQ ID NO: 69) | | | | | |
| GGATGTTCCGGCACAACGCC | 65 | 66.6666667 | 100 | 0.275327255 | 0.0154501 |
| (SEQ ID NO: 70) | | | | | |
| GTGTCCAGGCTCGTCATGCC | 65 | 66.6666667 | 75 | 0.860320032 | 0.05683693 |
| (SEQ ID NO: 71) | | | | | |
| CCTTCGCGAGCACCCGGAAA | 65 | 50 | 25 | 0.381172456 | 0.02322788 |
| (SEQ ID NO: 72) | | | | | |
| CCGCCGTGTGCTGCGAATAC | 65 | 33.3333333 | 25 | 0.166092642 | 0.00150101 |
| (SEQ ID NO: 73) | | | | | |
| GGTTACCAACTCAGGACCCG | 60 | 83.3333333 | 100 | 0.485759532 | 0.04422996 |
| (SEQ ID NO: 74) | | | | | |
| AGACTGGTTTCCACCCGCTC | 60 | 83.3333333 | 75 | 1.297751273 | 0.07376942 |
| (SEQ ID NO: 75) | | | | | |
| GGAATAACCACGTCCCGCGT | 60 | 83.3333333 | 75 | 0.28938268 | 0.0059461 |
| (SEQ ID NO: 76) | | | | | |
| CCACCGCGATATATGGGCGA | 60 | 83.3333333 | 75 | 0.571102349 | 0.01848316 |
| (SEQ ID NO: 77) | | | | | |
| GACCGGATTCGCTACAACCC | 60 | 66.6666667 | 75 | 0.696260208 | 0.05748289 |
| (SEQ ID NO: 78) | | | | | |
| CAAGCAAGTCCGGTCGGTTG | 60 | 66.6666667 | 50 | 0.35314738 | 0.00738644 |
| (SEQ ID NO: 79) | | | | | |
| ATATCGGAGTCGGCCCAACC | 60 | 66.6666667 | 50 | 0.411323524 | 0.00915308 |
| (SEQ ID NO: 80) | | | | | |
| GCTCTGGACCATACGGGGAT | 60 | 66.6666667 | 50 | 0.174647152 | 0.01096014 |
| (SEQ ID NO: 81) | | | | | |
| TGCGCTTACCGACGATTGGG | 60 | 50 | 75 | 0.110910151 | 0.0079577 |
| (SEQ ID NO: 82) | | | | | |
| CTTCCGACCCTTCGTTAGCC | 60 | 50 | 75 | 0.181521344 | 0.01152974 |
| (SEQ ID NO: 83) | | | | | |
| ACCGCCAAACGGACCGCTTA | 60 | 50 | 25 | 0.310523009 | 0.00891293 |
| (SEQ ID NO: 84) | | | | | |
| CCGTCGAAGAACCGCGCAAT | 60 | 50 | 25 | 1.029402815 | 0.08513755 |
| (SEQ ID NO: 85) | | | | | |
| TCGATGACGCCCGGGTTAAC | 60 | 33.3333333 | 25 | 0.260025073 | 0.00908501 |
| (SEQ ID NO: 86) | | | | | |
| TCCTATTACTGCGTGCAGCC | 55 | 83.3333333 | 75 | 1.342145722 | 0.06209031 |
| (SEQ ID NO: 87) | | | | | |
| GCGAGTTATCTATCCCCAGG | 55 | 83.3333333 | 75 | 0.474912047 | 0.02240853 |
| (SEQ ID NO: 88) | | | | | |
| GGAGCGAGTTATCTATCCCC | 55 | 66.6666667 | 100 | 0.061812861 | 0.00401223 |
| (SEQ ID NO: 89) | | | | | |
| AACGACATCACGGTCCATGC | 55 | 66.6666667 | 50 | 0.242354092 | 0.01388622 |
| (SEQ ID NO: 90) | | | | | |
| GGTTCACTATTGGGGCGATC | 55 | 66.6666667 | 50 | 0.177149437 | 0.01040299 |
| (SEQ ID NO: 91) | | | | | |
| ATCTTCCCGTCGACATAGCC | 55 | 50 | 75 | 0.16088364 | 0.00985631 |
| (SEQ ID NO: 92) | | | | | |
| ATCTGGTCAGGACGCAAAGC | 55 | 50 | 50 | 0.140599012 | 0.01076411 |
| (SEQ ID NO: 93) | | | | | |
| TCGAGGATGGGACGCTTATC | 55 | 33.3333333 | 25 | 0.505284878 | 0.02452416 |
| (SEQ ID NO: 94) | | | | | |
| CGACGTTCCTGGCTCAATTG | 55 | 33.3333333 | 25 | 0.906732313 | 0.03223828 |
| (SEQ ID NO: 95) | | | | | |
| TTGGCCGCGACCTGTTTATC | 55 | 16.6666667 | 25 | 0.928241634 | 0.01692274 |
| (SEQ ID NO: 96) | | | | | |
| GTGCTGCGCCAACCCAAATA | 55 | 16.6666667 | 0 | 0.084904618 | 0.00516422 |
| (SEQ ID NO: 97) | | | | | |
| CGAATTACTACGTAGAGCGG | 50 | 83.3333333 | 100 | 0.111688629 | 0.01110238 |
| (SEQ ID NO: 98) | | | | | |
| TAAGACATACGTACGCCCAG | 50 | 83.3333333 | 75 | 0.181350319 | 0.00564994 |
| (SEQ ID NO: 99) | | | | | |
| TTGTTCGAAGATCTGCGCGA | 50 | 83.3333333 | 75 | 0.221185865 | 0.00842408 |
| (SEQ ID NO: 100) | | | | | |
| ATAGTTCCCTCCATGCCCTT | 50 | 66.6666667 | 50 | 0.077864168 | 0.0042067 |
| (SEQ ID NO: 101) | | | | | |
| CCAGTAACGTCAAAGCTAGG | 50 | 66.6666667 | 50 | 0.106795782 | 0.00618705 |
| (SEQ ID NO: 102) | | | | | |
| AACTCAAAGACCCGCTTTCG | 50 | 50 | 50 | 0.140756985 | 0.00569506 |
| (SEQ ID NO: 103) | | | | | |
| AAGTAGGCTCTCCTGAATGG | 50 | 50 | 50 | 0.122415605 | 0.00263538 |
| (SEQ ID NO: 104) | | | | | |
| GTCGGAACCTGAGTCGTTTT | 50 | 33.3333333 | 0 | 0.589086648 | 0.00803604 |
| (SEQ ID NO: 105) | | | | | |
| ATCTGACCTCCCGGCTAATT | 50 | 16.6666667 | 0 | 0.057366313 | 0.00143433 |
| (SEQ ID NO: 106) | | | | | |
| TGCGGTCGCGTGAGAAATAT | 50 | 0 | 0 | 0.028758274 | 0.00154628 |
| (SEQ ID NO: 107) | | | | | |
| TCGTACCGCACCGGTATTTT | 50 | 0 | 0 | 0.156451933 | 0.00599576 |
| (SEQ ID NO: 108) | | | | | |
| AAACAGAAGGTCTTCCCCAA | 45 | 66.6666667 | 50 | 0.304562105 | 0.02441574 |
| (SEQ ID NO: 109) | | | | | |
| TTGTTTGCTGGTACAAACGC | 45 | 50 | 75 | 0.29932773 | 0.01617132 |
| (SEQ ID NO: 110) | | | | | |
| GTGGAATCTACTGTGGAATC | 45 | 50 | 25 | 0.296856138 | 0.02207256 |
| (SEQ ID NO: 111) | | | | | |
| ACCCTGTCCACTCATATATG | 45 | 16.6666667 | 25 | 0.417200782 | 0.01762459 |
| (SEQ ID NO: 112) | | | | | |
| GCCCGAAAGACACGTTTTTT | 45 | 0 | 0 | 0.19700112 | 0.00652626 |
| (SEQ ID NO: 113) | | | | | |
| GCACCCTGCACGAATTATTT | 45 | 0 | 0 | 0.204079133 | 0.01225648 |
| (SEQ ID NO: 114) | | | | | |
| TTCTGAGTACATGTGATACC | 40 | 50 | 50 | 0.297365049 | 0.00546395 |
| (SEQ ID NO: 115) | | | | | |
| GACAAAGGATACTCAAATCC | 40 | 33.3333333 | 50 | 0.051690659 | 0.00331017 |
| (SEQ ID NO: 116) | | | | | |
| TTCTCCGCTGTTATCCTATA | 40 | 33.3333333 | 0 | 0.08181554 | 0.00430936 |
| (SEQ ID NO: 117) | | | | | |
| CTGCCGTTCTTCAGAAATAT | 40 | 0 | 0 | 0.310109132 | 0.02449807 |
| (SEQ ID NO: 118) | | | | | |
| GAACTCCGACTAGATTATAT | 35 | 0 | 0 | 0.40121327 | 0.02224543 |
| (SEQ ID NO: 119) | | | | | |
| TTTTTTTGGGATCATATACG | 30 | 33.3333333 | 50 | 0.12781853 | 0.01274857 |
| (SEQ ID NO: 120) | | | | | |
| AAGATAAGTTCTCTCCTATT | 30 | 33.3333333 | 0 | 0.045944478 | 0.00044458 |
| (SEQ ID NO: 121) | | | | | |

Luciferase assays were performed by transfecting S2R+ cells with the relevant pl018 plasmid, luciferase reporter and pRL-TK (Promega) (to allow normalization of transfection efficiencies between samples) in 96 well plates using Effectene Transfection Reagent (Qiagen) according to the manufacturer's recommendations. Twenty-four hours after transfection, CuSO₄ was added to the cell media at a final concentration of 140 μM and cells were incubated for a further 16 hours.

Firefly and Renilla luciferase readings were taken using the Dual-Glo Luciferase Assay System (Promega) according to manufacturer's instructions and a SpectraMax Paradigm Multi-Mode Microplate Detection Platform (Molecular Devices).

### Generation of Mutant Cell Lines

Transfections

Cells were transfected using Effectene Transfection Reagent (Qiagen) according to manufacturers instructions. For generation of mutant cell lines, 360 ng of pl018 plasmid was used in conjunction with 40 ng actin-GFP plasmid as a marker of transfected cells. Transfections were performed in 6 well plates and unless stated otherwise, were incubated for four days at 25° C. before further processing.

Conditioned Media

S2R+ cells were incubated with fresh Schneider's media supplemented with 10% FBS for 16 hours while in log phase growth. Media was then filtered to remove cells and diluted 50% using fresh media supplemented with FBS to obtain the required final FBS concentration.

Single-Cell Cloning

Cloning of single cells was performed using fluorescence activated cell sorting (FACS) of GFP marked cells. Untransfected cells were used to determine background fluorescence levels before selecting the top 10% of GFP-expressing cells for isolation. Individual cells were sorted into 96 well plates containing culture media. Following two or three weeks of culture, single cells clones were identified visually and isolated into larger cultures.

HRM Assays

PCR fragments were prepared from genomic DNA as described for sequencing analysis. Reaction products were then diluted 1:10,000 before an additional round of PCR amplification using Precision Melt Supermix (Bio-Rad) and nested primers to generate a product <120 bp in length (95° C. 3 min, 50 rounds of [95° C. 30 sec, 60° C. 18 s, plate read], 95° C. 30 sec, 25° C. 30 sec, 10° C. 30 sec, 55° C. 31 sec, ramp from 55° C. to 95° C. and plate read every 0.1° C.). Data was analyzed using HRMAnalyzer Housden et al. (2014) Methods Enzymol 546:415-439. See Table 1 for primer sequences.

Sequence Verification of Clones

Genomic DNA was prepared from cultured cells by resuspension in 100 μL of lysis buffer (10 mM Tris-HCL pH8.2, 1 mM EDTA, 25 mM NaCl and 200 μg/ml proteinase K) and incubation in a thermo cycler for 1 hour at 50° C. followed by denaturation at 98° C. for 30 minutes.

Target sequences were cloned by PCR using Phusion high-fidelity DNA polymerase (NEB) according to manufacturer's recommendations and supplemented with an additional 2.5 mM MgCl₂ (35 cycles: 96° C., 30 seconds (s); 50° C., 30 s; 72° C., 30 s). PCR products were gel purified, cloned into the pCR-Blunt II-TOPO vector (Invitrogen) and transformed into Top10 chemically competent cells (Invitrogen). Following transformation, single colonies were isolated for sequencing. To assess homozygosity of single-cell samples, a minimum of 5 colonies were sequenced per sample. For identification of mutant cell lines a minimum of 20 colonies were analyzed.

### Cell Characterization Assays

Analysis of STAT92E Activity

S2R+ and STAT92E cell lines were transfected using Effectene Transfection Reagent (Qiagen) according to the manufacturer's instructions to introduce os cDNA cloned into pMK33 expression vector, Renilla expression vector (pRL-TK, promega) and 10×-STAT-luc (49) into experimental samples or pMK33, pRL-TK and 10×-STAT-luc into control samples. RNAi samples included an additional 50 ng of dsRNA (DRSC ID: DRSC16870 or DRSC37655) from the dsRNA template collection at the Drosophila RNAi Screening Center (DRSC). Cells were transfected for 24 hours before addition of CuSO₄ at a final concentration of 140 μM and incubation for a further 16 hours. Firefly and Renilla luciferase measurements were performed using a SpectraMax Paradigm Multi-Mode Microplate Detection Platform (Molecular Devices).

Homologous Recombination Experiments

Wild type S2R+ or Lig4 mutant cells were transfected with sgRNAs cloned into pl018 targeting the ex or Myo31DF genes and donor constructs containing GFP coding sequence flanked by 1 kb homology arms (Table 1). Cells were transfected for 4 days before analysis of GFP expression using a BD Biosciences LSR Fortessa X-20 cell analyzer.

Cell Size Assays

S2R+, TSC1 and TSC2 mutant cell lines were analyzed using a BD Biosciences LSR Fortessa X-20 cell analyzer to measure forward scatter for each cell as a proxy for cell diameter.

Cell Line Growth Assays

5000 cells for each line were seeded into 384 well plates containing 50 μl culture media and incubated at 25° C. for 5 days. 27 μl of CellTiter-Glo reagent (Promega) was added to each well before reading luminescence using a SpectraMax Paradigm Multi-Mode Microplate Detection Platform (Molecular Devices).

In-Cell Western Blotting

Cells were starved in FBS free Schneider's media for 24 hours in 384-well plates before fixing in 4% formaldehyde in PBS for 20 minutes at room temperature. To permeabilize, fixing solution was removed and cells were washed three times with 1×PBS containing 0.1% triton (PBX) for 10 minutes per wash. Triton washing buffer was removed and cells were blocked with PBX+5% BSA solution (PBT) for one hour. Cells were incubated with primary antibodies (1:200) in PBT with gentle agitation at 4° C. overnight. Next, cells were washed with PBT three times for 10 minutes per wash at room temperature before incubation with secondary antibody solutions (1:200) in PBT for two hours. Cells were finally washed three times with PBT for 20 minutes per wash and placed in PBS for imaging and quantification on a Li-cor Aerius system. Primary antibody was p-S6k (T398) (Cell Signaling Technology) and secondary antibody was Alexa Fluor 680 goat anti-rabbit (Invitrogen). p-S6k levels were normalized to tubulin to control for cell number.

Quantitative Phosphoproteomics

Phosphoproteomic analysis was performed as described previously (50). Briefly, S2R+, TSC1 or TSC2 mutant cells were serum starved for 16 hours before lysis in 8M urea. Samples were then digested with trypsin, peptides chemically labeled with TMT Isobaric Mass Tags (Thermo Scientific), separated into 12 fractions by strong cation exchange (SCX) chromatography, purified with TiO₂ microspheres and analyzed via LC-MS/MS on an Orbitrap Velos Pro mass spectrometer (Thermo Scientific). Peptides were identified by Sequest and filtered to a 1% peptide FDR. Proteins were filtered to achieve a 2% final protein FDR (final peptide FDR near 0.15%). TMT reporter ion intensities for individual phosphopeptides were normalized to the summed reporter ion intensity for each TMT label. The localizations of phosphosites were assigned using the AScore algorithm.

### Synthetic Screening

S2R+, TSC1 and TSC2 mutant cell lines were each screened in triplicate using the ‘kinases and phosphatases’ sub-library provided by the Drosophila RNAi Screening Center (DRSC). Screening was performed following standard procedures as described by the DRSC. Briefly, for each 384 well plate, 5000 cells in 10 ul FBS free media were seeded into each well, already containing 5 ul of dsRNA at a concentration of 50 ng/ul. Samples were incubated at room temperature for 45 minutes before adding 35 ul of 14% FBS media (bringing final FBS concentration to 10%). Plates were incubated at 25 C for five days before assaying ATP levels using CellTiter glo assays (Promega) and a SpectraMax Paradigm Multi-Mode Microplate Detection Platform (Molecular Devices).

CellTiter glo data was analyzed by normalizing data to the median value of each column (to correct for pipetting errors) and calculating z-scores for each trial individually. Z-scores greater than 1.5 or less than −1.5 in at least two out of three trials were considered to affect cell viability significantly. Synthetic lethal hits were identified as dsRNAs that significantly affect viability of TSC1 or TSC2 mutant cell lines but not S2R+.

### Validation of Synthetic Interactions in Mammalian Cells

tsc2+/+;tp53−/− and tsc2−/−;tp53−/− MEFs (51) and TSC2 deficient angiomyolipoma cells with empty vector or TSC2 addback (52) were transfected with siGENOME SMARTpool siRNAs (Dharmacon) targeting CCNT1, RNGTT or CDK11 using Lipofectamine RNAiMAX Transfection Reagent (Invitrogen) according to manufacturers reverse transfection protocol. ATP levels were quantified using the CellTiter-Glo Luminescent Cell Viability Assay (Promega) according to manufacturers instructions. The following antibodies were purchased from Cell Signaling Technology and used for western blot analysis: TSC2 #3612, phospho-T389 S6 Kinase #9234, S6 Kinase #2708, GAPDH #5174, CCNT1 #8744, CDK11 #5524. RNGTT antibody was purchased from Novus Biologicals #NBP1-49972.

### Example 2. Optimization of the CRISPR System for Drosophila Cell Culture

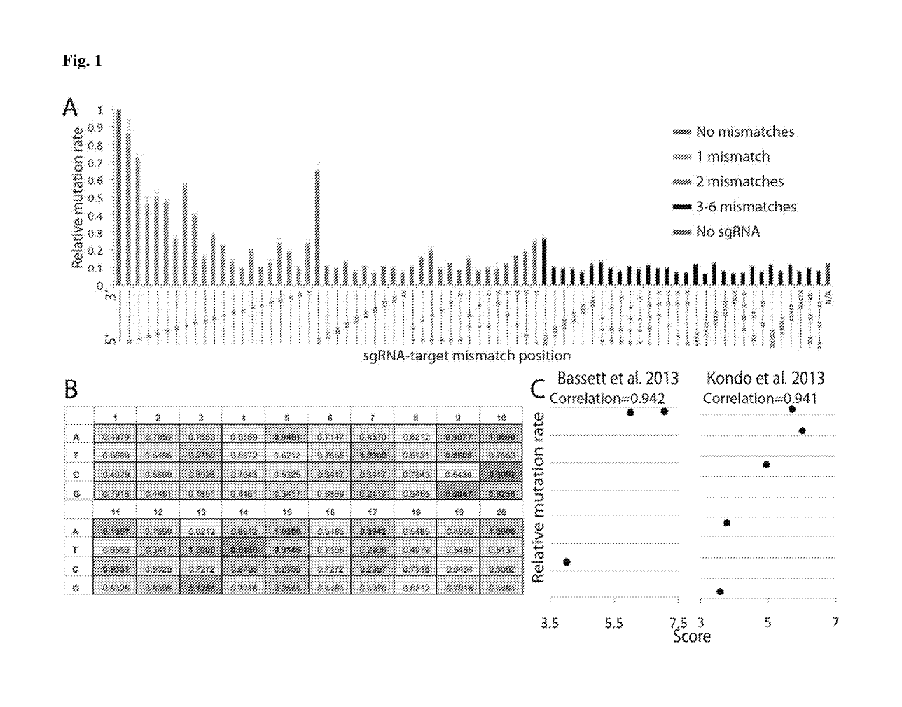

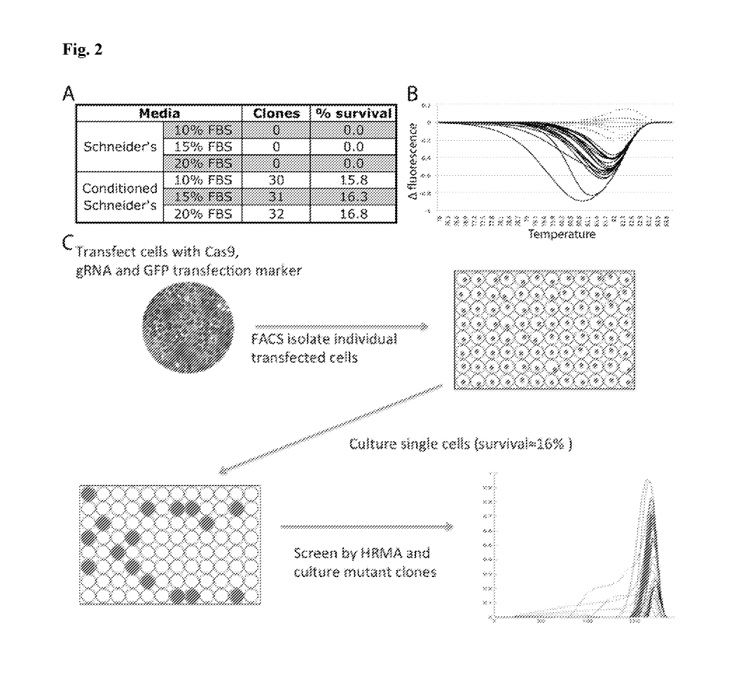

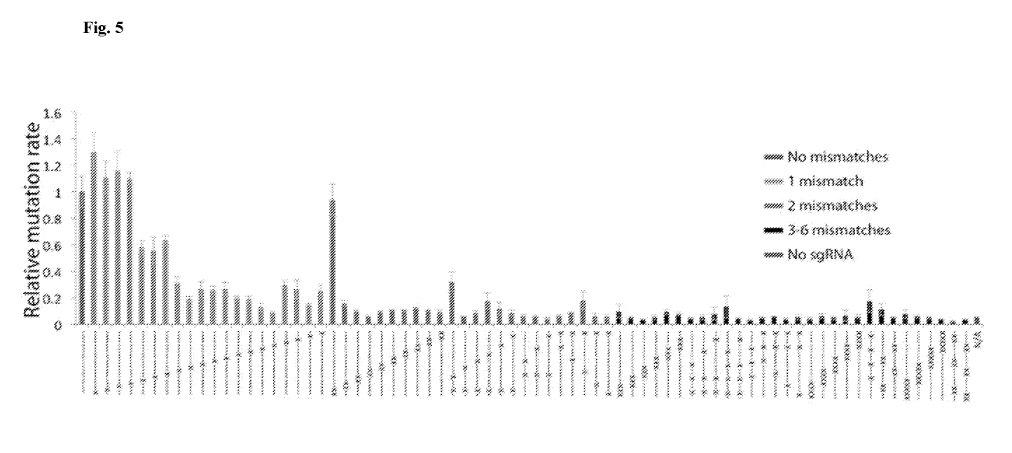

To assess the specificity of CRISPR in Drosophila cell culture, a vector encoding both Cas9 and sgRNA was generated, and then used this to express 75 variants of an sgRNA in S2R+ cells with different mismatches to a single target sequence present in a luciferase based reporter or in the genome. Two independent quantitative readouts of mutation rate were used to determine the extent and position of mismatch required to prevent mutation either in the reporter or endogenous sequence (FIGS. 1A and 5). Mismatches at the 5′ end of the sgRNA sequences were better tolerated than at the 3′ end. However, in some cases, a single mismatch was sufficient to prevent detectable mutation. In addition, it was observed that three mismatches were sufficient to prevent detectable mutations except when all mismatches were at the 5′ end of the sgRNA. Therefore 3 bp of mismatch was used as a cutoff to annotate predicted off-targets for all possible sgRNAs in the Drosophila genome. Using these updated off-target predictions, it was estimated that 97% of genes in the Drosophila genome can be targeted with specific sgRNAs, making this an ideal system for the generation of knockout cell lines.

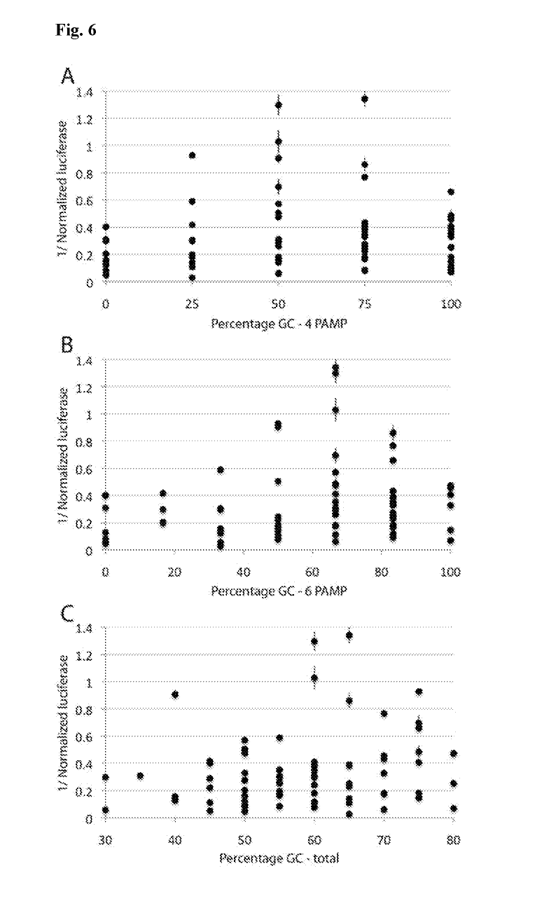

Because the rate of mutations varies widely between different sgRNAs (25-27), whether efficiency could be predicted based on the sgRNA sequence was tested. 75 additional sgRNAs were generated, each targeting luciferase-based reporter constructs with no mismatches and tested mutation efficiency for each. Using this panel of sgRNAs and associated efficiencies, the presence or absence of a correlation between GC content and mutation rate was examined. Such correlation was evident for any part of the sgRNA sequence (FIG. 6). Next, whether a more general sequence-based approach was tested for its ability to improve efficiency prediction. The nucleotide content of all 75 sgRNAs was examined, considering each position separately and generated a probability matrix linking nucleotide content with mutation rate (FIG. 1, panel B). This was then used to generate predicted efficiency scores based on sgRNA sequence. To test the performance of this approach, scores were generated for sgRNAs used in two previous Drosophila publications and found a strong correlation with reported efficiencies (FIG. 1, panel C). Note that sgRNAs targeting close to the 3′ end of genes or with apparent viability effects were not included in this analysis. Finally, predicted scores were generated for all sgRNA target sites in the Drosophila genome and annotated.

### Example 3. Generation of Stable Mutant Cell Lines

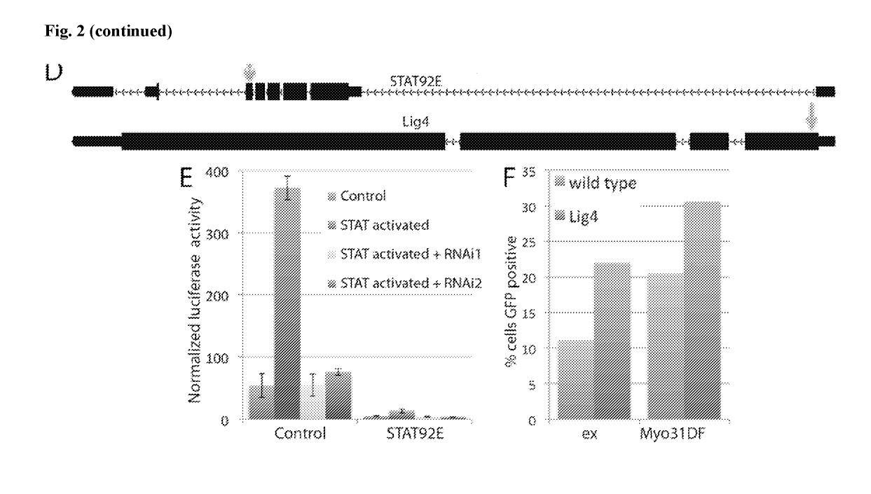

To generate cell lines in which all cells are null mutants for the target gene, optimized sgRNAs were generated using the criteria described above to maximize efficiency and minimize off-targets. Second, to ensure mutant cell lines do not revert to wild-type, a method was developed to grow cultures from individual cells, a historically difficult problem with Drosophila cells. Various methods for this have been proposed (30-32), but none have been widely used due to either difficulty in identifying single cell derived cultures or very low efficiencies. To substitute for paracrine factors that promote the survival of individual Drosophila cells cultured in populations, whether the use of culture media preconditioned using wild-type S2R+ cells would allow the efficient growth of individual S2R+ cells isolated by flow cytometry was tested. When seeded into regular media, 0/190 individual cells formed colonies but when seeded into conditioned media, 30/190 (16%) formed colonies that could be expanded into clonal cultures (FIG. 2, panel A). Variation of FBS concentration had no additional effect.

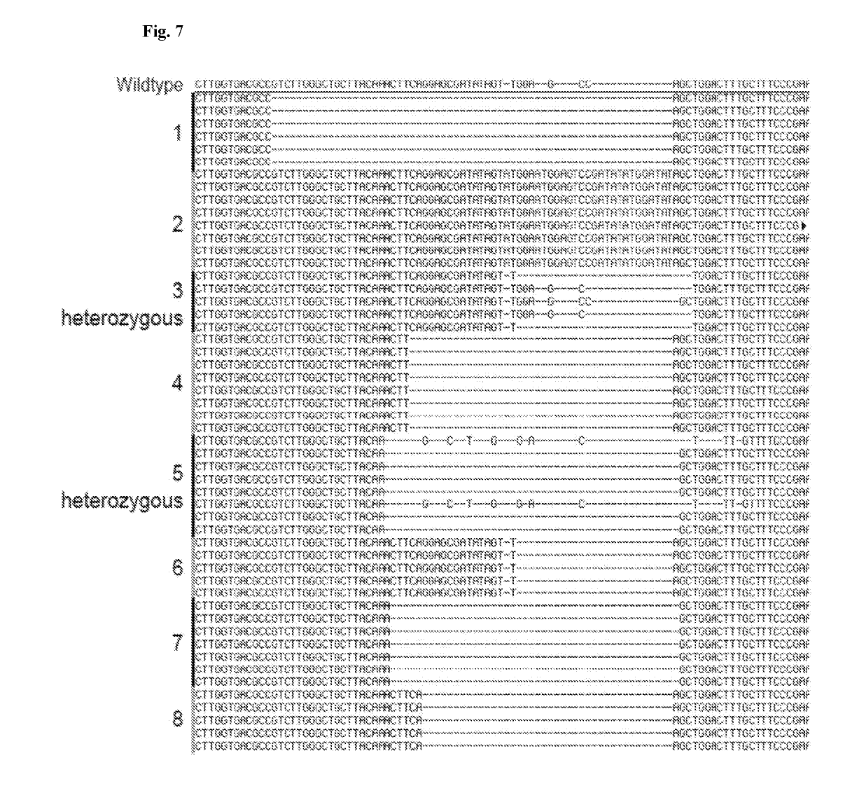

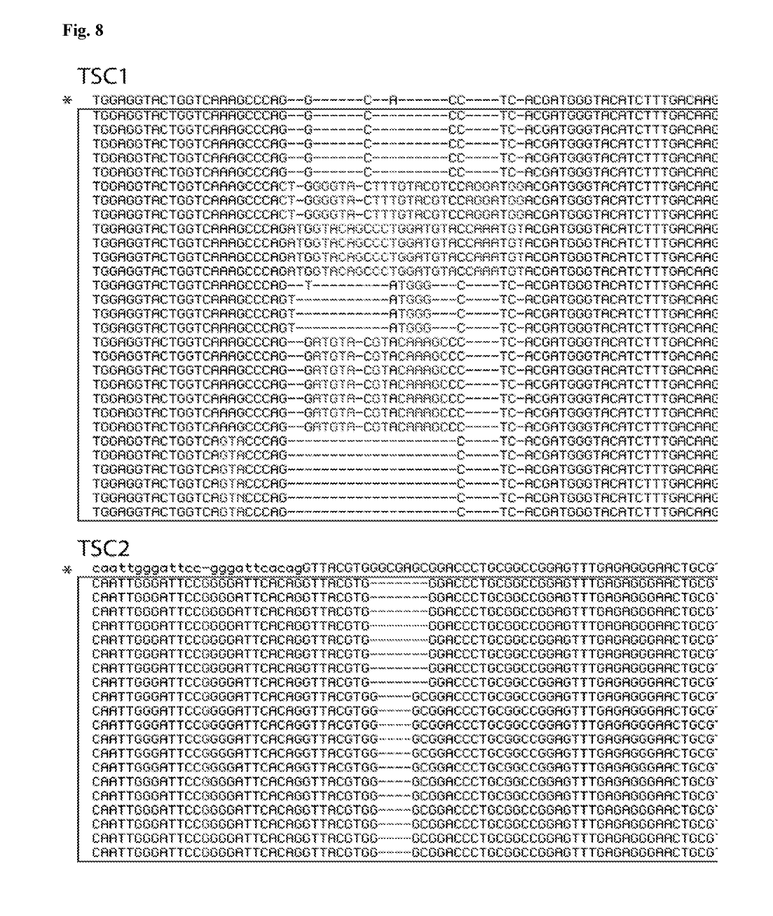

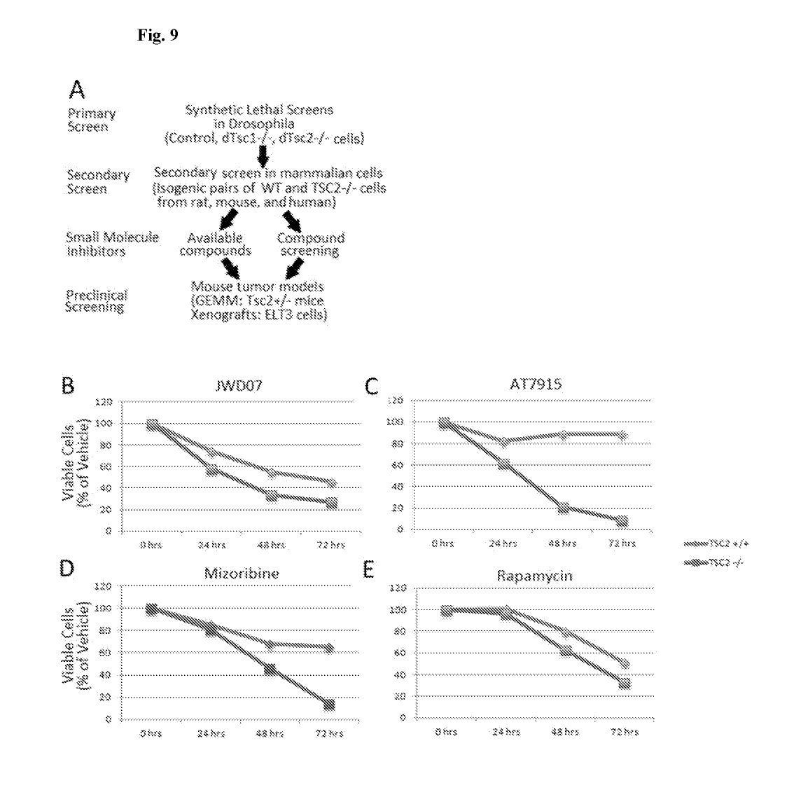

One difficulty associated with the isolation of mutant cells from Drosophila S2R+ cells is that they are aneuploid, containing roughly 4 copies of any given genomic locus (33). Thus, the chances of identifying cells in which all alleles carry frame shift mutations are considerably lower than for diploid cells. To assess the ability of CRISPR to produce homozygous mutations in these cells, the yellow gene was targeted, and 30 individual cells were tested for the presence of mutations using high-resolution melt (HRM) assays. 21 (70%) carried mutations at the target locus (FIG. 2, panel B). The 8 samples with the strongest signal in the HRM assays were analyzed by sequencing. No wild-type sequences were detected for any of these samples, and 6/8 contained a single mutation in all derived sequences (FIG. 7). Therefore, the HRM assay is an effective method to identify fully mutant clones.

Next, to test the efficacy of our sgRNA design tool and the combined CRISPR and single-cell cloning approach (FIG. 2, panel C), two genes were targeted, for which genes loss of protein function could easily be assayed: STAT92E and Ligase4 (FIG. 2, panel D). 15 and 4 clones were analyzed for STAT92E and Ligase4 mutants respectively and 13 and 2 of these carried mutations on all alleles. Further testing showed that the expected phenotypes were produced from these knockouts, with the STAT92E line unable to respond to JAK STAT pathway stimulation (FIG. 2, panel E) and the Ligase4 line showing an increase in homologous recombination rate (FIG. 2, panel F). These results demonstrate that our approach provides an efficient CRISPR-based method for the production of stable, homogenous mutant Drosophila cell lines.

### Example 4. Synthetic Screens Using TSC1 and TSC2 Mutant Lines

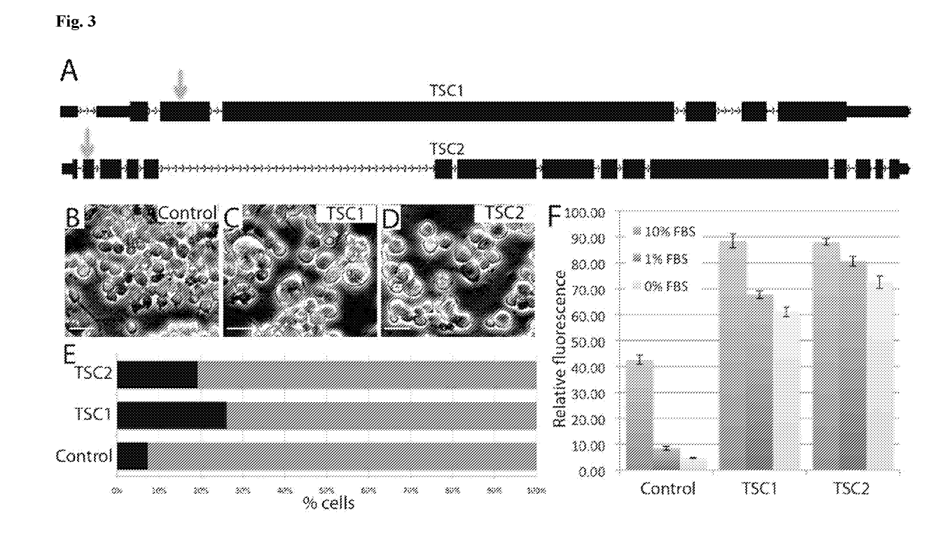

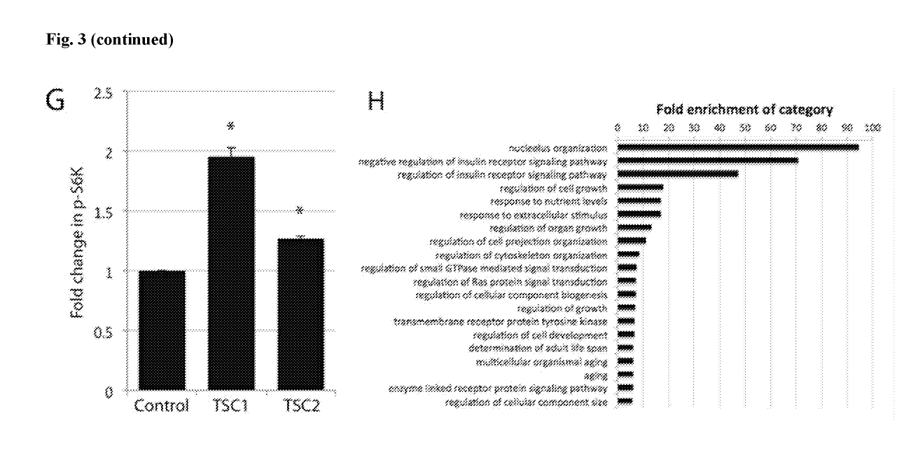

Cell lines were generated carrying frameshift mutations in the TSC1 and TSC2 genes using the approach described above (FIGS. 3 (panel A) and 8). To characterize the lines, as antibodies against Drosophila TSC1 or TSC2 are not available, the cell lines were tested for whether they showed phenotypes similar to those previously reported in vivo or in mammalian cell lines (34-38). Three phenotypes were considered: cell size, responsiveness to growth factor deprivation, and phosphorylation of the downstream Tor target S6k. All three phenotypes were present in TSC1 and TSC2 cell lines as they displayed an increased cell diameter, an inability to modify population growth in the absence of growth factors, and increased S6k phosphorylation levels (FIG. 3, panels B-G). To further characterize the mutant cell lines, a phosphoproteomic analysis was performed. 128 phosphosites showed greater than 1.5 fold increase or decrease in both mutant lines compared to wild-type cells (Table 3). GO analysis demonstrated that 20 of the top 30 most significantly enriched categories were consistent with known functions of the TSC network (FIG. 3, panel H and Table 4), including insulin signaling, response to nutrients, and the growth of cells and tissues. Together these results indicate that the cell lines accurately represent TSC mutant models.

TABLE 3

| | ANNOTATION | | Site | | Max | Redun- | |
|---|---|---|---|---|---|---|---|
| Protein Id | SYMBOL | SYMBOL | Position | Motif | Score | dancy | Sequence |
| FBpp0086897 | CG3821-PA | Aats-asp-PA | 48 | NNAGGDSAED | 162.876908 | R | K.AENASTAAANNAGGDS#AEDH |
| | | | | HAA | | | AAGR.Y |
| FBpp0080689 | CG10473-PA | Acn-PA | 319 | KKSSDRTPPVL | 25.1743944 | R | K.SSDRT#PPVLAISTDSLK.N |
| | | | | AI | | | |
| FBpp0079279 | CG13388-PA | Akap200-PA | 117 | TPLADESIKSKSK | 42.8691538 | R | K.EAAAGEDITPLADES#IKS#K.S |
| FBpp0081374 | CG9748-PA | bel-PA | 115 | RGFNRQSGDY | 57.7110635 | R | R.GFNRQS#GDYGYGSGGGGR.R |
| | | | | GYG | | | |
| FBpp0088026 | CG1363-PA | blow-PA | 495 | QRKSTTSPQSS | 23.6237902 | R | K.STTS#PQSS#PVSKDCDK.L |
| | | | | PV | | | |
| FBpp0087402 | CG12892-PA | Caf1-105-PA | 614 | SSQFNLTPKSQ | 39.6628888 | U | R.T#PGSSSQFNLT#PK.S |
| | | | | PA | | | |
| FBpp0085589 | CG11788-PA | CG11788-PA | 122 | STSPLKSPRTG | 26.1050926 | U | K.FAAAT#STSPLKS#PR.T |
| | | | | NA | | | |
| FBpp0081445 | CG11982-PA | CG11982-PA | 340 | TGTDNPSPAN | 64.7574047 | U | R.RSASTATGTDNPS#PANNPSQA |
| | | | | NPS | | | AAEGGR.T |
| FBpp0079497 | CG13124-PA | CG13124-PA | 260 | ALQRSKSLSSA | 15.0072508 | R | R.SKS#LSSADALTR.G |
| | | | | DA | | | |
| FBpp0077444 | CG15390-PA | CG15390-PA | 271 | KEEDELSDEDD | 1000 | U | R.RKEEDELS#DEDD.- |
| | | | | xx | | | |
| FBpp0070743 | CG15784-PA | CG15784-PA | 288 | HSLPRLSRRDC | 30.6121019 | R | R.SHSLPRLS#R.R |
| | | | | RG | | | |
| FBpp0070743 | CG15784-PA | CG15784-PA | 283 | ANRRSHSLPRL | 1000 | R | R.S#HS#LPR.L |
| | | | | SR | | | |
| FBpp0073924 | CG16952-PA | CG16952-PA | 996 | FMQRSNSPFEI | 17.0090955 | R | R.SNS#PFEILR.Q |
| | | | | LR | | | |
| FBpp0073367 | CG1703-PA | CG1703-PA | 47 | GGGGDSSDED | 65.4395866 | R | R.GGGGGDS#S#DEDVTTR.N |
| | | | | VTT | | | |
| FBpp0073309 | CG1737-PA | CG1737-PA | 724 | LVPQRRSKEN | 1000 | R | R.S#KENQQEQQR.D |
| | | | | QQE | | | |
| FBpp0087337 | CG30015-PA | CG30015-PA | 590 | RLIHRNSKRSIAR | 1000 | R | R.LIHRNS#KR.S |
| FBpp0083870 | CG31145-PA | CG31145-PA | 527 | GSETDVSSxxxxx | 37.5398384 | R | R.NPPGDVDGS#ETDVS#S.- |
| FBpp0111615 | CG34417-PC | CG34417-PC | 572 | YQEPQRSPQKL | 35.3557543 | R | R.TIETEVVGDDYQEPQRS#PQK.L |
| | | | | RE | | | |
| FBpp0111615 | CG34417-PC | CG34417-PC | 816 | SEPRSPTPKAG | 14.4607703 | R | K.TASEPRS#PT#PK.A |
| | | | | GS | | | |
| FBpp0291823 | CG42669-RL | CG42669-RL | 554 | LSNSELSPKSP | 15.381154 | R | R.RLSNS#ELS#PK.S |
| | | | | GA | | | |
| FBpp0291856 | CG42674-PE | CG42674-PE | 466 | HNVYRESLDSR | 16.9639219 | R | R.VHNVYRES#LDS#RTLDLLNQR.T |
| | | | | TL | | | |
| FBpp0291856 | CG42674-PE | CG42674-PE | 221 | TSPRKTSVADL | 17.0090955 | R | R.KTS#VADLQASS#PVLLR.R |
| | | | | QA | | | |
| FBpp0291856 | CG42674-PE | CG42674-PE | 421 | VMRRRRSKTSL | 17.0090955 | R | R.S#KTS#LEGR.S |
| | | | | EG | | | |
| FBpp0292725 | CG42797-PD | CG42797-PD | 448 | SPKRPRSPPSG | 23.6237902 | R | K.RPRS#PPSGCSTR.S |
| | | | | CS | | | |
| FBpp0084501 | CG6066-PA | CG6066-PA | 189 | PEVWGKSPSR | 22.0107646 | U | K.S#PSRPET#DDVELVK.G |
| | | | | PET | | | |
| FBpp0084501 | CG6066-PA | CG6066-PA | 64 | NRSSRRSREKD | 1000 | U | R.S#REKDAVPR.R |
| | | | | AV | | | |
| FBpp0083217 | CG6231-PC | CG6231-PC | 569 | NGKRKQSLIDA | 1000 | R | R.KQS#LIDADHEEQALKDANH.- |
| | | | | DH | | | |
| FBpp0083217 | CG6231-PC | CG6231-PC | 549 | KYKRKHSLIDA | 1000 | R | K.RKHS#LIDAK.Q |
| | | | | KQ | | | |
| FBpp0070639 | CG6379-PA | CG6379-PA | 788 | ADKLGHSxxxx | 1000 | R | K.FIADKLGHS#.- |
| | | | | xx | | | |
| FBpp0074372 | CG6540-PA | CG6540-PA | 308 | RKDNIISPQKD | 1000 | U | R.KDNIIS#PQKDVPQK.S |
| | | | | VP | | | |
| FBpp0072688 | CG7971-PA | CG7971-PA | 636 | AKEARPTRSPR | 20.9846301 | R | R.ERPAKEARPT#RSPR.E |
| | | | | ER | | | |
| FBpp0082995 | CG8064-PA | CG8064-PA | 321 | VAEDEGSDEA | 1000 | U | K.VAEDEGS#DEAK.L |
| | | | | KLS | | | |
| FBpp0087733 | CG8243-PA | CG8243-PA | 182 | SGDKRLSGSSA | 20.1669212 | U | K.RLS#GSSASALK.N |
| | | | | SA | | | |
| FBpp0073918 | CG8578-PA | CG8578-PA | 105 | SASRRSSEDSV | 24.436975 | R | R.RSS#EDS#VEEEGR.S |
| | | | | EE | | | |
| FBpp0076591 | CG8596-PA | CG8596-PA | 539 | ILGKHGSQHSI | 22.4917217 | R | K.HGS#QHS#ISHA.- |
| | | | | SH | | | |
| FBpp0079677 | CG5686-PA | chico-PA | 784 | KLVHSISSEDYTQ | 13.37953 | R | K.KLVHSIS#SEDYTQIKDK.S |
| FBpp0085066 | CG31012-PC | cindr-PC | 787 | RATRPNSLAIR | 36.556393 | R | R.ATRPNS#LAIR.N |
| | | | | NQ | | | |
| FBpp0085066 | CG31012-PC | cindr-PC | 494 | LDEKVKSPPPP | 86.8328304 | R | K.VKS#PPPPVLSK.K |
| | | | | VL | | | |
| FBpp0071165 | CG12737-PA | Crag-PA | 1080 | QSPTKISPRTP | 22.4917217 | R | R.VQSPT#KIS#PR.T |
| | | | | VT | | | |
| FBpp0074017 | CG9916-PA | Cyp1-PA | 226 | IVANSGSLxxxxx | 17.0090955 | U | K.IIVANSGS#L- |
| FBpp0081467 | CG9745-PC | D1-PC | 268 | KKRGRPSLAAG | 1000 | R | R.GRPS#LAAGK.V |
| | | | | KV | | | |
| FBpp0077759 | CG11371-PB | dbr-PB | 185 | NTGRRNSVFT | 13.37953 | R | R.RNS#VFTDTR.Y |
| | | | | DTR | | | |
| FBpp0288765 | CG1021-PF | Dmtn-PF | 105 | RGFRSHSPTHR | 15.0072508 | R | R.GFRS#HS#PTHR.R |
| | | | | RR | | | |
| FBpp0072041 | CG5602-PA | DNA-ligl-PA | 20 | DATDSPSPPKK | 18.8053684 | U | K.KSDATDS#PS#PPK.K |
| | | | | VP | | | |
| FBpp0113035 | CG5923-PC | DNApol- | 147 | KGAALFSPASY | 33.6571906 | U | K.GAALFS#PASYT#PQSAK.R |
| | | alpha73-PC | | TP | | | |
| FBpp0072804 | CG1044-PB | dos-PB | 774 | VPSSVVYRSVD | 37.5398384 | R | R.APVPSSVVY#R.S |
| | | | | FV | | | |
| FBpp0072804 | CG1044-PB | dos-PB | 680 | HRHHPNSPGS | 23.6237902 | R | R.HHPNS#PGSMSVQHQR.T |
| | | | | MSV | | | |
| FBpp0083178 | CG34157-PA | Dys-PA | 1866 | VEKRRISFDEK | 1000 | R | R.RIS#FDEKR.K |
| | | | | RK | | | |
| FBpp0088303 | CG10811-PA | eIF4G-PA | 346 | KKIPIVSPKNVSE | 1000 | R | K.KIPIVS#PK.N |
| FBpp0085768 | CG15112-PB | ena-PB | 133 | PKAGPIYEAPQ | 1000 | R | K.AGPIY#EAPQR.S |
| | | | | RS | | | |
| FBpp0073011 | CG10847-PB | enc-PB | 267 | QHNRTNSNGS | 28.9337487 | R | R.TNS#NGSGMEFNNNNNSSNKK.F |
| | | | | GME | | | |
| FBpp0302757 | CG14998-PK | ens-PK | 452 | SPSAQTTPKRT | 17.0090955 | R | K.TTAAS#PSAQTT#PKR.T |
| | | | | AN | | | |
| FBpp0302757 | CG14998-PK | ens-PK | 180 | ASLTRRSSERELA | 17.0090955 | R | R.S#SERELADSGAK.K |
| FBpp0302757 | CG14998-PK | ens-PK | 425 | IAGTGMSLEEI | 24.6317331 | R | R.KPRPASIAGTGMS#LEEINK.L |
| | | | | NK | | | |
| FBpp0086935 | CG8581-PA | fra-PA | 465 | RRKPFNSGEQL | 1000 | R | R.RKPFNS#GEQLR.T |
| | | | | RT | | | |
| FBpp0080391 | CG4274-PA | fzy-PA | 78 | TKKSNTTPSKT | 20.1669212 | U | K.SNTT#PSKT#PGGGDR.F |
| | | | | PG | | | |
| FBpp0072455 | CG12030-PA | Gale-PA | 304 | TLVDRRSGDV | 17.5124912 | R | R.S#GDVATCYADATLADKK.L |
| | | | | ATC | | | |
| FBpp0074588 | CG6975-PA | gig-PA | 1046 | DAAPPLSPERE | 55.3699546 | R | K.SLSDAAPPLS#PERER.R |
| | | | | RR | | | |
| FBpp0082494 | CG6904-PC | GlyS-PC | 10 | RFSRVESGADL | 1000 | R | R.VES#GADLK.D |
| | | | | KD | | | |
| FBpp0082494 | CG6904-PC | GlyS-PC | 651 | IFSRPHSEPPSPT | 27.1205177 | R | K.NNLIFSRPHS#EPPS#PTSSR.H |
| FBpp0071726 | CG5820-PA | Gp150-PA | 789 | DEKKADSAEAK | 1000 | R | K.KADS#AEAKLEK.R |
| | | | | LE | | | |
| FBpp0071726 | CG5820-PA | Gp150-PA | 932 | AEEVDMSQET | 16.0619943 | R | K.TDGIQAEEVDM*S#QETR.D |
| | | | | RDK | | | |
| FBpp0075463 | CG13475-PA | HGTX-PA | 131 | RYEHNSSPGV | 26.9265433 | U | R.YEHNS#S#PGVDSAK.S |
| | | | | DSA | | | |
| FBpp0078974 | CG10377-PA | Hrb27C-PA | 182 | GSGGQNSNNS | 30.6121019 | R | R.DGSGGQNS#NNSTVGGAYGK.L |
| | | | | TVG | | | |
| FBpp0076182 | CG4466-PA | Hsp27-PA | 75 | QMSRRASGGP | 1000 | U | R.RAS#GGPNALLPAVGK.D |
| | | | | NAL | | | |
| FBpp0076092 | CG6718-PB | iPLA2-VIA-PB | 526 | KENSSDSLASG | 31.2747123 | R | K.KENS#SDS#LASGSQK.S |
| | | | | SQ | | | |
| FBpp0081928 | CG31363-PD | Jupiter-PD | 35 | GSEMPQTPRN | 44.9729759 | R | K.VLRPPGGGSSDIFGSEMPQT#P |
| | | | | VKN | | | R.N |
| FBpp0073331 | CG1453-PA | Klp10A-PA | 210 | GASTRRSHALK | 1000 | R | R.S#HALKEVER.L |
| | | | | EV | | | |
| FBpp0289294 | CG12008-PE | kst-PE | 4266 | ESNRPVSLQPD | 22.4917217 | R | R.FESNRPVS#LQPDSISFSR.V |
| | | | | SI | | | |
| FBpp0289294 | CG12008-PE | kst-PE | 3762 | NVKRAESMKV | 1000 | R | K.RAES#MKVQPK.Q |
| | | | | QPK | | | |
| FBpp0071307 | CG32701-PA | l(1)G0320-PA | 272 | KSKTPKTSPKP | 14.9660036 | R | K.T#SPKPTKPAS#PK.A |
| | | | | TK | | | |
| FBpp0086068 | CG4798-PA | l(2)k01209-PA | 68 | AKASLDSPLKR | 15.0072508 | R | K.ASLDS#PLKR.S |
| | | | | SG | | | |
| FBpp0075723 | CG6801-PA | l(3)j2D3-PA | 104 | SDEGSPTGPQ | 14.0027653 | U | R.M*KSSLQALLSDEGS#PT#GPQ |
| | | | | RTA | | | R.T |
| FBpp0078733 | CG6944-PA | Lam-PA | 435 | RNSTRATPSRR | 29.6669198 | R | R.AT#PS#RRTPSAAVK.R |
| | | | | TP | | | |
| FBpp0078733 | CG6944-PA | Lam-PA | 591 | TSSSRLSRRRSVT | 13.37953 | R | R.IVSQHTSSSRLS#R.R |
| FBpp0081255 | CG2684-PA | lds-PA | 281 | RRSRIKSEDQK | 1000 | U | R.IKS#EDQK.V |
| | | | | VV | | | |
| FBpp0078561 | CG14648-PA | lost-PA | 344 | VNRRRRTTKSE | 14.4607703 | U | R.RT#T#KS#EGDQSGVEAGAK.T |
| | | | | GD | | | |
| FBpp0292610 | CG8709-PG | Lpin-PG | 337 | TPIQSDSELETTM | 19.8464547 | R | R.AAGGRPST#PIQSDS#ELETTMR |
| | | | | | | | DNR.H |
| FBpp0288789 | CG42250-PC | lqfR-PC | 275 | RSKTVSSPVSK | 17.0090955 | R | R.SKTVSS#PVSK.Q |
| | | | | QP | | | |
| FBpp0085234 | CG1483-PA | Map205-PA | 992 | PSFSTRSPNKQ | 26.1050926 | R | R.S#PNKQQSNGLGK.N |
| | | | | QS | | | |
| FBpp0078631 | CG3753-PA | Marcal1-PA | 63 | NQPQAKSPLN | 16.1440411 | U | K.S#PLNFYRS#PTGEQK.K |
| | | | | FYR | | | |
| FBpp0300534 | CG32156-PN | Mbs-PN | 862 | TPAALESPVRL | 62.6585232 | R | K.SSSAATTPAALES#PVRLR.D |
| | | | | RD | | | |
| FBpp0089229 | CG32464-PG | mtd-PG | 159 | PVERKLSGDES | 44.2778144 | R | R.KLS#GDESR.E |
| | | | | RE | | | |
| FBpp0089229 | CG32464-PG | mtd-PG | 426 | LSNSRRSSEDE | 137.135003 | R | R.RS#S#EDEGDNESNVTVDSGAR.V |
| | | | | GD | | | |
| FBpp0089229 | CG32464-PG | mtd-PG | 41 | RRSKSRSVDHG | 145.27528 | R | R.S#VDHGLAAPFDLDSLR.S |
| | | | | LA | | | |
| FBpp0089229 | CG32464-PG | mtd-PG | 17 | YWSRRASQDV | 44.2778144 | R | R.RAS#QDVSSLSR.S |
| | | | | SSL | | | |
| FBpp0089229 | CG32464-PG | mtd-PG | 26 | VSSLSRSVDNL | 1000 | R | R.S#VDNLAIPVRR.S |
| | | | | AI | | | |
| FBpp0089229 | CG32464-PG | mtd-PG | 176 | EGLRPGSPKPG | 1000 | R | K.DILEGLRPGS#PKPGHIER.V |
| | | | | HI | | | |
| FBpp0089229 | CG32464-PG | mtd-PG | 65 | VEQRFESVDKL | 51.5723038 | R | R.FES#VDKLSR.Q |
| | | | | SR | | | |
| FBpp0305169 | CG17255-PD | nocte-PD | 1180 | ANKAGFSPRG | 1000 | R | K.AGFS#PR.G |
| | | | | EPS | | | |
| FBpp0305169 | CG17255-PD | nocte-PD | 1036 | TQILRRSVDIDKS | 1000 | R | R.S#VDIDK.S |
| FBpp0089003 | CG7421-PA | Nopp140-PA | 704 | GGFGNKSFDSS | 53.5336889 | R | K.S#FDSSAPK.Q |
| | | | | AP | | | |
| FBpp0073671 | CG10990-PA | Pdcd4-PA | 337 | ALRRADSLIYK | 45.0259609 | R | R.RADS#LIYK.H |
| | | | | HV | | | |
| FBpp0074963 | CG6143-PB | Pep-PB | 703 | AAGAKATPQR | 1000 | R | R.AAAGAKAT#PQR.Q |
| | | | | QRA | | | |
| FBpp0070385 | CG2845-PA | phl-PA | 402 | NRPRARSADES | 34.0887043 | R | R.ARS#ADESNKNLLLR.D |
| | | | | NK | | | |
| FBpp0084559 | CG5692-PA | pins-PA | 633 | MEDQRASIPFR | 1000 | U | R.M*EDQRAS#IPFR.N |
| | | | | NA | | | |
| FBpp0300548 | CG4532-PG | pod1-PG | 767 | IDTKRISVPEGKL | 1000 | R | K.RIS#VPEGK.L |
| FBpp0086727 | CG30483-PA | Prosap-PA | 1180 | SANAKIYASPQ | 48.87395 | R | K.IY#ASPQELR.N |
| | | | | EL | | | |
| FBpp0086727 | CG30483-PA | Prosap-PA | 1182 | NAKIYASPQEL | 17.0090955 | R | K.IYAS#PQELR.N |
| | | | | RN | | | |
| FBpp0077992 | CG7752-PA | pzg-PA | 575 | HSDKNESGEA | 16.6191736 | R | K.LHSDKNES#GEAPKPGSK.G |
| | | | | PKP | | | |
| FBpp0305180 | CG4320-PB | raptor-PB | 890 | LTSSTHSLERH | 44.2778144 | U | R.DLTSSTHS#LER.H |
| | | | | VT | | | |
| FBpp0292404 | CG3585-PB | Rbcn-3A-PB | 1571 | RLKNVASMPLI | 38.1887792 | U | K.NVAS#MPLISK.V |
| | | | | SK | | | |
| FBpp0293740 | CG6831-PB | rhea-PB | 2822 | FTTTTESRSETKT | 20.9846301 | R | K.SFTTTTES#R.S |
| FBpp0110289 | CG4937-PB | RhoGAP15B- | 22 | ELRPANYENIEIR | 13.9355156 | R | R.RLYPELRPANY#ENIEIR.R |
| | | PB | | | | | |
| FBpp0288898 | CG42274-PC | RhoGAP18B- | 236 | QSDQHIYGRV | 71.9492873 | U | K.GSQSDQHIY#GR.V |
| | | PC | | REP | | | |
| FBpp0083230 | CG4755-PA | RhoGAP92B- | 593 | GLDNLPSPTAD | 20.1669212 | R | R.TQFFGLDNLPS#PTADRK.S |
| | | PA | | RK | | | |
| FBpp0086124 | CG9635-PF | RhoGEF2-PF | 1997 | RTMSIRSTGEPIQ | 24.436975 | R | R.S#TGEPIQK.Y |
| FBpp0086441 | CG30085-PA | Rif1-PA | 1067 | FSKRLPSPSASPS | 31.2747123 | U | K.RLPS#PSAS#PSVSILK.R |
| FBpp0074387 | CG6606-PA | Rip11-PA | 744 | SHADRRSEISA | 16.9639219 | R | K.SGSHADRRS#EISAQLAK.K |
| | | | | QL | | | |
| FBpp0081544 | CG16788-PA | RnpS1-PA | 114 | KDKDKPSARSR | 1000 | U | K.DKDKDKPS#AR.S |
| | | | | SR | | | |
| FBpp0293388 | CG18076-PY | shot-PY | 4965 | ITPTRDTPDRD | 15.0072508 | R | R.IT#PTRDT#PDRDR.L |
| | | | | RL | | | |
| FBpp0293388 | CG18076-PY | shot-PY | 2259 | RKRSTYSPIKRTS | 17.0090955 | R | R.STYS#PIKRT#SPLR.R |
| FBpp0081709 | CG12819-PB | sle-PB | 824 | LNELNKSERFN | 1000 | R | R.QVALNELNKS#ER.F |
| | | | | KT | | | |
| FBpp0077781 | CG18497-PA | spen-PA | 3152 | EKPRLISPIPKTP | 1000 | R | R.LIS#PIPK.T |
| FBpp0075617 | CG11274-PA | SRm160-PA | 617 | HSRKRESPIGR | 1000 | U | R.KRES#PIGR.S |
| | | | | SS | | | |
| FBpp0078827 | CG31641-PC | stai-PC | 67 | AEERRISLEAKKM | 1000 | R | R.RIS#LEAK.K |
| FBpp0079251 | CG8409-PA | Su(var)205-PA | 102 | ETQGRASSSTS | 17.0090955 | R | K.ETQGRAS#SSTSTASK.R |
| | | | | TA | | | |
| FBpp0072188 | CG4057-PA | tamo-PA | 397 | KTGPAHSCDL | 39.6628888 | R | K.TGPAHS#CDLHR.R |
| | | | | HRR | | | |
| FBpp0077213 | CG8846-PA | Thor-PA | 65 | MKNLRGSPLS | 71.7529246 | U | K.NLRGS#PLSQT#PPSNVPSCLLR.G |
| | | | | QTP | | | |
| FBpp0083931 | CG6147-PA | Tsc1-PA | 566 | PMRRTKSCSAL | 20.1669212 | U | R.TKS#CSALSGM*R.Q |
| | | | | SG | | | |
| FBpp0099885 | CG4347-PC | UGP-PC | 18 | GHQRAPSDSK | 23.6237902 | R | R.APS#DSKEFHEVTKR.D |
| | | | | EFH | | | |
| FBpp0081570 | CG31352-PA | Unc-115a-PA | 451 | ETPRPKSPGM | 117.022326 | R | K.S#PGM*NNEEPIELSHYPAAK.K |
| | | | | NNE | | | |
| FBpp0079999 | CG3762-PA | Vha68-2-PA | 130 | SLSRVASWEFN | 1000 | R | R.VAS#WEFNPLNVK.V |
| | | | | PL | | | |
| FBpp0084150 | CG11844-PB | vig2-PB | 426 | FGEKRRSAQKP | 46.0627025 | R | R.S#AQKPLKVDDEAQFPTLC- |
| | | | | LK | | | |
| FBpp0085375 | CG8390-PA | vlc-PA | 322 | AQLPTVSPKRG | 44.2778144 | R | R.AQLPTVS#PK.R |
| | | | | AP | | | |
| FBpp0072305 | CG15792-PB | zip-PB | 2009 | SLDGEDSANxx | 41.3022219 | R | K.RAGGGGGGDDSSVQDESLDGE |
| | | | | xx | | | DS#AN.- |
| FBpp0072305 | CG15792-PB | zip-PB | 1976 | GGIGLSSSRLT | 24.436975 | R | R.TGGIGLSS#SR.L |
| | | | | GT | | | |
| FBpp0072305 | CG15792-PB | zip-PB | 2003 | SSVQDESLDGE | 30.6121019 | R | K.RAGGGGGGDDSSVQDES#LDG |
| | | | | DS | | | EDS#AN.- |

TABLE 4

| | | | Fold | | | |
|---|---|---|---|---|---|---|
| Term | Term name | PValue | Enrichment | Count | % | Genes |
| GO: 0007000 | nucleolus | 0.02064757 | 94.4880952 | 2 | 2.27272727 | FBGN0037810, FBGN0037137 |
| | organization | | | | | |
| GO: 0046627 | negative regulation | 0.02743663 | 70.8660714 | 2 | 2.27272727 | FBGN0026317, FBGN0005198 |
| | of insulin receptor | | | | | |
| | signaling pathway | | | | | |
| GO: 0046626 | regulation of insulin | 0.04087641 | 47.2440476 | 2 | 2.27272727 | FBGN0026317, FBGN0005198 |
| | receptor signaling | | | | | |
| | pathway | | | | | |
| GO: 0001558 | regulation of cell | 0.00135472 | 17.7165179 | 4 | 4.54545455 | FBGN0026317, FBGN0005198, |
| | growth | | | | | FBGN0013733, FBGN0024248 |
| GO: 0031667 | response to | 0.01277241 | 17.0078571 | 3 | 3.40909091 | FBGN0026317, FBGN0005198, |
| | nutrient levels | | | | | FBGN0024248 |
| GO: 0009991 | response to | 0.01277241 | 17.0078571 | 3 | 3.40909091 | FBGN0026317, FBGN0005198, |
| | extracellular | | | | | FBGN0024248 |
| | stimulus | | | | | |
| GO: 0046620 | regulation of organ | 0.02047546 | 13.2873884 | 3 | 3.40909091 | FBGN0026317, FBGN0005198, |
| | growth | | | | | FBGN0024248 |
| GO: 0031344 | regulation of cell | 0.02826563 | 11.1893797 | 3 | 3.40909091 | FBGN0000578, FBGN0023172, |
| | projection | | | | | FBGN0013733 |
| | organization | | | | | |
| GO: 0006997 | nucleus | 0.02966312 | 10.9024725 | 3 | 3.40909091 | FBGN0037810, FBGN0002525, |
| | organization | | | | | FBGN0037137 |
| GO: 0060446 | branching involved | 0.02966312 | 10.9024725 | 3 | 3.40909091 | FBGN0002525, FBGN0003079, |
| | in open tracheal | | | | | FBGN0013733 |
| | system | | | | | |
| | development | | | | | |
| GO: 0048754 | branching | 0.02966312 | 10.9024725 | 3 | 3.40909091 | FBGN0002525, FBGN0003079, |
| | morphogenesis of a | | | | | FBGN0013733 |
| | tube | | | | | |
| GO: 0045664 | regulation of | 0.00608491 | 10.4986773 | 4 | 4.54545455 | FBGN0003079, FBGN0023172, |
| | neuron | | | | | FBGN0013733, FBGN0005536 |
| | differentiation | | | | | |
| GO: 0060562 | epithelial tube | 0.03401677 | 10.1237245 | 3 | 3.40909091 | FBGN0002525, FBGN0003079, |
| | morphogenesis | | | | | FBGN0013733 |
| GO: 0001763 | morphogenesis of a | 0.03401677 | 10.1237245 | 3 | 3.40909091 | FBGN0002525, FBGN0003079, |
| | branching structure | | | | | FBGN0013733 |
| GO: 0051493 | regulation of | 0.04673533 | 8.50392857 | 3 | 3.40909091 | FBGN0000578, FBGN0051363, |
| | cytoskeleton | | | | | FBGN0013733 |
| | organization | | | | | |
| GO: 0050767 | regulation of | 0.01103262 | 8.46162047 | 4 | 4.54545455 | FBGN0003079, FBGN0023172, |
| | neurogenesis | | | | | FBGN0013733, FBGN0005536 |
| GO: 0060341 | regulation of | 0.01148598 | 8.33718487 | 4 | 4.54545455 | FBGN0260003, FBGN0004875, |
| | cellular localization | | | | | FBGN0004838, FBGN0041582 |
| GO: 0051056 | regulation of small | 0.00396741 | 7.53894377 | 5 | 5.68181818 | FBGN0033349, FBGN0027932, |
| | GTPase mediated | | | | | FBGN0016794, FBGN0005198, |
| | signal transduction | | | | | FBGN0023172 |
| GO: 0046578 | regulation of Ras | 0.0171838 | 7.17631103 | 4 | 4.54545455 | FBGN0033349, FBGN0027932, |
| | protein signal | | | | | FBGN0016794, FBGN0023172 |
| | transduction | | | | | |
| GO: 0044087 | regulation of | 0.01776719 | 7.08660714 | 4 | 4.54545455 | FBGN0000578, FBGN0004875, |
| | cellular component | | | | | FBGN0004838, FBGN0051363 |
| | biogenesis | | | | | |
| GO: 0040008 | regulation of | 0.0054943 | 6.88020111 | 5 | 5.68181818 | FBGN0026317, FBGN0003079, |
| | growth | | | | | FBGN0005198, FBGN0013733, |
| | | | | | | FBGN0024248 |
| GO: 0007169 | transmembrane | 0.00670551 | 6.50147444 | 5 | 5.68181818 | FBGN0016977, FBGN0026317, |
| | receptor protein | | | | | FBGN0016794, FBGN0003079, |
| | tyrosine kinase | | | | | FBGN0024248 |
| | signaling pathway | | | | | |
| GO: 0060284 | regulation of cell | 0.00203821 | 6.49154853 | 6 | 6.81818182 | FBGN0004875, FBGN0004838, |
| | development | | | | | FBGN0003079, FBGN0023172, |
| | | | | | | FBGN0013733, FBGN0005536 |
| GO: 0051960 | regulation of | 0.02420793 | 6.29920635 | 4 | 4.54545455 | FBGN0003079, FBGN0023172, |
| | nervous system | | | | | FBGN0013733, FBGN0005536 |
| | development | | | | | |
| GO: 0008340 | determination of | 0.02635583 | 6.09600614 | 4 | 4.54545455 | FBGN0001226, FBGN0026317, |
| | adult life span | | | | | FBGN0005198, FBGN0024248 |
| GO: 0010259 | multicellular | 0.02635583 | 6.09600614 | 4 | 4.54545455 | FBGN0001226, FBGN0026317, |
| | organismal aging | | | | | FBGN0005198, FBGN0024248 |
| GO: 0007568 | aging | 0.02635583 | 6.09600614 | 4 | 4.54545455 | FBGN0001226, FBGN0026317, |
| | | | | | | FBGN0005198, FBGN0024248 |
| GO: 0007167 | enzyme linked | 0.00317126 | 5.86477833 | 6 | 6.81818182 | FBGN0016977, FBGN0026317, |
| | receptor protein | | | | | FBGN0016794, FBGN0003079, |
| | signaling pathway | | | | | FBGN0013272, FBGN0024248 |
| GO: 0032535 | regulation of | 0.03094978 | 5.72655123 | 4 | 4.54545455 | FBGN0000578, FBGN0026317, |
| | cellular component | | | | | FBGN0005198, FBGN0024248 |
| | size | | | | | |
| GO: 0007424 | open tracheal | 0.00399306 | 5.55812325 | 6 | 6.81818182 | FBGN0002525, FBGN0003079, |
| | system | | | | | FBGN0023458, FBGN0023172, |
| | development | | | | | FBGN0013733, FBGN0024248 |
| GO: 0060541 | respiratory system | 0.00399306 | 5.55812325 | 6 | 6.81818182 | FBGN0002525, FBGN0003079, |
| | development | | | | | FBGN0023458, FBGN0023172, |
| | | | | | | FBGN0013733, FBGN0024248 |
| GO: 0033043 | regulation of | 0.03948267 | 5.20117955 | 4 | 4.54545455 | FBGN0000578, FBGN0051363, |
| | organelle | | | | | FBGN0013733, FBGN0003607 |
| | organization | | | | | |
| GO: 0006325 | chromatin | 0.02972607 | 4.16859244 | 5 | 5.68181818 | FBGN0046214, FBGN0259785, |
| | organization | | | | | FBGN0033526, FBGN0003607, |
| | | | | | | FBGN0031655 |
| GO: 0002009 | morphogenesis of | 0.01928802 | 3.77952381 | 6 | 6.81818182 | FBGN0000578, FBGN0002525, |
| | an epithelium | | | | | FBGN0003079, FBGN0023172, |
| | | | | | | FBGN0013733, FBGN0005536 |
| GO: 0060429 | epithelium | 0.02280533 | 3.61869301 | 6 | 6.81818182 | FBGN0000578, FBGN0002525, |
| | development | | | | | FBGN0003079, FBGN0023172, |
| | | | | | | FBGN0013733, FBGN0005536 |
| GO: 0051276 | chromosome | 0.01283276 | 3.51817376 | 7 | 7.95454546 | FBGN0046214, FBGN0004875, |
| | organization | | | | | FBGN0259785, FBGN0030268, |
| | | | | | | FBGN0033526, FBGN0003607, |
| | | | | | | FBGN0031655 |
| GO: 0048729 | tissue | 0.02754925 | 3.44288606 | 6 | 6.81818182 | FBGN0000578, FBGN0002525, |
| | morphogenesis | | | | | FBGN0003079, FBGN0023172, |
| | | | | | | FBGN0013733, FBGN0005536 |
| GO: 0035220 | wing disc | 0.04426463 | 3.0263091 | 6 | 6.81818182 | FBGN0260003, FBGN0016977, |
| | development | | | | | FBGN0016794, FBGN0003079, |
| | | | | | | FBGN0013733, FBGN0005536 |
| GO: 0048707 | instar larval or | 0.03195886 | 2.85914986 | 7 | 7.95454546 | FBGN0260003, FBGN0016977, |
| | pupal | | | | | FBGN0027932, FBGN0003079, |
| | morphogenesis | | | | | FBGN0013733, FBGN0004432, |
| | | | | | | FBGN0005536 |
| GO: 0009886 | post-embryonic | 0.0343479 | 2.81055241 | 7 | 7.95454546 | FBGN0260003, FBGN0016977, |
| | morphogenesis | | | | | FBGN0027932, FBGN0003079, |
| | | | | | | FBGN0013733, FBGN0004432, |
| | | | | | | FBGN0005536 |
| GO: 0007552 | metamorphosis | 0.03814612 | 2.7406768 | 7 | 7.95454546 | FBGN0260003, FBGN0016977, |
| | | | | | | FBGN0027932, FBGN0003079, |
| | | | | | | FBGN0013733, FBGN0004432, |
| | | | | | | FBGN0005536 |
| GO: 0007010 | cytoskeleton | 0.0402784 | 2.43840246 | 8 | 9.09090909 | FBGN0260442, FBGN0000578, |
| | organization | | | | | FBGN0030268, FBGN0051352, |
| | | | | | | FBGN0023172, FBGN0023213, |
| | | | | | | FBGN0013733, FBGN0040080 |

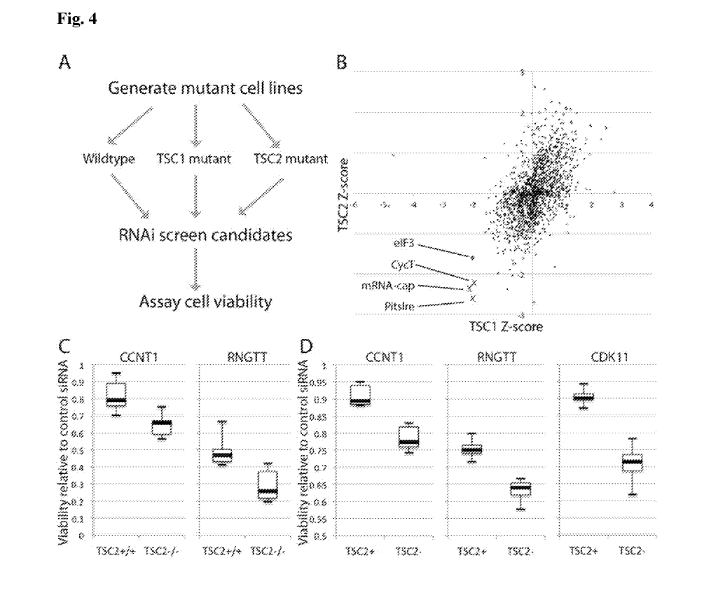

Next, taking advantage of the homogenous TSC1 and TSC2 mutant cell lines, a combinatorial RNAi screen of all Drosophila kinases (376) and phosphatases (159) was performed (FIG. 4, panel A). Any samples with significant effects on the viability of wild-type cells were discarded to identify TSC specific hits. 20 of the remaining knockdowns had significant effects on viability of TSC1 mutant cells and 49 hits significantly affected TSC2 mutant cells (FIG. 4, panel B and Table 5). GO analysis of these hits showed significant enrichment of several categories related to cell proliferation and growth (22 categories), metabolic processes (7 categories), and cytoskeletal regulation and cell shape (7 categories), processes known to be regulated by the TSC signaling network. As TSC1 and TSC2 act as a heterodimer and mutations in either gene give rise to the TSC disease, further studies were performed to identify hits that caused reduced viability in both the TSC1 and TSC2 screens. Using this approach, the noise associated with either individual screen is filtered to identify genes with the most robust synthetic interactions with the TSC complex. The knockdown of three genes (mRNA-cap/RNGTT, Pitslre/CDK11 and CycT/CCNT1) showed robust and specific effects on TSC1 and TSC2 deficient cell viability (FIG. 4, panel B, purple crosses).

mRNA-cap/RNGTT is a phosphatase required for the addition of a 5′ 7-methylguanylate cap to mRNAs, which is necessary for the initiation of cap-dependent translation. As activation of mTOR promotes cap-dependent translation initiation through multiple downstream targets (39, 40), these findings indicate that survival of TSC mutant cells is dependent on mRNA capping, an event preceding the steps in translation regulated by mTOR. Interestingly, this phosphoproteomic analysis identified phophosites on distinct components of the translation initiation machinery, such as Thor/4E-BP, eIF4G, eIF3-S10 and eIF2B, being either up or down in both TSC mutant cell lines compared to control. In addition, phosphorylation changes were detected in both cell lines for three other proteins that directly interact with core components of the translation initiation complex (Ens/MAP7, Map205/MUC16, Shot/DST) (41, 42).

Given the close link between TSC signaling and translation initiation, tests were performed to whether another translation initiation component regulated downstream of TSC showed synthetic viability decrease in the TSC mutant cell lines. eIF3 was knocked down in wild-type or TSC mutant S2R+ cells using the same assays as for the kinase/phosphatase screen. A synthetic viability decrease was observed in both TSC1 and TSC2 mutant cells for all three of these knockdowns (FIG. 4, panel B, purple circle) suggesting that the control of cap-dependent translation initiation may be a promising therapeutic target for TSC dependent disease.

CycT/CCNT1 is a kinase implicated in regulation of mitosis and transcriptional elongation (43, 44) with no known link to TSC signaling. Pistlre/CDK11 is a cyclin dependent kinase that has been implicated in the regulation of autophagy (45).

### Example 5. Synthetic Interactions are Conserved in Mammalian Cells

As all three hits from the Drosophila screens have clear orthologs in mammals (Table 6), experiments were performed to test whether the synthetic lethal interactions between mRNA-cap/RNGTT, Pitslre/CDK11 and CycT/CCNT1 and TSC1 and TSC2 are conserved. siRNAs were used, which target homologs of each of the three genes in TSC2-deficient MEFs compared to littermate-derived wild-type MEFs. Both RNGTT and CCNT1 knockdowns caused significantly reduced growth rate in TSC2 cells compared to wild type (p<0.05) (FIG. 4, panel C). Further, to assess the relevance of these potential drug targets to human tumor cells, siRNA was used to knockdown the three hits in a TSC2-deficient human cell line derived from a renal angiomyolipoma (AML) from a LAM patient (47). For isogenic comparison, the candidate genes were knocked down using siRNA in the same cell line reconstituted with wild-type TSC2. siRNAs targeting each of these three genes demonstrated significant selective inhibition of growth in the TSC2 null cells (p<0.05) (FIG. 4, panel D), indicating that these gene products are promising drug targets for TSC and LAM.

TABLE 6

| | | | | | | | | | Prediction |
|---|---|---|---|---|---|---|---|---|---|
| Search | Fly | | Fly | Human | | Human | DIOPT | Weighted | Derived |
| Term | GeneID | FlyBaseID | Symbol | GeneID | HGNCID | Symbol | Score | Score | From |
| CG10637 | 35154 | FBgn0015772 | Nak | 22848 | 19679 | AAK1 | 8 | 7.659 | Compara, |
| | | | | | | | | | Inparanoid, |
| | | | | | | | | | Isobase, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG10637 | 35154 | FBgn0015772 | Nak | 55589 | 18041 | BMP2K | 5 | 4.744 | Compara, |
| | | | | | | | | | OrthoDB, |
| | | | | | | | | | orthoMCL, |
| | | | | | | | | | Phylome, |
| | | | | | | | | | RoundUp |
| CG1810 | 32379 | FBgn0030556 | mRNA-cap | 8732 | 10073 | RNGTT | 10 | 9.669 | Compara, |
| | | | | | | | | | Homologene, |
| | | | | | | | | | Inparanoid, |
| | | | | | | | | | Isobase, |
| | | | | | | | | | OMA, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG1810 | 32379 | FBgn0030556 | mRNA-cap | 54935 | 21480 | DUSP23 | 1 | 0.91 | Phylome |
| CG2577 | 32188 | FBgn0030384 | CG2577 | 122011 | 20289 | CSNK1A1L | 1 | 0.96 | TreeFam |
| CG2577 | 32188 | FBgn0030384 | CG2577 | 1452 | 2451 | CSNK1A1 | 1 | 0.96 | TreeFam |
| CG2577 | 32188 | FBgn0030384 | CG2577 | 1455 | 2455 | CSNK1G2 | 1 | 0.93 | Compara |
| CG2577 | 32188 | FBgn0030384 | CG2577 | 53944 | 2454 | CSNK1G1 | 1 | 0.93 | Compara |
| CG2577 | 32188 | FBgn0030384 | CG2577 | 1456 | 2456 | CSNK1G3 | 1 | 0.93 | Compara |
| CG3051 | 43904 | FBgn0023169 | SNF1A | 5563 | 9377 | PRKAA2 | 10 | 9.669 | Compara, |
| | | | | | | | | | Homologene, |
| | | | | | | | | | Inparanoid, |
| | | | | | | | | | Isobase, |
| | | | | | | | | | OMA, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG3051 | 43904 | FBgn0023169 | SNF1A | 5562 | 9376 | PRKAA1 | 8 | 7.719 | Compara, |
| | | | | | | | | | Inparanoid, |
| | | | | | | | | | OMA, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG3051 | 43904 | FBgn0023169 | SNF1A | 9024 | 11405 | BRSK2 | 1 | 0.9 | orthoMCL |
| CG3051 | 43904 | FBgn0023169 | SNF1A | 84446 | 18994 | BRSK1 | 1 | 0.9 | orthoMCL |
| CG4268 | 40292 | FBgn0016696 | Pitslre | 984 | 1729 | CDK11B | 8 | 7.699 | Compara, |
| | | | | | | | | | Homologene, |
| | | | | | | | | | Inparanoid, |
| | | | | | | | | | Isobase, |
| | | | | | | | | | OrthoDB, |
| | | | | | | | | | orthoMCL, |
| | | | | | | | | | Phylome, |
| | | | | | | | | | RoundUp |
| CG4268 | 40292 | FBgn0016696 | Pitslre | 728642 | 1730 | CDK11A | 6 | 5.828 | Homologene, |
| | | | | | | | | | Inparanoid, |
| | | | | | | | | | Isobase, |
| | | | | | | | | | Phylome, |
| | | | | | | | | | RoundUp, |
| | | | | | | | | | TreeFam |
| CG4268 | 40292 | FBgn0016696 | Pitslre | 8558 | 1770 | CDK10 | 2 | 2.004 | OrthoDB, |
| | | | | | | | | | RoundUp |
| CG4317 | 31544 | FBgn0026060 | Mipp2 | 9562 | 7102 | MINPP1 | 9 | 8.668 | Compara, |
| | | | | | | | | | Homologene, |
| | | | | | | | | | Inparanoid, |
| | | | | | | | | | Isobase, |
| | | | | | | | | | OMA, |
| | | | | | | | | | orthoMCL, |
| | | | | | | | | | Phylome, |
| | | | | | | | | | RoundUp, |
| | | | | | | | | | TreeFam |
| CG44153 | 34341 | FBgn0265002 | CG44153 | 575 | 943 | BAI1 | 1 | 0.91 | Phylome |
| CG44153 | 34341 | FBgn0265002 | CG44153 | 577 | 945 | BAI3 | 1 | 0.91 | Phylome |
| CG5072 | 36854 | FBgn0016131 | Cdk4 | 1021 | 1777 | CDK6 | 9 | 8.666 | Compara, |
| | | | | | | | | | Homologene, |
| | | | | | | | | | Inparanoid, |
| | | | | | | | | | Isobase, |
| | | | | | | | | | OMA, |
| | | | | | | | | | OrthoDB, |
| | | | | | | | | | orthoMCL, |
| | | | | | | | | | Phylome, |
| | | | | | | | | | TreeFam |
| CG5072 | 36854 | FBgn0016131 | Cdk4 | 1019 | 1773 | CDK4 | 7 | 6.819 | Compara, |
| | | | | | | | | | Inparanoid, |
| | | | | | | | | | OMA, |
| | | | | | | | | | OrthoDB, |
| | | | | | | | | | Phylome, |
| | | | | | | | | | RoundUp, |
| | | | | | | | | | TreeFam |
| CG5072 | 36854 | FBgn0016131 | Cdk4 | 2268 | 3697 | FGR | 1 | 0.91 | Phylome |
| CG5387 | 34385 | FBgn0027491 | Cdk5alpha | 8851 | 1775 | CDK5R1 | 7 | 6.654 | Compara, |
| | | | | | | | | | Isobase, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG5387 | 34385 | FBgn0027491 | Cdk5alpha | 8941 | 1776 | CDK5R2 | 6 | 5.756 | Compara, |
| | | | | | | | | | Inparanoid, |
| | | | | | | | | | Isobase, |
| | | | | | | | | | OrthoDB, |
| | | | | | | | | | Phylome, |
| | | | | | | | | | TreeFam |
| CG5650 | 49260 | FBgn0004103 | Pp1-87B | 5501 | 9283 | PPP1CC | 9 | 8.719 | Compara, |
| | | | | | | | | | Homologene, |
| | | | | | | | | | Inparanoid, |
| | | | | | | | | | OMA, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG5650 | 49260 | FBgn0004103 | Pp1-87B | 5499 | 9281 | PPP1CA | 9 | 8.669 | Compara, |
| | | | | | | | | | Inparanoid, |
| | | | | | | | | | Isobase, |
| | | | | | | | | | OMA, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG5650 | 49260 | FBgn0004103 | Pp1-87B | 5500 | 9282 | PPP1CB | 1 | 0.9 | orthoMCL |
| CG6292 | 39961 | FBgn0025455 | CycT | 904 | 1599 | CCNT1 | 9 | 8.659 | Compara, |
| | | | | | | | | | Homologene, |
| | | | | | | | | | Inparanoid, |
| | | | | | | | | | Isobase, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG6292 | 39961 | FBgn0025455 | CycT | 905 | 1600 | CCNT2 | 7 | 6.709 | Compara, |
| | | | | | | | | | Inparanoid, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG6292 | 39961 | FBgn0025455 | CycT | 8812 | 1596 | CCNK | 1 | 1.001 | OrthoDB |
| CG9096 | 32551 | FBgn0010315 | CycD | 894 | 1583 | CCND2 | 7 | 6.708 | Compara, |
| | | | | | | | | | Homologene, |
| | | | | | | | | | Inparanoid, |
| | | | | | | | | | orthoMCL, |
| | | | | | | | | | Phylome, |
| | | | | | | | | | RoundUp, |
| | | | | | | | | | TreeFam |
| CG9096 | 32551 | FBgn0010315 | CycD | 595 | 1582 | CCND1 | 6 | 5.758 | Compara, |
| | | | | | | | | | Inparanoid, |
| | | | | | | | | | Isobase, |
| | | | | | | | | | Phylome, |
| | | | | | | | | | RoundUp, |
| | | | | | | | | | TreeFam |
| CG9096 | 32551 | FBgn0010315 | CycD | 896 | 1585 | CCND3 | 5 | 4.705 | Compara, |
| | | | | | | | | | Inparanoid, |
| | | | | | | | | | orthoMCL, |
| | | | | | | | | | Phylome, |
| | | | | | | | | | TreeFam |
| CG9096 | 32551 | FBgn0010315 | CycD | 891 | 1579 | CCNB1 | 1 | 1.01 | OMA |
| CG9151 | 47080 | FBgn0000028 | acj6 | 5458 | 9219 | POU4F2 | 8 | 7.719 | Compara, |
| | | | | | | | | | Inparanoid, |
| | | | | | | | | | OMA, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG9151 | 47080 | FBgn0000028 | acj6 | 5459 | 9220 | POU4F3 | 7 | 6.709 | Compara, |
| | | | | | | | | | Inparanoid, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG9151 | 47080 | FBgn0000028 | acj6 | 5457 | 9218 | POU4F1 | 7 | 6.654 | Compara, |
| | | | | | | | | | Isobase, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG9156 | 48531 | FBgn0003132 | Pp1-13C | 5501 | 9283 | PPP1CC | 7 | 6.711 | Compara, |
| | | | | | | | | | Homologene, |
| | | | | | | | | | OMA, |
| | | | | | | | | | OrthoDB, |
| | | | | | | | | | orthoMCL, |
| | | | | | | | | | Phylome, |
| | | | | | | | | | TreeFam |
| CG9156 | 48531 | FBgn0003132 | Pp1-13C | 5499 | 9281 | PPP1CA | 6 | 5.711 | Compara, |
| | | | | | | | | | OMA, |
| | | | | | | | | | OrthoDB, |
| | | | | | | | | | orthoMCL, |
| | | | | | | | | | Phylome, |
| | | | | | | | | | TreeFam |
| CG9156 | 48531 | FBgn0003132 | Pp1-13C | 5500 | 9282 | PPP1CB | 1 | 0.9 | orthoMCL |
| CG9324 | 35342 | FBgn0032884 | Pomp | 51371 | 20330 | POMP | 9 | 8.669 | Compara, |
| | | | | | | | | | Inparanoid, |
| | | | | | | | | | Isobase, |
| | | | | | | | | | OMA, |
| | | | | | | | | | OrthoDB, |
| | | | | | | | | | orthoMCL, |
| | | | | | | | | | Phylome, |
| | | | | | | | | | RoundUp, |
| | | | | | | | | | TreeFam |
| CG9326 | 35343 | FBgn0250785 | vari | 51678 | 18167 | MPP6 | 8 | 7.661 | Compara, |
| | | | | | | | | | Homologene, |
| | | | | | | | | | Isobase, |
| | | | | | | | | | OMA, |
| | | | | | | | | | OrthoDB, |
| | | | | | | | | | orthoMCL, |
| | | | | | | | | | Phylome, |
| | | | | | | | | | TreeFam |
| CG9326 | 35343 | FBgn0250785 | vari | 4355 | 7220 | MPP2 | 5 | 4.811 | Compara, |
| | | | | | | | | | OMA, |
| | | | | | | | | | OrthoDB, |
| | | | | | | | | | Phylome, |
| | | | | | | | | | TreeFam |
| CG9326 | 35343 | FBgn0250785 | vari | 8573 | 1497 | CASK | 1 | 1.001 | OrthoDB |
| CG9326 | 35343 | FBgn0250785 | vari | 143098 | 26542 | MPP7 | 1 | 1.001 | OrthoDB |
| CG9326 | 35343 | FBgn0250785 | vari | 4354 | 7219 | MPP1 | 1 | 1.001 | OrthoDB |
| CG9326 | 35343 | FBgn0250785 | vari | 64398 | 18669 | MPP5 | 1 | 1.001 | OrthoDB |
| CG9326 | 35343 | FBgn0250785 | vari | 4356 | 7221 | MPP3 | 1 | 1.001 | OrthoDB |
| CG9326 | 35343 | FBgn0250785 | vari | 58538 | 13680 | MPP4 | 1 | 1.001 | OrthoDB |

### REFERENCES

1. P. B. Crino, K. L. Nathanson, E. P. Henske, The tuberous sclerosis complex. N Engl J Med 355, 1345 (Sep. 28, 2006).
2. J. J. Bissler et al., Sirolimus for angiomyolipoma in tuberous sclerosis complex or lymphangioleiomyomatosis. N Engl J Med 358, 140 (Jan. 10, 2008).
3. F. X. McCormack et al., Efficacy and safety of sirolimus in lymphangioleiomyomatosis. N Engl J Med 364, 1595 (Apr. 28, 2011).
4. D. A. Krueger et al., Everolimus for subependymal giant-cell astrocytomas in tuberous sclerosis. N Engl J Med 363, 1801 (Nov. 4, 2010).
5. W. G. Kaelin, Jr., Molecular biology. Use and abuse of RNAi to study mammalian gene function. Science 337, 421 (Jul. 27, 2012).
6. S. E. Mohr, J. A. Smith, C. E. Shamu, R. A. Neumuller, N. Perrimon, RNAi screening comes of age: improved techniques and complementary approaches. Nat Rev Mol Cell Biol 15, 591 (September, 2014).
7. A. H. Tong et al., Systematic genetic analysis with ordered arrays of yeast deletion mutants. Science 294, 2364 (Dec. 14, 2001).
8. A. H. Tong, C. Boone, Synthetic genetic array analysis in Saccharomyces cerevisiae. Methods Mol Biol 313, 171 (2006).
9. T. Horn et al., Mapping of signaling networks through synthetic genetic interaction analysis by RNAi. Nat Methods 8, 341 (April, 2011).
10. O. Nir, C. Bakal, N. Perrimon, B. Berger, Inference of RhoGAP/GTPase regulation using single-cell morphological data from a combinatorial RNAi screen. Genome research 20, 372 (March, 2010).
11. A. R. Bassett, C. Tibbit, C. P. Ponting, J. L. Liu, Mutagenesis and homologous recombination in Drosophila cell lines using CRISPR/Cas9. Biol Open 3, 42 (2014).
12. R. Bottcher et al., Efficient chromosomal gene modification with CRISPR/cas9 and PCR-based homologous recombination donors in cultured Drosophila cells. Nucleic Acids Res 42, e89 (2014).
13. L. Cong et al., Multiplex genome engineering using CRISPR/Cas systems.

Science 339, 819 (Feb. 15, 2013).
14. S. J. Gratz et al., Genome engineering of Drosophila with the CRISPR RNA-guided Cas9 nuclease. Genetics 194, 1029 (August, 2013).
15. P. Mali et al., RNA-guided human genome engineering via Cas9. Science 339, 823 (Feb. 15, 2013).
16. X. Ren et al., Optimized gene editing technology for Drosophila melanogaster using germ line-specific Cas9. Proc Natl Acad Sci USA 110, 19012 (Nov. 19, 2013).
17. A. R. Bassett, C. Tibbit, C. P. Ponting, J. L. Liu, Highly efficient targeted mutagenesis of Drosophila with the CRISPR/Cas9 system. Cell Rep 4, 220 (Jul. 11, 2013).
18. Z. L. Sebo, H. B. Lee, Y. Peng, Y. Guo, A simplified and efficient germline-specific CRISPR/Cas9 system for Drosophila genomic engineering. Fly (Austin) 8, 52 (January-March, 2014).
19. Z. Yu et al., Highly efficient genome modifications mediated by CRISPR/Cas9 in Drosophila. Genetics 195, 289 (September, 2013).
20. Y. Fu et al., High-frequency off-target mutagenesis induced by CRISPR-Cas nucleases in human cells. Nat Biotechnol 31, 822 (September, 2013).
21. P. D. Hsu et al., DNA targeting specificity of RNA-guided Cas9 nucleases. Nat Biotechnol 31, 827 (September, 2013).
22. P. Mali et al., CAS9 transcriptional activators for target specificity screening and paired nickases for cooperative genome engineering. Nat Biotechnol 31, 833 (September, 2013).
23. V. Pattanayak et al., High-throughput profiling of off-target DNA cleavage reveals RNA-programmed Cas9 nuclease specificity. Nat Biotechnol 31, 839 (September, 2013).
24. M. Jinek et al., A programmable dual-RNA-guided DNA endonuclease in adaptive bacterial immunity. Science 337, 816 (Aug. 17, 2012).
25. J. G. Doench et al., Rational design of highly active sgRNAs for CRISPR-Cas9-mediated gene inactivation. Nat Biotechnol, (Sep. 3, 2014).
26. T. Wang, J. J. Wei, D. M. Sabatini, E. S. Lander, Genetic screens in human cells using the CRISPR-Cas9 system. Science 343, 80 (Jan. 3, 2014).
27. O. Shalem et al., Genome-scale CRISPR-Cas9 knockout screening in human cells. Science 343, 84 (Jan. 3, 2014).
28. W. Fujii, K. Kawasaki, K. Sugiura, K. Naito, Efficient generation of large-scale genome-modified mice using gRNA and CAS9 endonuclease. Nucleic Acids Res 41, e187 (Nov. 1, 2013).
29. L. E. Jao, S. R. Wente, W. Chen, Efficient multiplex biallelic zebrafish genome editing using a CRISPR nuclease system. Proc Natl Acad Sci USA 110, 13904 (Aug. 20, 2013).
30. R. A. Lindquist et al., Genome-scale RNAi on living-cell microarrays identifies novel regulators of Drosophila melanogaster TORC1-S6K pathway signaling. Genome research 21, 433 (March, 2011).
31. R. A. Neumuller et al., Stringent analysis of gene function and protein-protein interactions using fluorescently tagged genes. Genetics 190, 931 (March, 2012).
32. L. Cherbas, R. Moss, P. Cherbas, Transformation techniques for Drosophila cell lines. Methods Cell Biol 44, 161 (1994).
33. H. Lee et al., DNA copy number evolution in Drosophila cell lines. Genome Biol 15, R70 (Aug. 28, 2014).
34. T. Nobukini, G. Thomas, The mTOR/S6K signalling pathway: the role of the TSC1/2 tumour suppressor complex and the proto-oncogene Rheb. Novartis Found Symp 262, 148 (2004).
35. J. Huang, B. D. Manning, The TSC1-TSC2 complex: a molecular switchboard controlling cell growth. Biochem J 412, 179 (Jun. 1, 2008).
36. D. A. Guertin, K. V. Guntur, G. W. Bell, C. C. Thoreen, D. M. Sabatini, Functional genomics identifies TOR-regulated genes that control growth and division. Curr Biol 16, 958 (May 23, 2006).
37. D. J. Kwiatkowski, B. D. Manning, Tuberous sclerosis: a GAP at the crossroads of multiple signaling pathways. Hum Mol Genet 14 Spec No. 2, R251 (Oct. 15, 2005).
38. N. Tapon, N. Ito, B. J. Dickson, J. E. Treisman, I. K. Hariharan, The Drosophila tuberous sclerosis complex gene homologs restrict cell growth and cell proliferation. Cell 105, 345 (May 4, 2001).
39. C. G. Proud, Regulation of protein synthesis by insulin. Biochem Soc Trans 34, 213 (April, 2006).
40. M. K. Holz, B. A. Ballif, S. P. Gygi, J. Blenis, mTOR and S6K1 mediate assembly of the translation preinitiation complex through dynamic protein interchange and ordered phosphorylation events. Cell 123, 569 (Nov. 18, 2005).
41. K. G. Guruharsha et al., A protein complex network of Drosophila melanogaster. Cell 147, 690 (Oct. 28, 2011).
42. S. Lee et al., The F-actin-microtubule crosslinker Shot is a platform for Krasavietz-mediated translational regulation of midline axon repulsion. Development 134, 1767 (May, 2007).
43. A. A. Kiger et al., A functional genomic analysis of cell morphology using RNA interference. Journal of biology 2, 27 (2003).
44. J. T. Lis, P. Mason, J. Peng, D. H. Price, J. Werner, P-TEFb kinase recruitment and function at heat shock loci. Genes & development 14, 792 (Apr. 1, 2000).
45. S. Wilkinson et al., The cyclin-dependent kinase PITSLRE/CDK11 is required for successful autophagy. Autophagy 7, 1295 (November, 2011).
46. A. A. Parkhitko et al., Autophagy-dependent metabolic reprogramming sensitizes TSC2-deficient cells to the antimetabolite 6-aminonicotinamide. Mol Cancer Res 12, 48 (January, 2014).
47. P. S. Lee et al., Rapamycin-insensitive up-regulation of MMP2 and other genes in tuberous sclerosis complex 2-deficient lymphangioleiomyomatosis-like cells. American journal of respiratory cell and molecular biology 42, 227 (February, 2010).
48. D. Medvetz, C. Priolo, E. P. Henske, Therapeutic targeting of cellular metabolism in cells with hyperactive mTORC1: A paradigm shift. Mol Cancer Res, (Oct. 8, 2014).
49. E. A. Bach et al., GFP reporters detect the activation of the Drosophila JAK/STAT pathway in vivo. Gene expression patterns: GEP 7, 323 (January, 2007).
50. R. Sopko et al., Combining genetic perturbations and proteomics to examine kinase-phosphatase networks in Drosophila embryos. Developmental cell 31, 114 (Oct. 13, 2014).
51. H. Zhang et al., Loss of Tsc1/Tsc2 activates mTOR and disrupts PI3K-Akt signaling through downregulation of PDGFR. The Journal of clinical investigation 112, 1223 (October, 2003).
52. C. Li et al., Estradiol and mTORC2 cooperate to enhance prostaglandin biosynthesis and tumorigenesis in TSC2-deficient LAM cells. The Journal of experimental medicine 211, 15 (Jan. 13, 2014).

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

1. A method for treating a subject having a cell proliferative disorder characterized by proliferating cells that: (i) overexpress mTOR or (ii) have increased mTOR complex 1 (mTORC1) activity, the method comprising administering to the subject a compound that inhibits IMPDH1, IMPDH2, RNGTT, RNMT, Cdk11, Cdk9, CCNT1, CCND3, Cyclin L1, or Cyclin L2 in an amount effective to treat the cell proliferative disorder.

2. The method according to claim 1, wherein the proliferating cells comprise at least one mutation in one or both of the TSC1 and TSC2 genes.

3. The method according to claim 1, wherein the cell proliferative disorder is a cancer.

4. The method according to claim 3, wherein the cancer is a lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer.

5. The method according to claim 1, wherein the cell proliferative disorder is tuberous sclerosis complex, lymphangioleiomyomatosis, a PTEN mutant hamartoma syndrome, Peutz Jeghers syndrome, Familial Adenomatous Polyposis, or neurofibromatosis type 1.

6. The method according to claim 5, wherein the PTEN mutant hamartoma syndrome is Cowden disease, Proteus disease, Lhermitte-Duclos disease, or Bannayan-Riley-Ruvalcaba syndrome.

7. The method according to claim 1, wherein the compound binds to and inhibits the activity of IMPDH1, IMPDH2, RNGTT, RNMT, Cdk11, Cdk9, CCNT1, CCND3, Cyclin L1, or Cyclin L2.

8. The method according to claim 7, wherein the compound is a small molecule, a macrocycle compound, a polypeptide, a nucleic acid, or a nucleic acid analog.

9. The method according to claim 1, wherein the compound reduces the expression or stability of an mRNA encoding IMPDH1, IMPDH2, RNGTT, RNMT, Cdk11, Cdk9, CCNT1, CCND3, Cyclin L1, or Cyclin L2 protein.

10. The method according to claim 9, wherein the compound is an antisense oligonucleotide, an siRNA, an shRNA, or a ribozyme.

11. The method according to claim 1, comprising determining whether the proliferating cells overexpress mTOR or have increased mTOR complex 1 (mTORC1) activity.

12. The method according to claim 1, comprising, prior to administering the compound to the subject, requesting the results of a test that determined whether the proliferating cells overexpress mTOR or have increased mTOR complex 1 (mTORC1) activity.

13. The method according to claim 2, comprising determining whether the proliferating cells comprise at least one mutation in one or both of the TSC1 and TSC2 genes.

14. The method according to claim 2, comprising, prior to administering the compound to the subject, requesting the results of a test that determined whether the proliferating cells comprise at least one mutation in one or both of the TSC1 and TSC2 genes.

15. A method for treating a subject having a cell proliferative disorder, the method comprising administering to the subject a compound that inhibits IMPDH1, IMPDH2, RNGTT, RNMT, Cdk11, Cdk9, CCNT1, CCND3, Cyclin L1, or Cyclin L2 in an amount effective to treat the cell proliferative disorder, wherein the subject has been identified as having a cell proliferative disorder characterized by proliferating cells that: (i) overexpress mTOR or (ii) have increased mTOR complex 1 (mTORC1) activity.

16. A method for treating a subject having a proliferative disorder characterized in that one or both of the TSC1 and TSC2 genes are mutated, the method comprising administering to the subject a compound that inhibits IMPDH1, IMPDH2, RNGTT, RNMT, Cdk11, Cdk9, CCNT1, CCND3, Cyclin L1, or Cyclin L2, in an amount effective to treat the cell proliferative disorder.

17. The method according to claim 15, wherein the compound binds to and inhibits the activity of IMPDH1, IMPDH2, RNGTT, RNMT, Cdk11, Cdk9, CCNT1, CCND3, Cyclin L1, or Cyclin L2.

18. The method according to claim 17, wherein the compound is a small molecule, a macrocycle compound, a polypeptide, a nucleic acid, or a nucleic acid analog.

19. The method according to claim 15, wherein the compound reduces the expression or stability of an mRNA encoding IMPDH1, IMPDH2, RNGTT, RNMT, Cdk11, Cdk9, CCNT1, CCND3, Cyclin L1, or Cyclin L2 protein.

20. The method according to claim 19, wherein the compound is an antisense oligonucleotide, an siRNA, an shRNA, or a ribozyme.

21-45. (canceled)
